(19) United States
(12) Patent Application Publication
(10) Pub. No.: US 2023/0417770 A1
(43) Pub. Date: Dec. 28, 2023

(54) MARKERS AND METHODS FOR DIAGNOSING HEATSTROKE

(71) Applicants: National Guard Health Affairs, Riyadh (SA); King Saud bin Abdulaziz University for Health Sciences, Riyadh (SA); King Abdullah International Medical Research Center, Riyadh (SA)

(72) Inventor: Abderrezak BOUCHAMA, Riyadh (SA)

(73) Assignees: National Guard Health Affairs, Riyadh (SA); King Saud bin Abdulaziz University for Health Sciences, Riyadh (SA); King Abdullah International Medical Research Center, Riyadh (SA)

(21) Appl. No.: 18/326,700

(22) Filed: May 31, 2023

Related U.S. Application Data
(60) Provisional application No. 63/348,193, filed on Jun. 2, 2022.

(51) Int. Cl. G01N 33/68

(52) U.S. Cl. CPC G01N 33/6893; G01N 2800/40

(57) ABSTRACT

Methods for medical diagnosis of heat-related health conditions including heatstroke, compositions comprising RNA or protein biomarkers for heatstroke and heat-associated conditions, probes and primers and antibodies to such biomarkers.

### CROSS-REFERENCE TO A RELATED APPLICATION

This application claims priority to U.S. Provisional 63/348,193, filed Jun. 2, 2022, which is incorporated by reference for all purposes.

### REFERENCE TO A SEQUENCE LISTING

In accordance with 37 CFR § 1.833-1835 and 37 CFR § 1.77(b)(5), the specification makes reference to a Sequence Listing submitted electronically as a .xml file named “543455US_ST26.xml”. The .xml file was generated on May 31, 2023 and is 42,940 bytes in size. The entire contents of the Sequence Listing are hereby incorporated by reference.

### STATEMENT REGARDING PRIOR DISCLOSURES BY AN INVENTOR OR

### JOINT INVENTOR

Related technology is described by Schlader, et al., Biomarkers of heatstroke-induced organ injury and repair, Jun. 2, 2022, EXPERIMENTAL PHYSIOLOGY, <hypertext transfer protocol secure://doi.org/10.1113/EP090142> which is incorporated by reference for all purposes.

### BACKGROUND OF THE INVENTION

### Field of the Invention

The invention pertains to medical diagnosis of heatstroke.

### Related Art

Environmental heat has recently emerged as the most consistent threat of climate change. Waits, N, et al. The 2020 report of The Lancet Countdown on health and climate change: responding to converging crises. LANCET, 397(10269):129-170 (2021); Vicedo-Cabrera, A M, et al. The burden of heat-related mortality attributable to recent human-induced climate change. NAT CLIM CHANG, 11(6):492-500 (2021); and Mora, C, et al. Global risk of deadly heat. NATURE CLIMATE CHANGE, 7:501 (2017). Heatwaves have become more frequent, with higher intensity, and lasting longer across the planet resulting in an increasing number of heat-related morbidity and mortality, particularly from heatstroke. Mora, C, et al.; Semenza, J C, et al. Heat-related deaths during the July 1995 heat wave in Chicago. THE NEW ENGLAND JOURNAL OF MEDICINE, 335(2):84-90 (1996); Fouillet, A, et al. Excess mortality related to the August 2003 heat wave in France, INT ARCH OCCUP ENVIRON HEALTH (2006); and Barriopedro, D, The hot summer of 2010: redrawing the temperature record map of Europe. SCIENCE (New York, NY), 332(6026):220-224 (2011). Classic heatstroke is a life-threatening condition characterized by uncontrolled hyperthermia exceeding 40.1° C. and multiple organ dysfunction secondary to passive exposure to extreme environmental heat. Bouchama, A, et al. Classic and exertional heatstroke. NATURE REVIEWS DISEASE PRIMERS 8(1):8 (2022). Heatstroke can also occur following strenuous exercise, even in temperate weather, and affect mainly highly trained athletes and military and construction workers. In contrast, classic heatstroke manifests in epidemic form, predominantly in the elderly population with associated comorbidities. Several hundreds of people died from heatstroke during the Chicago in the USA, France, and Russian recent heat waves. Semenza, J C, et al.; Fouillet, A., et al; and Barriopedro, D., et al. Regardless of the etiology, heatstroke is a medical emergency, invariably fatal from multiple organ damage in a few hours if left untreated. Besides physical cooling, there is no specific therapy available because the pathogenesis of he

Heatstroke in humans at a molecular level is not fully understood, and thus no therapeutic targets have been identified so far. Exposure to extreme heat is significant stress for most living species, including humans, as it can induce macromolecular damage, including proteins, lipid membranes, and DNA. Lopez-Maury, L, et al. Tuning gene expression to changing environments: from rapid responses to evolution adaptation. NATURE REVIEWS GENETICS 2008, 9(8):583-593; Kuliz, D, Molecular and evolutionary basis of the cellular stress response. ANNUAL REVIEW OF PHYSIOLOGY 2005, 67:225-257; Richter, K, et al. The heat shock response: life on the verge of death. MOLECULAR CELL, 40(2):253-266 (2010); Mahat D B, et al. Mammalian Heat Shock Response and Mechanisms Underlying Its Genome-wide Transcriptional Regulation. MOLECULAR CELL, 62(1):63-78 (2016); and Bouchama, A, et al A Model of Exposure to Extreme Environmental Heat Uncovers the Human Transcriptome to Heat Stress, SCIENTIFIC REPORTS, 7(1):9429 (2017). Accordingly, all living species have a heat stress response (HSR) strategy to shield against heat-induced macromolecular damage and the intracellular expression of heat shock proteins (HSPs) is a universal and conserved response across all kingdoms of living organisms. Prior studies have measured increased concentrations of heat shock proteins in mammalian tissues in response to heat stress.

The HSR involves rapid reprograming of the transcriptome to stress-related function and redirection of energy towards this end at the expense of growth and proliferation. However, the molecular mechanisms regulating the HSR in humans exposed to extreme environmental heat and how these mechanisms fail to prevent the progression to heatstroke have yet to be elucidated.

The inventors have previously shown that young and healthy humans exposed to extreme heat in a controlled environment such as a sauna initiate an HSR reminiscent of yeast, fly, and worm responses, suggesting a high degree of evolutionary conservation. Bouchama, A., et al., supra. An earlier study on four healthy young soldiers showed that exertional heat injury occurs despite a potent HSR. Sonna, L A, et al. Exertional heat injury and gene expression changes: a DNA microarray analysis study. J APPL PHYSIOL, 96(5):1943-1953. (2004). Recently, a study combining transcriptomic and proteomics in a preclinical rat model of classic heatstroke suggested that a failure of the mechanisms that preserve the proteome and produce sufficient bioenergy to sustain metabolism during severe heat stress may underlie the progression toward heatstroke. Stallings, J D, et al. Patterns of gene expression associated with recovery and injury in heat-stressed rats. BMC GENomics, 15(1):1058 (2014). The molecular mechanisms by which individuals exposed to extreme environmental heat progress to life-threatening heatstroke has not been well understood and thus there is a need for methods using biomarkers that can help differentially identify subjects having different heat-associated conditions.

In view of the lack of a suitable set of biomarkers for heatstroke, the inventors proposed to perform comparative transcriptomic measurements of the HSR in a cohort of subjects exposed to the same environmental conditions with and without heatstroke to identify molecular mechanisms underlying the progression or not to heatstroke and to identify the genomic signature of heatstroke.

As disclosed herein, the inventors used a whole genome microarray to characterize the genomic profile of heatstroke in a unique cohort of patients with severe classic heatstroke during the Muslim pilgrimage in the desert climate of Mecca. The inventors also examined 330 members of the human chaperome family which comprises an ensemble of all cellular molecular chaperones (HSPs) and co-chaperone proteins.

Based on this work, the inventors unraveled the molecular signature of heatstroke including the identification of nine specific HSPs which can serve to diagnose classic and exertional heatstroke and related conditions.

### SUMMARY

This section provides a brief, non-limited overview of some aspects of the technology disclosed herein.

Detection and diagnosis of heatstroke and related conditions. One aspect of this technology is directed to a method for diagnosing heatstroke, including nonexertional or classic heatstroke, as well as exertional heatstroke or other heat-related conditions, comprising detecting altered expression of one, two, three, four, five, six, seven, eight, nine or more RNA biomarkers in a biological sample from a subject compared to a control subject not having heatstroke; and, when altered expression is detected, treating the subject for heatstroke or a heat-related condition; wherein said biomarkers encode at least one heat shock protein from the HSP70 family, from the HSPB family, or from the HSP40 family, and/or FKBP prolyl isomerase 4. Advantageously, the one, two, three, four, five, six, seven, eight ; nine or more biomarkers may be selected from those encoding HSPA1A, HSPA1B, HSPA6, HSPA4L, HSPB1, DNAJA4, DNAJB1, DNAJB4, and/or FKBP4 (FKBP Prolyl Isomerase 4).

This method may detect at least one, two, three, four, five, six, seven, eight, nine, or more RNA biomarkers isolated from a blood product such as whole blood, buffy coat, peripheral blood mononuclear cells (PBMCs) or other cellular fractions of blood, or other biological samples, including RNA containing blood, plasma, serum, cerebrospinal fluid, bronchial lavage fluid, saliva, urine, or other solid or liquid biological samples containing RNA or the heat-associated proteins described herein. Preferably, the biomarkers are isolated from a whole blood, buffy coat or PBMCs of a subject being evaluated for heatstroke or another heat-related condition.

Heat shock Proteins mRNA is typically expressed inside cells and expresses intracellular chaperones that can bind to nascent proteins or to mature proteins that are denatured (e.g. misfolded or aggregated) by stress such as by extreme heat. The HSPs function as chaperone and assist or repair the proteins to regain their 3D configuration and become functional. If they fail to repair the denatured proteins, they could also direct them to degradation pathways (e.g., ubiquitin-proteasome or autophagy pathways) for elimination. Some HSPs have been found in the circulation and may have been released by cell death or necrosis. These could potentially serve as prognostic markers.

In an alternative embodiment, a method may detect one or more heat-associated proteins described by HSPA1A, HSPA1B, HSPA6, HSPA4L, HSPB1, DNAJA4, DNAJB1, DNAJB4, and/or FKBP4 (FKBP Prolyl Isomerase 4) for example, using ELISA and antibodies recognizing these proteins or other biochemical and immunological methods.

In this method the biomarkers may be isolated from a subject who has a rectal, oral, axillary, tympanic and/or temporal artery body temperature of ≥36, 37, 38, 39, 40, 41, 42 or 43° C., preferably having an elevated body temperature compared to a normal value for the subject.

In some embodiments, the biomarkers are isolated from a subject experiencing confusion, agitation, irritability, delirium, seizures or coma and who has a rectal, oral, axillary, tympanic or temporal artery body temperature of ≥36, 37, 38, 39, 40, 41, 42 or 43° C., preferably a temperature of 37° C. or more; in other embodiments, the biomarkers are isolated from a subject has cool, moist skin (or more often hot and dry skin) when in the heat, with and/or without profuse sweating, faintness, dizziness, fatigue, weak and rapid pulse, low blood pressure upon standing, muscle cramps, nausea, and/or headache and who has a rectal, oral, axillary, tympanic or temporal artery body temperature of ≥32, 33, 34, 35, 36, 37, 38, 39, 40, 41, 42 or 43° C. A subject having one or more symptoms of heat stroke may optionally be treated after diagnosis of heat stroke or a related condition.

In some embodiments, the biomarkers are isolated from a subject exposed to an environment having a heat index of ≥32, 33, 34, 35, 36, 37, 38, 39 or 40° C.

In some embodiments, the biomarkers are isolated a subject, who is an amateur or professional athlete, or a laborer, exposed to an environment having a heat index of ≥40, 41, 42, 43, 44, 45, 46, 47, 48, 49, 50, 51, 52, 53, to 54° C.

In other embodiments, the biomarkers are isolated from a subject, who is an amateur or professional athlete, or a laborer, exposed to an environment having a heat index of ≥40, 41, 42, 43, 44, 45, 46, 47, 48, 49, 50, 51, 52, 53, to 54° C. Subjects, such as laborers or athletes may be from outside the geographic area of work or sport and unaccustomed to local heat conditions.

In some embodiments of the methods disclosed herein at least one of the biomarkers encodes a protein comprising HSP A1A, HSP A1B, HSP A6, or HSP A4L.

The biomarkers as disclosed herein include full-length coding sequences for a corresponding heat shock protein as well as identifiable fragments thereof, for example, partially digested or degraded RNA, such as a partial sequence of a biomarker to which can bind to a probe or primer. In some embodiments, the methods disclosed herein exclude HSP A1 and other markers disclosed by Bouchama, et al., A model of exposure to extreme environmental heat uncovers the human transcriptome to heat stress. SCIENTIFIC REPORTS, 2017, 7, 9429. Besides HSP A1, which is moderately expressed, none of the eight other biomarkers disclosed herein were shown to be expressed in that study.

In some embodiments disclosed herein at least one of the biomarkers encodes a heat shock protein comprising HSP B1. HSP B1 is distinct from other HSPs like HSP B8 and HSP B6, even though it belongs to the same family of small heat shock proteins. Similarly, HSP B11 and HSP 90AB1 need not be included in a basic panel of RNA biomarkers of the invention.

In some embodiments of the methods disclosed herein at least one of the biomarkers encodes a heat shock protein comprising DNAJA4, DNAJB1, or DNAJB4.

In some embodiments of the methods disclosed herein at least one of the biomarkers encodes FKBP4 (FKBP Prolyl Isomerase 4).

In some embodiments of the methods disclosed herein the biomarkers encode at least one of HSPA1A, HSPA1B, HSPA6, or HSPA4L; HSPB1; at least one of DNAJA4, DNAJB1, or DNAJB4; and/or FKBP4 (FKBP Prolyl Isomerase 4).

In some embodiments of the methods disclosed herein the biomarkers encode HSPA1A, HSPA1B, HSPA6, HSPA4L, HSPB1, DNAJA4, DNAJB1, DNAJB4, and FKBP4 (FKBP Prolyl Isomerase 4).

In other embodiments, the methods disclosed herein may he performed with a subset of these biomarkers, such as with HSPA1A, HSPA4L, HSPB1, and FKBP4, or with three biomarkers such as HSPA1B, HSPA4L, and HSPB1. Other combinations of these biomarkers may be used including HSPA1B, HSPA4L, DNAJA4, HSPB1 and FKBP4.

Treatments of heatstroke and associated conditions. Upon diagnosis or detection of elevations in the biomarkers disclosed herein various modes of treatment of heatstroke or other heat-related conditions can be administered.

The disclosed methods may further comprise treating a subject for heatstroke or a heat-related condition or the prophylactic administration of such treatments for a subject who has not yet developed heatstroke but is at risk of heatstroke due to physical, medical, pharmaceutical, or environmental conditions.

A subject's body temperature may be lowered, especially core temperature, by contacting all or part of the subject's body with icepacks, ice water, outer coolants, administering cool or hydrating fluids such as water or electrolyte solutions (e.g., containing salts of sodium, potassium, calcium, magnesium, phosphorous, chlorides, etc.). A subject may also he moved to a shady area that is not directly irradiated by sunlight. Placement of a subject in a shady area can reduce the heat index value by at least 1, 2, 3, 4, 5, 6, 7 or 8° C.

Treatment may comprise external physical cooling such as conduction-, convection-, or evaporative-based cooling of the subject. These include, but are not limited to immersion in iced water, placement of ice packs or cool wet towels on the neck, axillae and groin; or spraying the skin with cool to tepid water (20-30° C.) combined with continuous fanning.

Probes and Primers recognizing biomarkers. Another aspect of this technology involves a composition comprising probes or primers that bind to or amplify at least two polynucleotide biomarkers encoding HSPA1A, HSPA1B, HSPA6, HSPA4; HSPB1; DNAJA4, DNAJB1, DNAJB4; and/or FKBP4 (FKBP Prolyl Isomerase 4). Such a composition may comprise probes or primers that detect at least 2, 3, 4, 5, 6, 7, 8 or 9 of said biomarkers. Such a composition may be part of a microarray or RNA sequencing. Microarrays suitable for measuring polynucleotide biomarkers described herein are described by, and incorporated by reference to, METHODS MOL BIOL. 2011; 671:3-34. doi: 10.1007/978-1-59745-551-0_1.

Another feature of this technology is directed to a kit comprising at least one probe or primer that can bind to or amplify a biomarker that is HSPA1A, HSPA1B, HSPA6, HSPA4L, (HSP70 family); HSPB1 (heat shock family B): DNAJA4, DNAJB1, DNAJB4 (HSP40 family); or FKBP4 (FKBP Prolyl Isomerase 4) and optionally containers for said probes or primers, reagents for detection of said biomarkers, instructions for use, or other components, supplies or equipment for detecting said biomarkers.

The foregoing paragraphs have been provided by way of general introduction and are not intended to limit the scope of the claims. The described embodiments, together with further advantages, will be best understood by reference to the following detailed description taken in conjunction with the accompanying drawings.

### BRIEF DESCRIPTION OF THE DRAWINGS

This application file contains at least one drawing executed in color. Copies of this patent application publication with color drawing(s) will be provided by the Office upon request and payment of the necessary fee.

FIG. 1. The core temperatures of 19 heatstroke patients upon admission and presentation to the hospital before cooling (T0).

FIGS. 2A-2F. Transcriptional signatures of heatstroke. See below for detail.

FIG. 2A. Bar chart depicting the number of genes that are differentially upregulated (red, first bar) or down-regulated (blue, second bar) immediately pre-(T0) and 4±3 hours (T1) post-cooling relative to heat-stressed controls.

FIG. 2B. Venn diagrams showing the number f differentially expressed (DE) genes at T0 and at T1 after cooling. The number 4911 in the overlapping circle represents the number of DE genes shared by both T0 and T1.

FIG. 2C. Heat map representation of all genes as ordered by hierarchical clustering. Upregulated genes are shown in red and down-regulated genes in blue. DE genes shown for Control, T0 and T1.

FIG. 2D. Principal component analysis (PCA) of the transcriptional data.

FIGS. 2E and 2F. Volcano plots of differentially expressed genes in heatstroke patients FIG. 2E (T0) and FIG. 2F (T1) relative to heat-stressed controls. In FIG. 2E, genes with significant differences in both expression (log] 0 P<0.05) and log2 (fold change>1.3) are shown in red. Blue indicates genes significant by P-value but not reaching the fold-changes threshold, and green, genes reaching the fold changes threshold but not the p-value<0.05. Red circle indicates selected biomarkers for diagnosing heatstroke. Genes with significant differences in both expression (log10 P<0.05) and log2 (fold change>1.3) are shown in red. Blue indicates genes significant by P-value but not reaching the fold changes threshold, and green genes reaching the fold changes threshold but not the p-value<0.05.

FIGS. 3A to 3D. Heat shock response in patients with heatstroke before (T0) and after cooling (T1). See below for detail.

FIG. 3A. Heat map showing expression of chaperone-related. genes in patients with heatstroke precooling (T0), and post-cooling (T1) relative to heat-stressed controls. Upregulated genes are shown in red and down-regulated genes in blue. Chaperone genes are displayed in green, and cochaperones in purple color. ATP dependent and ATP independent and category of HSPs are indicated by various colors displayed in the enclosed figure legend.

FIG. 3B-1 to 3B-2, Functional HSP family number and their location within the cell are indicated (see categories on right).

FIG. 3B-1: chaperones differentially expressed at T0.

FIG. 3B-2 chaperones differentially expressed at T1.

FIG. 3C. Box plots depicting selected heat-inducible HSP genes that are differentially upregulated or down-regulated in heatstroke patients on admission (T0) and 4 hours after cooling therapy (T1) relative to heat-stressed control subjects. This figure describes HSPA1A, HSPA1B, HSPA6, HSPA4L, HSPB1, DNAJA4, DNAJB1, DNAJB4 and/or FKBP4 (FKBP Prolyl Isomerase 4).

FIG. 3D. Canonical pathways associated with proteostasis at T0 and. T1.

The pathways are ranked by the Z-score calculated by IPA using Fisher's exact test, right-tailed. A Z-score≥1 means that a function is significantly increased (orange), whereas a Z-score≤−1 indicates a significantly decreased function (blue), and an undetermined prediction in gray. The pathway analyses were generated through the use of QIAGEN's Ingenuity Pathway Analysis (IPA®, QIAGEN Redwood City, worldwideweb.qiagen.com/ingenuity).

FIGS. 4A and 4B. HSF1 and HSF2 upstream regulators in heatstroke patients at presentation. Ninety-one and seventeen genes are predicted as direct HSF1 and HSF2 downstream targets in heatstroke patients prior to cooling (T0). Arrows indicate the predicted relationship: orange=leads to activation, blue=leads to inhibition, grey=effect not predicted, and yellow=findings inconsistent with the state of the downstream molecule. The red color indicates genes are upregulated and the green color indicates genes are downregulated in heatstroke patients. The intensity of the color reflects the level of up or downregulation.

FIGS. 5A and 5B. Metabolism associated with energy in heatstroke patients.

FIG. 5A. Canonical pathways associated with the metabolism of energy at T0 and T1. The pathways are ranked by the Z-score calculated by IPA using Fisher's exact test, right-tailed. A Z-score≥1 means that a function is significantly increased (orange), whereas a Z-score≤−1 indicates a significantly decreased function (blue), and an undetermined prediction in gray. The intensity of the color reflects the level of activation or inhibition. The pathway analyses were generated through the use of QIAGEN's Ingenuity Pathway Analysis (IPA®, QIAGEN Redwood City, www.qiagen.com/ingenuity).

FIG. 5B. The pathway for glycolysis and oxidative phosphorylation is shown along with the genes that were significantly expressed after heatstroke on admission (T0) and after cooling (T1). An increase in gene expression (reel), a decrease (green), unchanged (grey), or a gene not present in the data set (white).

FIGS 6A and 6B. DNA damage response in patients with heatstroke pre- and post-cooling. FIG. 6A. Functional DDR genes number in patients with heat stroke pre-cooling (T0).

FIG. 6B. DNA damage response in patients with heatstroke pre- and post-cooling. Activation z-scores for canonical pathways at time T0 and time T1. Canonical pathways associated with DDR at T0 and T1. The pathways are ranked by the Z-score calculated by IPA using Fisher's exact test, right-tailed. A Z-score≥1 means that a function is significantly increased (orange), whereas a Z-score≤−1 indicates a significantly decreased function (blue) and an undetermined prediction in gray. The intensity of the color reflects the level of activation or inhibition. The pathway analyses were generated through the use of QIAGEN's Ingenuity Pathway Analysis (IPA®, QIAGEN Redwood City. www.qiagen.com/ingenuity).

FIGS. 7A-7C. Metabolic and signaling pathways related to immune response, CNS, and cellular growth proliferation and development after heatstroke on admission and post-cooling. The pathways are ranked by the Z-score calculated by IPA using Fisher's exact test, right-tailed. A Z-score≥1 means that a function is significantly increased (orange), whereas a Z-score≤−1 indicates a significantly decreased function (blue), and an undetermined prediction in gray. The intensity of the color reflects the level of activation or inhibition. The pathway analyses were generated through the use of QIAGEN's Ingenuity Pathway Analysis (IPA®, QIAGEN Redwood City.

Immune response z-scores for canonical pathways at T0 and T1 (FIG. 7A).

Central nervous system signaling z-scores for canonical pathways at T0 and T1 (FIG. 7B).

Cellular growth, proliferation and development z-scores for canonical pathways at T0 and T1 (FIG. 7C).

FIG. 8. Diagram of EIF2 signaling pathway with overlaid molecular activity prediction after heatstroke. This depicts canonical EIF2 signaling pathways showing down (green) regulated genes immediately after heatstroke (T0), with translation elongation and stress granules assembly, predicted to be decreased (colored blue). Endoplasmic Reticulum (ER) stress response and apoptosis are predicted to be increased (orange). The detailed legend is shown below. The pathway and the molecular activity prediction analyses were generated through the use of QIAGEN's Ingenuity Pathway Analysis (IPA®, QIAGEN Redwood City, www.qiagen.ingenuity).

FIG. 9. Diagram of UPR signaling pathway with overlaid prediction of molecular activity after heatstroke. Diagram of canonical UPR signaling pathway showing up (red) and down (green) regulated genes immediately after heatstroke (T0), with protein refolding, protein degradation, initiation of protein translation, and apoptosis predicted to be increased (colored orange). Detailed legend displayed with FIG. 8.

FIG. 10. Diagram of Ubiquitin-Proteasome signaling pathway with overlaid prediction of molecular activity after heatstroke. Diagram of protein ubiquitination pathway showing up down (green) regulated genes immediately after heatstroke (T0), with protein refolding, predicted increased (colored orange) and monoubiquitylation predicted decreased (colored blue). All the proteasome endopeptidase genes, including those of the immunoproteasome, are downregulated (green). Detailed legend displayed with FIG. 8.

FIG. 11. Diagram of Mitochondrial Dysfunction signaling pathway with overlaid prediction of molecular activity after heatstroke. Diagram of mitochondrial dysfunction signaling pathway showing up down (green) regulated genes immediately after heatstroke (T0), with mitochondrial fragmentation, apoptosis, and ATP predicted decreased (colored blue), and oxidative stress predicted increased (colored orange). Detailed legend displayed with FIG. 8.

FIG. 12. Diagram of Amyloid processing signaling pathway with overlaid prediction of molecular activity after heatstroke. Diagram of amyloid signaling pathway showing up down (green) regulated genes immediately after heatstroke (T0), with increased senile plaque, microtubule instability, and membrane damage (colored orange). Detailed legend displayed with FIG. 8.

FIG. 13 illustrates the study design that compares whole genome expressed genes of subjects with heat stress and those with heat stroke as detected by a microarray.

FIGS. 14A-14E together describe the information shown by Table 2A.

FIGS. 15A to 15F together describe the information shown by Table 2B.

FIGS. 16A to 16CC together describe the information shown by Table 3A.

FIGS. 17A to 17KK together describe the information shown by Table 3B.

### DETAILED DESCRIPTION OF THE TECHNOLOGY

Exposure to excessive heat has wide ranging physiological impacts for all humans, often amplifying existing conditions and resulting in premature death and disability; see <hypertext transfer protocol secure:://www.who.int/news-room/fact-sheets/detail/climate-change-heat-and-health> (last accessed May 16, 2022).

Exertional heat injury and heatstroke occur in otherwise healthy younger individuals during vigorous exercise in hot or temperate environments; Bouchama, A, et al. Classic and exertional heatstroke. NATURE REVIEWS DISEASE PRIMERS 8, 8 (2022). It is usually observed in recreational and elite athletes, and military personnel and occupational workers. Exertional heatstroke differs from classic or nonexertional heatstroke, which results from passive exposure to high ambient temperatures often accompanied by high humidity and occurs in epidemic form during heatwaves, particularly among older individuals who often have pre-existing illnesses. Sonna, et al., J APPL PHYSIOL 96, 1943-53 (2004) described expression changes in RNA from peripheral blood mononuclear cells associated with exertional heatstroke in four soldiers. During exertional heatstroke elevations in HSPA1A, HSPA1B, HSPA6, DNAJB1, and HSPB1 were demonstrated. However, FKBP5 expression was found to be insignificantly downregulated. In athletes with a prior history of exertional heat illness, lymphocyte HSP72 response was reduced compared to controls; Ruell, P. A., et al. Plasma and lymphocyte Hsp72 responses to exercise in athletes with prior exertional heat illness; AMINO ACIDS, 2014, 46(6):1491.

Among the various embodiments of this disclosure a test for expression of heat shock or heat stress proteins is disclosed based on discovery of specific genetic markers. These markers comprise mRNAs encoding heat shock proteins that correlate with a subject having heatstroke compared to normal control subjects.

Methods disclosed herein allow one to distinguish heatstroke, such as nonexertional heatstroke, from other conditions characterized by fever or hyperthermia, e.g., alteration of the nervous system that may occur during an infection or drug toxicity and other non-heat-stroke medical conditions. Fever is typically associated with release of pyrogenic cytokines into the circulation after stimulation by a microorganism or other agent and/or reset of the thermoregulatory center in the hypothalamus from 37° C. (or normal body temperature) to a higher value such as 40° C., and/or muscular shivering that raises the core temperature). In hyperthermia, the thermoregulatory center is typically set at 37° C. and remains at this level. When a subject is exposed to excessive environmental or external heat, the thermoregulatory center strives to maintain this level of 37° C., although it may be overcome.

The methods disclosed herein may be used to distinguish patients with different types of heatstroke (e.g., nonexertional or classic vs. exertional), different degrees or severities of heatstroke, as well as distinguish patients having heatstroke from patients with less severe heat-related conditions. It may be used to longitudinally follow treatment of patients having heatstroke and assess effectiveness of a treatment.

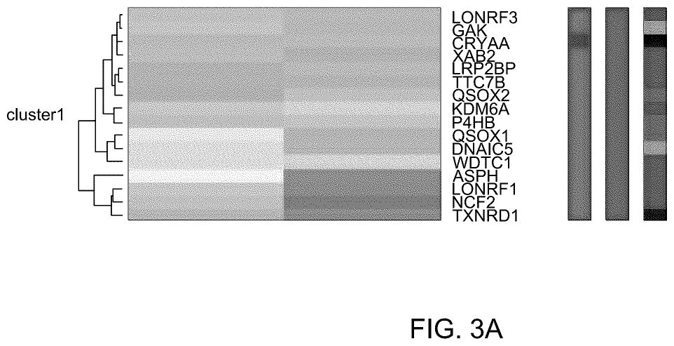

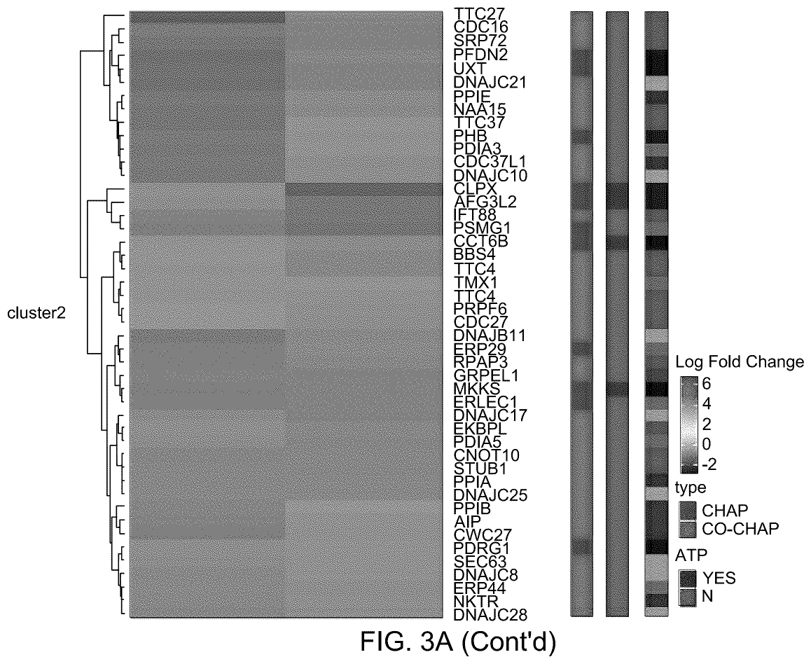

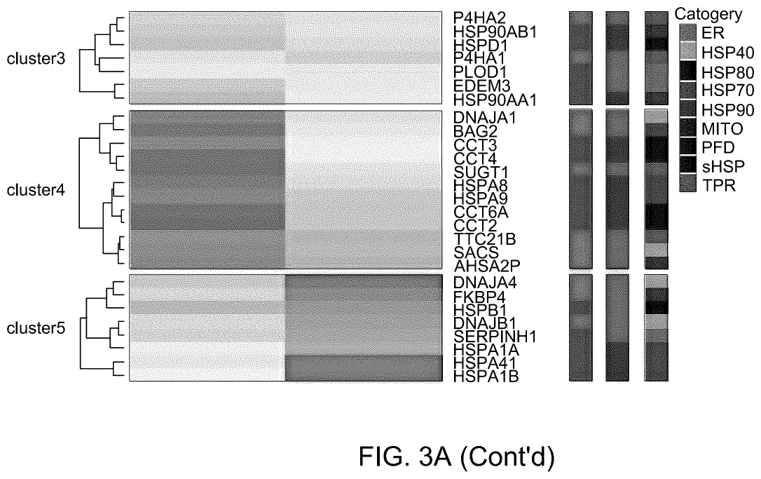

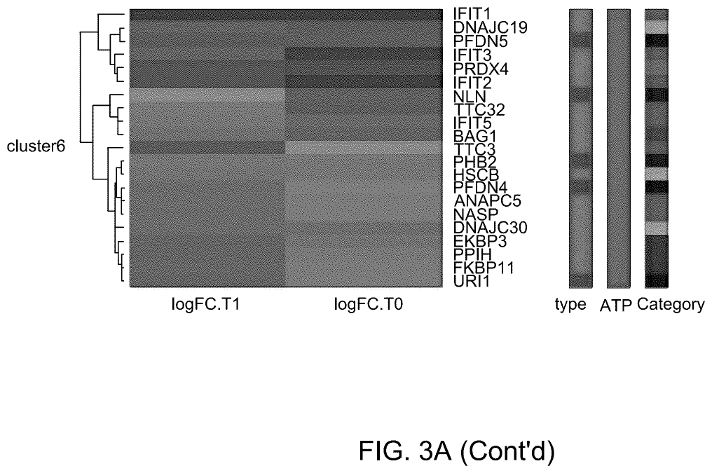

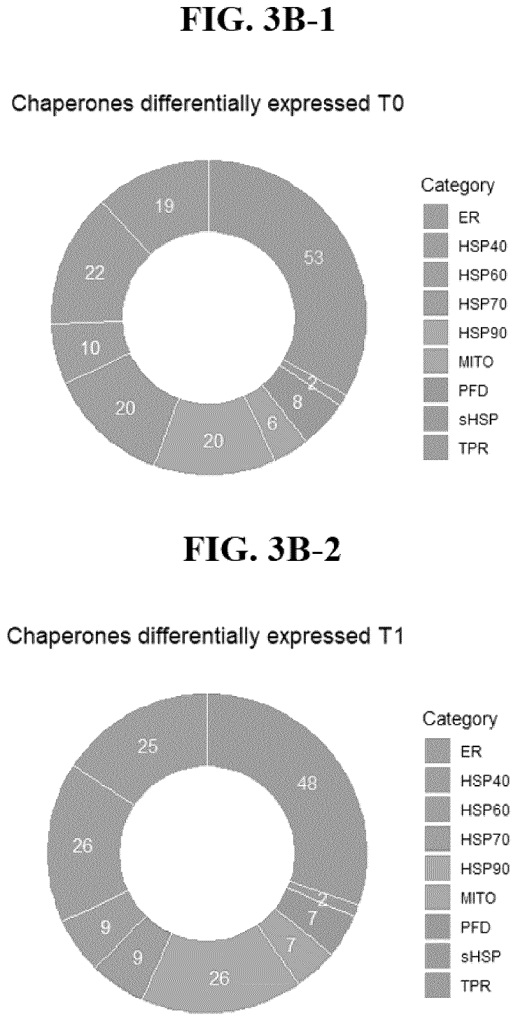

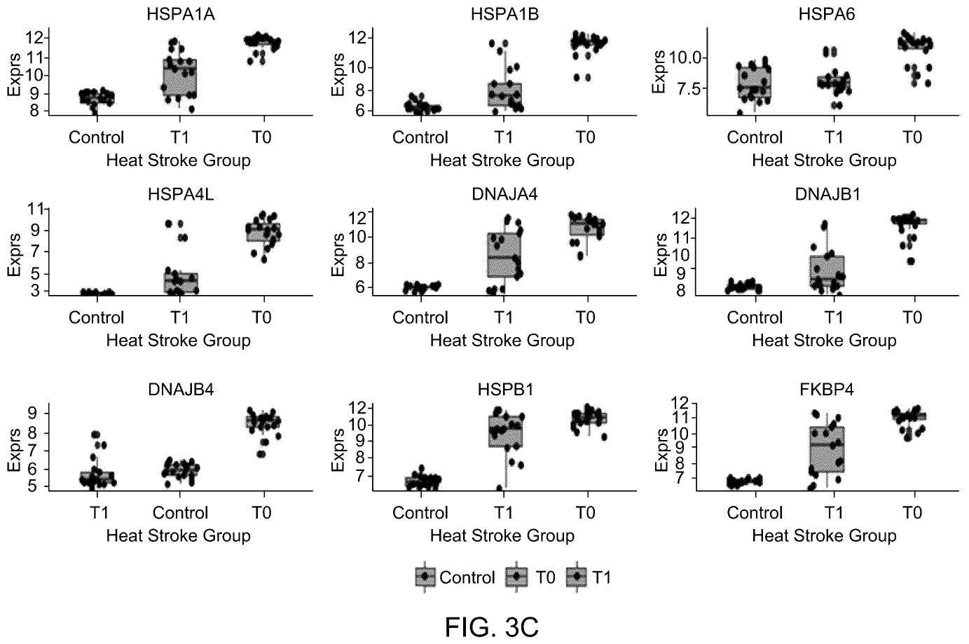

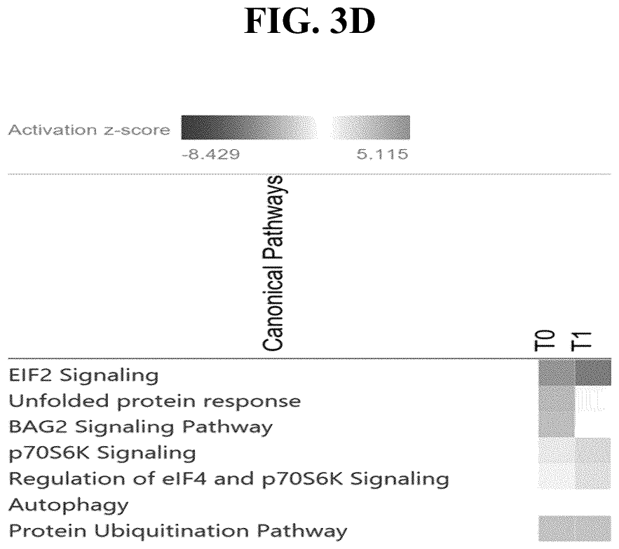

The present invention relates to methods for diagnosing or differentially diagnosing heatstroke by detecting altered expression of specific biomarkers that correlate with heatstroke. These markers include HSPA1A, HSPA1B, HSPA6, HSPA4L (HSP70 family); HSPB1 (heat shock family B); DNAJA4, DNAJB1, DNAJB4 (HSP40 family); or FKBP4 (FKBP Prolyl Isomerase 4). In some instances, a positive diagnosis is made when levels of particular RNA or protein markers differ from control values and in other instances a negative diagnosis is made when particular markers are absent or different markers are detected. As shown by FIG. 3C, heatstroke is characterized by elevations in the heatstroke biomarkers described herein.

The detection of HSP gene expression can be based on a multiplex real-time reverse transcription polymerase chain reaction (rRT-PCR) assay which targets two or more HSPs from the panel simultaneously. The diagnostic test of the invention may comprise, consist essentially of, or consist of a test that employs 1, 2, 3, 4, 5, 6, 7, 8, or 9 of HSPA1A, HSPA1B, HSPA6, HSPA4L (HSP70 family); HSPB1 (heat shock family B); DNAJA4, DNAJB1, DNAJB4 (HSP40 family); or FKBP4 (FKBP Prolyl Isomerase 4) as well as additional markers such as other members of the families described above or their functional analogs. In one embodiment, it may employ a single or multiple markers from the HSP70 family, and/or a single or multiple markers of heat shock family B, and/or FKBP4.

Other technology which may be used to implement the invention or used in conjunction with the invention are incorporated by reference to U.S. Pat. Nos. 10,307,287, 10,188,548, and to <hypertext transfer protocol secure://innovations.kaimrc.med.sa/en/feature/381/a-smart-way-to-heat-the-heat> (last accessed May 16, 2022). Various heat-related conditions are also described by and incorporated by reference to the documents cited above.

All nine biomarkers disclosed herein are positively correlated with non-exertional heat stroke. However, these biomarkers can be positive in other heat-related conditions that are not heat stroke but at a much lower magnitude (see Table 1 below). These include heat stress, heat exhaustion, and hyperthermia-related to medical conditions (thyrotoxicosis) or recreational drugs (amphetamines, cocaine). The diagnosis of heat stroke can be based on the magnitude of increase of the nine HSP biomarkers. The Table 1 below shows the fold change of the nine HSP biomarkers compared to non-heat stroke subjects is significant. The minimum observed is 2.852-fold increase compared to heat-stressed control. In some embodiments, a two-fold or three-fold or more increase from the control values of any or combined biomarkers is considered positive for the diagnosis of heat stroke. Table 1:

TABLE 1
Heat shock protein gene expression in patients with heat stroke relative to heat stressed control

| | Expr Log: | Expr Fold | Adjusted | Expr Log: | Expr Fold | Adjusted |
|---|---|---|---|---|---|---|
| ID | Ratio* T0 | Change** T0 | p-value | Ratio* T1 | Change T1 | p-value T1 |
| HSPA1A | 3.146 | 8.891321515 | 1.0918E−22 | 1.509 | 2.827306913 | 7.26E−05 |
| HSPA1B | 5.157 | 34.97894958 | 6.01577E−23 | 1.345 | 2.524437148 | 0.006220856 |
| HSPA6 | 2.852 | 8.557606683 | 3.84432E−07 | NS | NS | NS |
| HSPA4L | 5.618 | 39.94998785 | 8.12273E−14 | 1.71 | 2.69370857 | 0.039307201 |
| HSPB1 | 3.727 | 12.60526524 | 1.83082E−22 | 2.817 | 6.707924711 | 3.14091E−08 |
| DNAJA4 | 4.597 | 23.19026009 | 1.42037E−18 | 2.455 | 5.247242361 | 0.000104618 |
| DNAJB1 | 3.493 | 11.27924248 | 2.06734E−20 | 0.915 | 1.882905155 | 0.007782605 |
| DNAJB4 | 3.224 | 8.576958757 | 1.88472E−13 | NS | NS | NS |
| FKBP4 | 4.145 | 16.66952192 | 4.01203E−23 | 2.096 | 4.028666951 | 9.09808E−05 |

*Values are changes (in fold log₂ scale) in gene expression immediately after heat stroke (T0) and 4 ± 3 hours after heat stroke (T1), relative to control, heat stressed.
**Numerical expression before transformation to log₂ scale.
The comparison was made between heat stroke (T0 and T1) with heat-stressed control using a Generalized mixed linear model.
Adjusted P-value using Benjamini-Hochberg method.
NS = not statistically significant.

Heat stress response, Heat shock, and heatstroke. For most living species including humans the universal host response to extreme heat is associated with expression of heat shock or heat stress proteins. Past studies were based on an in-vitro heat shock cellular model and demonstrated an increase and not a decrease of the family of heat-inducible HSPs, including HSPA1A and HSPA1B. Heat shock proteins are often expressed or induced at different levels in different tissues. Hyperthermia is a hallmark of heatstroke, as indicated by the body core temperature exceeding 42° C. in 37% of the patients. Bouchama A, et al., Heatstroke. THE NEW ENGLAND JOURNAL OF MEDICINE, 346(25):1978-1988 (2002); Leon L R, et al., Heatstroke. COMPREHENSIVE PHYSIOLOGY, 5(2):611-647 (2015). This level of hyperthermia is known to induce heat shock to human cell culture grown in-vitro, in organismal models, or in mammals, which may result in macromolecular damage, including to proteins, membrane lipids, and DNA.

The Centers for Disease Control describe a number of heat-related conditions which the methods disclosed herein help diagnose, exclude or monitor. For example, the detection of mRNA biomarkers as described herein, when correlated with heatstroke, can help differentially diagnose the heat-related condition and help select treatments. The diagnosis or identification of a heat-related condition, such as heatstroke, using the biomarkers disclosed herein may further comprise medical evaluation of a subject for symptoms of the heat related condition.

Heatstroke is the one of the most serious heat-related illnesses. It occurs when the body can no longer control its temperature: the body's temperature rises rapidly, the sweating mechanism fails, and the body is unable to cool down. When heatstroke occurs, the body temperature can rise to 106° F. or higher within 10 to 15 minutes. Heatstroke can cause permanent disability or death if the person does not receive emergency treatment. Symptoms of heatstroke include confusion, altered mental status, slurred speech, loss of consciousness coma), hot, dry skin or profuse sweating, seizures, and very high body temperature. Heatstroke can be fatal if treatment is delayed. Initial treatments include contacting emergency care, remaining with the heatstroke subject until emergency medical services are available, moving the subject to a shaded, cool area and removing outer clothing, cooling the subject quickly using cold water or an ice bath, wetting the skin, placing a wet cloth on the skin of the subject, for example, on head, neck, armpits, and groin, and/or soaking subject's clothing with cool water. Likewise, air may be circulated around the subject.

Exertional Heatstroke (EHS), Non-Exertional Heatstroke (NEHS), and other heat-associated disorders are classified in different ways. Heatstroke may be classified as exertional heatstroke (EHS), which is due to overexertion in hot or even temperate weather; or non-exertional heatstroke (NEHS), which occurs in climactic extremes and affects the elderly, infants, and chronically ill.

Heat exhaustion is the body's response to an excessive loss of water and salt, usually through excessive sweating. Heat exhaustion is most likely to affect the elderly, people with high blood pressure, or those working in a hot environment. Symptoms of heat exhaustion include headache, nausea, dizziness, weakness, irritability, thirst, heavy sweating, elevated body temperature, and/or decreased urine output. Initial treatment of a subject who has heat exhaustion may include: contacting emergency care, or taking the subject to a clinic or emergency room for medical evaluation and treatment, having someone stay with the subject until first aid or medical help is available, removing the subject from the hot area and giving liquids to drink, removing unnecessary clothing, including shoes and socks, cooling the subject the with cold compresses or having the subject wash their head, face, and neck with cold water, further encouraging subject to take frequent sips of cool water or electrolyte solutions is advisable.

Rhabdomyolysis (rhabdo) is a medical condition associated with heat stress and prolonged physical exertion. Rhabdo causes the rapid breakdown, rupture, and death of muscle. When muscle tissue dies, electrolytes and large proteins are released into the bloodstream. This can cause irregular heart rhythms, seizures, and damage to the kidneys. Symptoms of rhabdo include muscle cramps/pain, abnormally dark (tea or cola-colored) urine, weakness, or exercise intolerance; in some cases rhabdo is asymptomatic. Subjects with symptoms of rhabdo should cease activity, drink more liquids (water preferred), and/or seek immediate care at a medical facility where the subject can be further evaluated for rhadbo, for example, by analysis of the subject's blood for creatine kinase. Detection of biomarkers associated with severe heat-related conditions, such as heatstroke, help differentially diagnose a patient exhibiting features of rhabdo from other heat-associated conditions.

Heat syncope is a fainting (syncope) episode or dizziness that usually occurs when standing for too long or suddenly standing up after sitting or lying. Factors that may contribute to heat syncope include dehydration and lack of acclimatization. Symptoms of heat syncope include fainting (short duration), dizziness, light-headedness from standing too long or suddenly rising from a sitting or lying position. Treatments include sitting or lying down in a cool place, slowly consuming water, clear juice or a sports drink. Detection of biomarkers associated with more severe heat-related conditions than heat syncope, such as heatstroke, help differentially diagnose a patient exhibiting features of a heat-associated condition.

Heat cramps usually affect subjects who sweat a lot during strenuous activity. This sweating depletes the body's salt and moisture levels. Low salt levels in muscles cause painful cramps. Heat cramps may also be a symptom of heat exhaustion. Symptoms include muscle cramps, pain, or spasms in the abdomen, arms, or legs. Treatments include drinking water or consuming a snack or a drink that replaces carbohydrates and electrolytes (such as sports drinks) every 15 to 20 minutes and avoiding salt tablets. Medical help should be obtained if the subject has heart problems, is on a low sodium diet or has cramps that do not subside within an hour. Detection of biomarkers associated with more severe heat-related conditions than heat cramps, such as heatstroke, help differentially diagnose a patient exhibiting features of a heat-associated condition.

Heat rash is a skin irritation caused by excessive sweating during hot, humid weather. Symptoms of heat rash include red clusters of pimples or small blisters which usually appear on the neck, upper chest, groin, under the breasts, and in elbow creases. Initial treatments include working in a cooler, less humid environment, keeping the rash area dry, applying powder to decrease friction and for comfort and avoiding the use of ointments or creams. Detection of biomarkers associated with more severe heat-related conditions than heat rash, such as heatstroke, help differentially diagnose a patient exhibiting features of a heat-associated condition.

Classic (non-exertional) heat stroke often occurs during an episode of heat waves, i.e., when ambient temperatures are higher than the historical average of an area. The definition of heat waves varies across countries because each population has a different level of adaptation and tolerability to heat. Currently, there is no standard method to define a heatwave. Likewise, there is no standard weather temperatures index to express extreme environmental heat. In the USA, heat waves definition is based on the heat index (the actual temperature adjusted for humidity), while most countries use air temperatures (dry-bulb temperatures) or, more recently, wet-bulb temperatures (a combination of air temperature and humidity). The CDC USA defines a heatwave as two or more consecutive days in which the daily minimum apparent temperature (heat index) in a particular city exceeds the 85th percentile of historical temperatures for that city. In Europe, the WHO considers a heatwave as a period in which the maximum and minimum apparent temperatures exceed the 90th percentile of the monthly distribution for at least two days. Perhaps, for our invention, we may refer to heat waves rather than giving a precise range of heat index, which does not apply to the rest of the world. Exertional heat stroke can occur even when ambient temperatures are not high.

Heat index (typically used in the United States), also known as the apparent temperature, is what the temperature feels like to the human body when relative humidity is combined with the air temperature. This has important considerations for the human body's comfort. When the body gets too hot, it begins to perspire or sweat to cool itself off. If the perspiration is not able to evaporate, the body cannot regulate its temperature. The heat index may be calculated based on the formulas described by, and incorporated by reference to Steadman, R. G. The Assessment of Sultriness Part I: A Temperature-Humidity Index Based on Human Physiology and Clothing Science. JOURNAL OF APPLIED METEOROLOGY. 1979, 18 (7): 861-873 or Steadman, R. G. The Assessment of Sultriness. Part II Effects of Wind, Extra Radiation and Barometric Pressure on Apparent Temperature, JOURNAL OF APPLIED METEOROLOGY. 1979, 18 (7): 874-885.

Alternatively, as used in some countries, a dry- or wet-bulb temperature is used to define what is considered a heat wave or elevated environmental heat. Wet-bulb temperature is literally what a thermometer measures if a wet cloth is wrapped around it. The temperature in a typical weather forecast is technically a dry-bulb temperature, since it is measured with a dry thermometer. Wet-bulb temperature can estimate what skin temperature would be for an individual undergoing perspiration and is often used to approximate how people may fare in extreme heat. In some embodiments of the methods disclosed herein, the biomarkers are isolated from a subject exposed to an environment having a dry bulb temperature of ≥32, 33, 34, 35, 36, 37, 38, 39 or 40° C.

A comfortable wet bulb temperature is considered to be about 22° C. (70° F.) and a limit of human endurance as measured by a wet bulb temperature has been considered to be about 35° C. (95° F.) <hypertext transfer protocol secure//www.technologyreview.com/2021/07/10/1028172/climate-change-human-body-extreme-heat-survival> (last accessed Mar. 24, 2023, incorporated by reference). In some embodiments of the methods disclosed herein, the biomarkers are isolated from a subject exposed to an environment having a wet bulb temperature of ≥22, 23, 24, 25, 26, 27, 28, 29, 30, 31, 32, 33, 34 or 35° C.

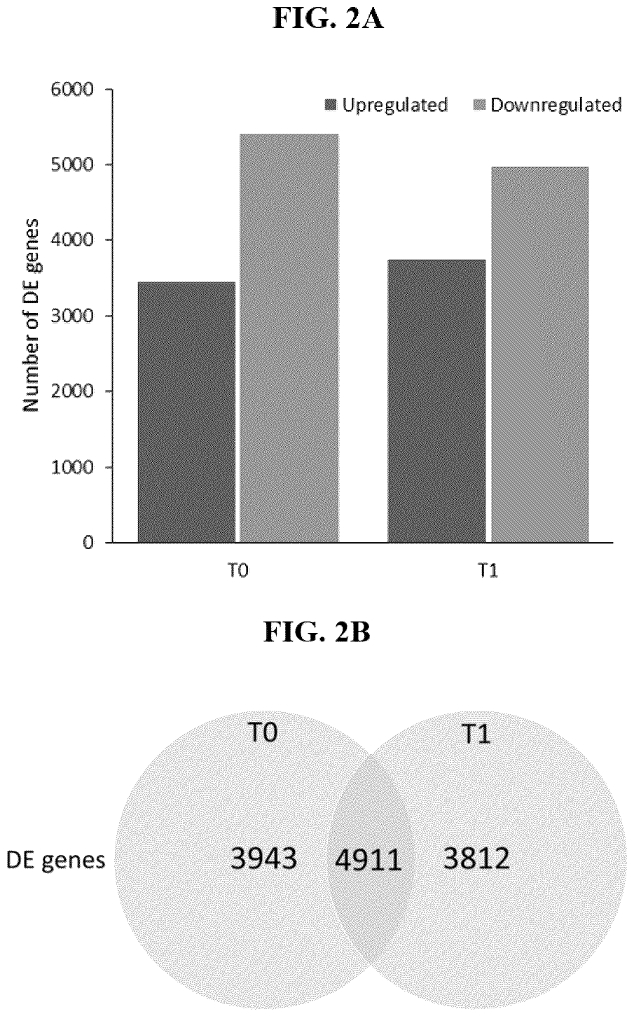

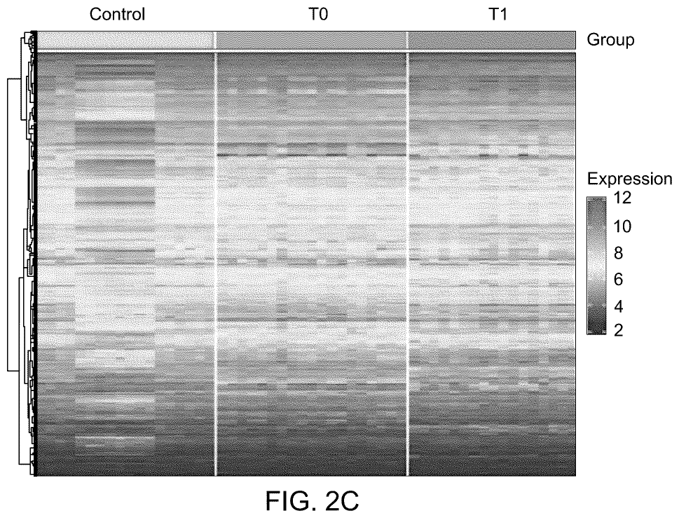

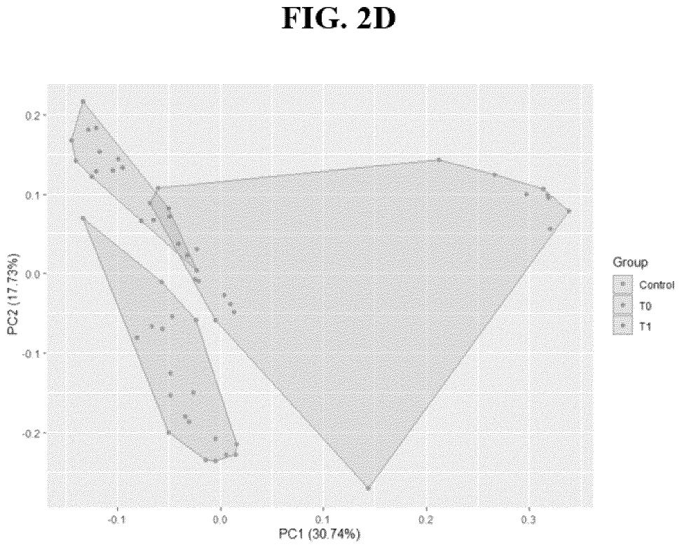

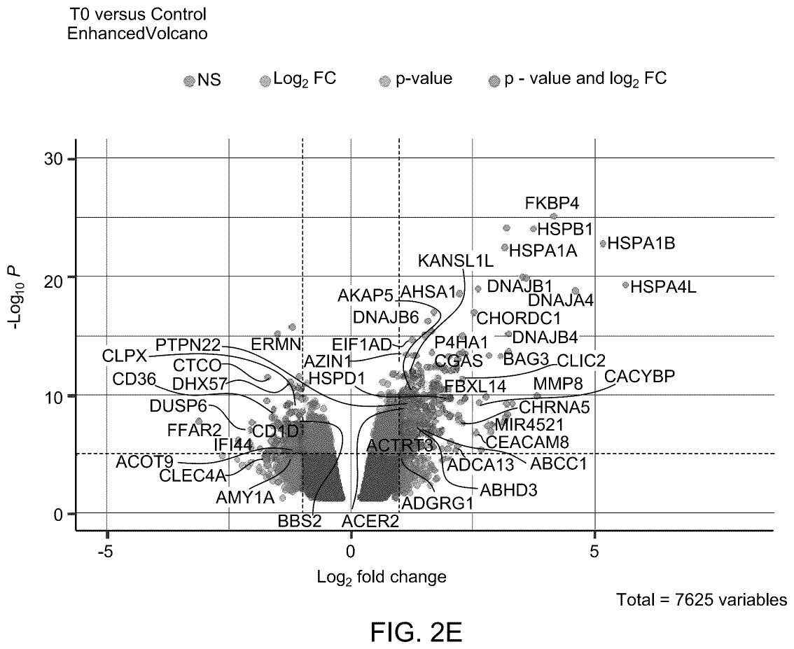

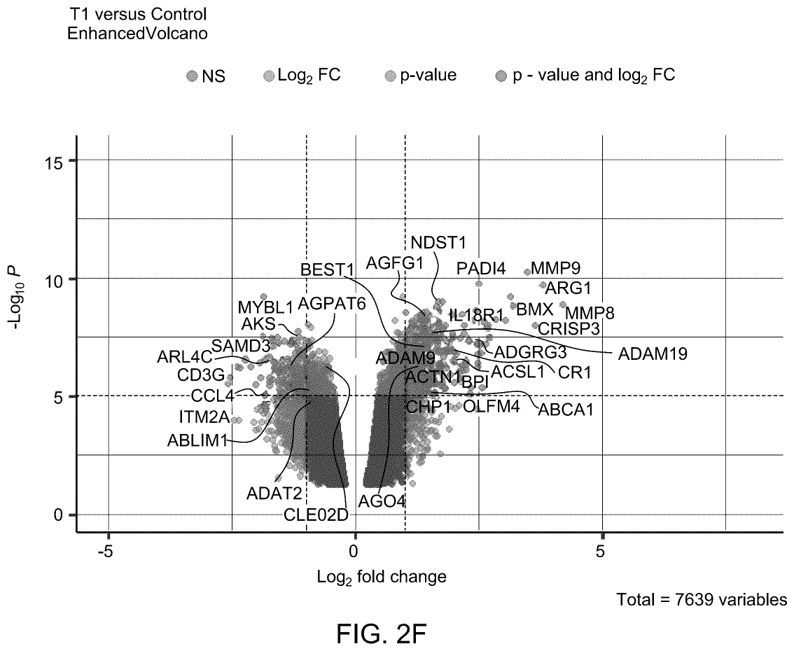

RNA Biomarkers refer to RNAs that encode all or part of a protein, such as heat shock or heat stress proteins or other protein associated with heatstroke or heat-related conditions. Pre- and post-cooling biomarkers are described herein, for example, in FIGS. 2E and 3C. Some preferred RNA biomarkers are mRNAs that encode HSP A1A, HSP A B, HSPA6, HSPA4L, DNAJA4, DNAJB1, DNAJB4, HSPB1, and FKBP4.

Biological sample. This term includes but is not limited to a biological fluid such as blood, plasma or serum, saliva, mucosal secretions, or urine, a solid biological sample such as a tissue biopsy, and the like. Samples may be fresh, frozen, preserved, such as in archival paraffin-embedded tissue. Preferred samples include bodily fluids particularly buffy coat cells from blood. Samples may be processed to concentrate or isolate biomarkers or to eliminate contaminating nucleic acids, proteins or other materials. The invention may be practiced using samples from distinct tissues including blood, CSF, epidermal tissue, intestinal tissue, kidney, liver, heart, spleen, lymphatic, mucosal, and other tissues.

Table 2 below displays the accession numbers describing the RNA sequences of each biomarker. Please note that some genes have several isomers (DNAJA4 has 8).

TABLE 2

| Gene ID | Accession Number | Gene ID | Accession Number |
|---|---|---|---|
| HSPA1A | NM_005345.6 | DNAJA4_6 | NM_001130186.2 |
| HSPA1B | NM_005346.6 | DNAJA4_7 | NM_001130187.2 |
| HSPA6 | NM_002155.5 | DNAJA4_8 | NM_001130188.2 |
| HSPA4L_1 | NM_014278.4 | DNAJB1_1 | NM_006145.3 |
| HSPA4L_2 | NM_001317381.2 | DNAJB1 2 | NM 001300914.2 |
| HSPA4L_3 | NM 001317382.2 | DNAJB1 3 | NM 001313964.2 |
| HSPA4L_4 | NM_001317383.2 | DNAJB4_1 | NM_007034.5 |
| HSPB1 | NM_001540.5 | DNAJB4_2 | NM_001317099.2 |
| DNAJA4_1 | NM_018602.4 | DNAJB4 3 | NM 001317100.2 |
| DNAJA4_2 | NM_001130182.2 | DNAJB4_4 | NM_001317101.2 |
| DNAJA4_3 | NM_001130183.2 | DNAJB4_5 | NM_001317102.2 |
| DNAJA4 4 | NM 001130184.2 | DNAJB4_6 | NM_001317103.2 |
| DNAJA4_5 | NM_001130185.2 | FKBP4 | NM 002014.4 |

Identification and measurement of heat shock or heat-associated proteins. HSPs and heat-associated proteins as well as other kinds of cellular proteins may be detected by methods known in the art including by sandwich immunoassays or by ELISAs. Immunoassays rely on antibodies generated by immunizing animals such as goats and rabbits with a representative antigen harvest from corresponding null cell lines. Such methods are known in the art and are incorporated by reference to Xiaoihui, L., et al. Identification and Quantification of Heat-Shock Protein 70: A Major Host-Cell Protein Contaminant from HEK Host Cells; BIOPROCESS TECH. Oct. 1, 2015, 13; Wang X, et al., Host Cell Proteins in Biologics Development: Identification, Quantitation, and Risk Assessment. BIOTECHNOL. BIOENG. 103(3) 2009: 446-458; Zhu-Shimoni J. et al. Host Cell Protein Testing By ELISAs and the Use of Orthogonal Methods, BIOTECHNOL. BIOENG. 111(12) 2014: 2367-2379. One or more of these methods may be used to identify or characterize the heat shock and heat-associated proteins disclosed herein.

Detection methods that are preferred in the context of the present disclosure determine the level of said at least one biomarker in a sample by a detection method selected from the group consisting of microarray, RNA sequencing, PCR, multiplex-PCR, western blot, mass spectrometry, mass spectrometry immunoassay (MSIA), antibody-based protein chips, 2-dimensional gel electrophoresis, high-performance liquid chromatography, (HPLC), cytometry bead array (CBA), protein immunoprecipitation, radioimmunoassay, ligand binding assay, and enzyme-linked immunosorbent assay (ELISA). Heat shock or heat stress proteins or other biomarkers released into the circulation may be assayed from blood, plasma, or serum samples. In some embodiments, a microarray assay, such as an assay using a GeneChip or Affymetrix microarray, is used to detect the RNA biomarkers disclosed herein. Such microarrays are commercially available.

In some embodiments, expression of a biomarker as disclosed herein in a subject having heatstroke, for example for RNA encoding HSP A 1A, HSP A 1 B, HSPA6, HSPA4L, DNAJA4, DNAJB1, DNAJB4, HSPB1, and/or FKBP4 may be increased by >0, 1, 2, 3, 4, 5, 6, 7, 8, 9, 10, 15, 20, 25, 30, 35, 40, 45, 50 or more times (increase in fold) compared to control values, such as those from heat-stressed control. Similar decreases in RNA biomarkers elevated during heatstroke accompany treatment of a heatstroke patient by cool or other means.

Microarray technology involves placing thousands of gene sequences in known locations on a glass slide called a gene chip. A sample containing DNA or RNA is placed in contact with the gene chip. Complementary base pairing between the sample and the gene sequences on the chip produces light that is measured. Microarrays and methods using them are further described by, and incorporated by reference to GeneChip® Microarray, Structure & Function of GeneChip Microarrays at <hypertext transfer protocol secure://www.csus.edu/indiv/r/rogersa/bio181/genechipho.pdf> or to <hypertext transfer protocol secure://en.wikipedia.org/wiki/DNA_microarray> (last accessed Dec. 16, 2022, incorporated by reference). Such arrays may be employed to identify the biomarkers disclosed herein.

Multiplex methods may be employed to detect more than one biomarker at a time. Such means for quantifying is, for example, the simultaneous amplification of more than one target sequence in a single reaction tube using more than one primer pair. Such multiplex-PCR is known in the art and commercially available.

Determination of differential Levels of Biomarkers. A differential level of one or more heat shock protein or co-chaperone biomarkers in a biological sample from the subject, compared to a healthy control or reference value, may be used as a basis for identifying the presence of heatstroke in the subject. Biomarkers include but are not limited to RNA encoding heat shock proteins or co-chaperone proteins, or heat shock protein or co-chaperone levels. Specific biomarkers identified and evaluated be the inventors include HSPA1A, HSPA1B, HSPA6, HSPA4L, DNAJA4, DNAJB1, DNAJB4, HSPB1, and/or FKBP4.

Levels of one or more than one biomarker may be measured. However, the methods disclosed herein are not restricted to any particular method for determining the level of a given biomarker and encompass all means that allow for quantification, or estimation, of the level of said biomarkers, either directly or indirectly.

The term “measuring the expression level of” a biomarker in a sample, control, or reference, as described herein, refers to the quantification of biomarkers indirectly via assessing the gene expression of the encoding gene of the biomarker, for example, in some embodiments by quantifying the expressed mRNA encoding for the respective biomarker in the tested sample.

In other embodiments concentration(s) of the biomarkers in said samples may be directly quantified via measuring the amount of protein present in the tested sample.

Detection kits. A kit for detection of alterations of mRNA or protein biomarkers for the heat shock proteins and other heat-associated proteins disclosed herein preferably comprises probes or primers that detect nucleic acids encoding HSPA1A, HSPA1B, HSPA6, HSPA4L, DNAJA4, DNAJB1, DNAJB4, HSPB1, and/or FKBP4; or, alternatively, antibodies that bind to these proteins.

Preferably, a diagnostic multiplex-PCR kit is provided which quantifies the expression levels of the above biomarkers. Quantifying may proceed by simultaneous amplification of two or more RNA biomarkers in a single reaction tube using more than one primer pair. Such multiplex PCR is known and commercially available. Microarrays as disclosed herein may be incorporated into a kit.

In other embodiments, a kit may contain probes or primers that bind to or amplify mRNA encoding these proteins. In preferred embodiments, the kit may contain a microarray of human DNA suitable for detecting RNA encoding human heat shock or heat stress proteins.

In an alternative embodiment, a kit may contain antibodies that specifically bind to the heat shock and other heat-associated proteins disclosed herein, such as antibodies or other ligands binding to HSP A1A, HSPA1B, HSPA6, HSPA4L, DNAJA4, DNAJB1DNAJB4, HSPB1, or FKBP4.

Kits may be supplied with instructional materials. Instructions may be printed on paper or other substrates, and/or may be supplied as an electronic-readable medium, such as a floppy disc, CD-ROM, DVD-ROM, zip disc, videotape, audio tape, or other readable memory storage device. Detailed instructions are not necessarily physically associated with the kit; instead, a user may be directed to an internet web site specified by the manufacturer or distributor of the kit or supplied as electronic mail.

### EXAMPLE

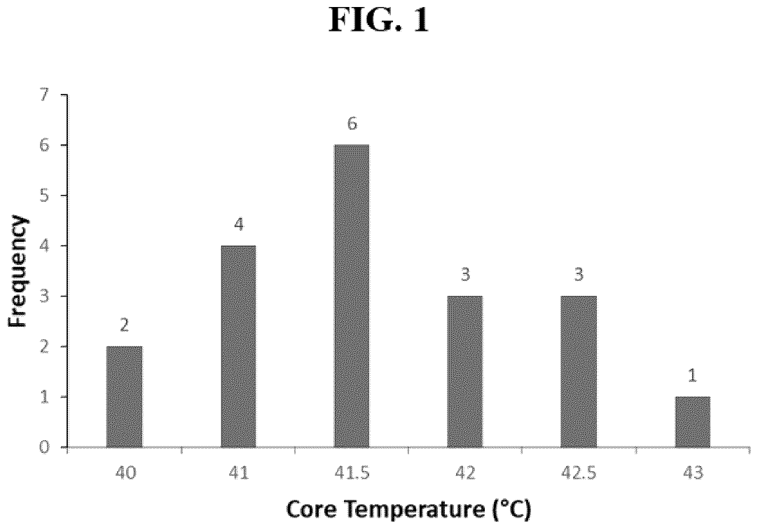

As disclosed herein, the inventors examined whether genome-wide transcriptional profiling could identify signature genes and critical pathways that distinguish subjects exposed to extreme environmental heat who progressed to heatstroke versus from those who did not. Gene expression profiling using a microarray assay, such as a GeneChip microarray, was performed on peripheral blood mononuclear cells (PBMC) from heatstroke patients (n=19) pre- and post-cooling; see FIG. 1 which describes core temperatures of 19 heatstroke patients upon admission and presentation to the hospital before cooling (T0). Age- and ethnicity-matched heat-stressed subjects (n=19) exposed to the same extreme environmental heat were used as a control group.

All pilgrims were exposed to an ambient temperature and humidity in Makkah during the 5-day pilgrimage (30 Aug. to 3 Sep. 2017) between 37 to 43° C. and a relative humidity of 35 to 49% (enclose graphic of the study design). Because of the strict religious rituals, all pilgrims, including our study population, i.e., heat stroke patients and heat-stressed participants stayed within well-defined geographical boundaries, wore similar white clothing, ate the same food, and moved together. Therefore, they received the same dose of heat, experiencing near-experimental conditions of severe heat stress

The patients with heatstroke had a mean rectal temperature on admission of 41.7±0.8° C., and eight were in a deep coma (Glasgow Coma Score=3).

The transcriptomic analysis revealed that heat shock proteins (HSPs), co-chaperones, and chaperonins genes were the most significantly expressed genes with the highest fold-change, consistent with a robust heat shock response.

Other key pathways included the unfolded protein response, mitochondrial dysfunction, oxidative stress, DNA damage response, and immune response, indicating severe proteostasis disturbance, alteration of bioenergetics, DNA integrity, and immunity.

Cooling therapy attenuated these alterations without complete restoration of homeostasis.

The significantly expressed genes were validated by a real-time polymerase chain reaction.

These results identified the gene signatures of heatstroke and suggest bioenergy failure and proteotoxicity as pathogenic mechanisms of heatstroke.

RNAs described herein that increase or decrease in subjects having heatstroke or other heat-associated conditions compared to the levels of the same RNAs in a subject not having heatstroke or heat-associated conditions may be employed to further diagnose whether a subject has a particular heat-associated condition. Similarly, such RNAs whose expression or levels correlate with heat-associated conditions may be employed to determine the effects of such heat-associated conditions on the biological pathways disclosed herein or in the Supplemental Tables and where necessary provide therapy that compensates for deleterious effects of the heat associated condition or that promotes favorable effects of the increase or decrease in levels of particular RNA or protein biomarkers.

Specific materials and methods and results obtained therefore are described in the following Example. References cited in the Example, which describe protocols and materials, are incorporated by reference.

Additional data obtained and useful for distinguishing subjects having heatstroke or related conditions are disclosed by Supplementary Tables 2A, 2B, 3A and 3B which are appended to the end of, and form a part of, this disclosure.

### Materials and Methods

Genome-wide transcriptional profiling reveals the molecular signature of heatstroke.

Heatstroke patients. Following approval by the Institutional Review Board of King Abdullah International Medical Research Center (KAIMRC), this study was conducted at the Mina Emergency Hospital, Mecca, Saudi Arabia, during the pilgrimage of August 2017. Because heatstroke patients present with severe alteration of the level of consciousness, written consent was obtained from the legal representatives upon admission. Subsequently the consent by the patients who improved post-cooling was confirmed. Written informed consent was obtained from all control subjects. All procedures were performed following Helsinki's World medical association declaration on ethical principles for medical research involving human subjects. Nineteen consecutive adult patients with a rectal temperature>40.1° C., associated neurologic alterations (including delirium, convulsions, or coma), and high environmental temperature and humidity exposure were enrolled. Rectal temperature, blood pressure, pulse, and respiratory rates were obtained immediately on admission. Neurological status was obtained by the Glasgow come score (GCS). Patients who presented in cardiac attest were excluded as well as patients who declined to sign a written consent to participate in the study.

Control subjects. Nineteen pilgrims, friends, or relatives of heatstroke patients living in the same environmental heat were used as a control group. The control subjects were age- and ethnicity-matched to the study group. Vital signs and medical history were recorded.

Blood Collection. Blood samples were obtained from the control subjects upon enrollment and from heatstroke patients on arrival to the cooling unit, precooling (T0), and post-cooling (T1). Blood was drawn by venipuncture into sterile, BD Vacutainer® EDTA tubes (BD Biosciences, USA) at each time point.

A complete blood count, liver, renal and cardiac profile, and creatine phosphokinase activity (CPK) was measured immediately at the hospital laboratory as part of patient care. In addition, the peripheral blood mononuclear cells (PBMC) and plasma were separated and snap-frozen immediately in liquid nitrogen. Afterward, the samples were stored at −80° C. before transportation in dry ice to the laboratory at KAIMRC, Riyadh for further analysis.

PBMC isolation, PBMCs were isolated using Leucosep™ tubes (Greiner Bio-One, Frickenhausen, Germany) according to the manufacturer's instructions. Briefly, the separation medium was prelayed into the bottom of the Leucosep™ tubes through the porous barrier. 15 ml of blood was carefully poured into the Leucosep™ tubes and centrifuged for 1000×g for 10 min at room temperature. The enriched PBMC layer was collected into a new 50 centrifuge tube. The red blood cells were lysed in 1×RBC lysing buffer, then washed the PBMC twice in 1×PBS with 1% FBS (300×g for 10 min). All PBMC samples were aliquoted, snap-frozen them in liquid nitrogen, and then stored immediately at −80° C.

RNA extraction. According to the manufacturer's protocol, the total RNA was extracted from the PBMCs using TRIzol Reagent (Invitrogen) and SV Total RNA Isolation System (Promega, USA). The RNA was quantified and tested its integrity and quality using the NanoDrop ND-2000 spectrophotometer (NanoDrop Technologies) and the bioanalyzer RNA 6000 nanochip on Agilent 2100 Bioanalyzer (Agilent Technologies, Santa Clara, CA, USA), respectively, following the manufacturer's protocol.

Microarray Analysis. The gene expression was performed using human Clariom D™ arrays from Affymetrix (Thermo Fisher Scientific, Waltham MA, USA). Following amplification, labeling, and hybridization of 250 ng of the total RNA, cDNA was prepared and labeled the fragmented cDNA with biotin using GeneChip® WT Plus Reagent Kit. d approximately 5.5 μg of biotinylated cDNA was combined to the human Clariom D arrays in Affymetrix GeneChip hybridization oven 645 at 45° C. with rotation at 60 rpm for 16 hours. The arrays were then washed and strained using Affymetrix GeneChip Fluidics Station 450. Subsequently, GeneChip Scanner 3000 7G was used to scan the chip arrays. Finally, the raw data on CEL files were submitted to bioinformatics for analysis.

Quality control, pre-processing, and normalization of microarray data. The quality of the CEL files was assessed for each time-point using the Bioconductor array Quality Metrics program; Kauffmann A, et al., arrayQualityMetrics—a bioconductor package for quality assessment of microarray data. BIOINFORMATICS, 2009, 25(3):415-416. In particular, the program for outlier detection in microarray plots and differences was used between arrays at all time points. The raw CEL files were processed using freely available updated chip definition files for Clariom D Human array based on Entrez genes, which remaps every probe in the array, re-aligning to the sequences of the latest genome assembly. Specifically, three libraries of version 22 were used, namely, clariomdhumanhsentrezgcdf, clariomdumanhsentrezgprobe, and clariomdhumanhsentrezg. Db; Dai M, et al: Evolving gene/transcript definitions significantly alter the interpretation of GeneChip data. NUCLEIC ACIDS RESEARCH, 2005, 33(20):e175-e175. The raw CEL files were background-corrected, normalized using the Robust Multi-array Average method, and converted into numerical expressions using the Affymetrix package Bioconductor; Gautier L, et al., affyanalysis of Affymetrix GeneChip data at the probe level. BIOINFORMATICS 2004, 20(3):307-315.

Identification of differentially expressed genes and statistical significance. The real-valued expression profiles were analysed in two phases to determine the genes underlying the host's response to environmental heat stress. In the first phase, the entire gene set was filtered using the variance method's adaptive gamma mixture model. Briefly, the variance of its average expression level between paired time points for each gene was calculated. A gamma mixture model with two components was fixed in the second phase and the genes with the highest posterior value were selected. The differentially expressed genes were performed using the Statistical analysis of the Microarrays tool; Tusher V G, et al., Significance analysis of microarrays applied to the ionizing radiation response. PROCEEDINGS OF THE NATIONAL ACADEMY OF SCIENCES 2001, 98(9):5116-5121.

Pathways, upstream and downstream effects analysis. IPA software (worldwide web.qiagen.com/ingenuity) was used to analyze the differentially expressed (DE) genes at T0 and T1 for the heatstroke patients and heat-stressed control subjects. IPA is a web-based application that enables the interpretation and significance of the differentially (DE) genes by analyzing their association with metabolic and signaling canonical pathways, predicting their upstream regulators and downstream biological functions. IPA uses Fisher's exact test and calculates a p-value for each category. A p-value<0.05 indicates a statistical significance. IPA estimates an activation Z-score to infer the likely activation state of the canonical pathway, upstream regulator, and biological function.

Quantitative real-time PCR (qRT-PCR). A total of 2 μg RNA in a 50 μl reaction was reverse-transcribed using the High-Capacity cDNA Reverse Transcription Kit, following the recommended instructions by the company (Applied Biosystems, USA). According to the manufacturer's instructions, qRT-PCR gene expression analysis was performed using SYBR™ Green PCR Master Mix (Applied Biosystems, USA). Three replicates were analyzed using randomly selected genes from the DE genes from the microarray results. The gene-specific oligonucleotides were synthesized by Macrogen. The forward and reverse sequences of the primers are included in Supplementary Table 1 at the end of the specification.

GAPDH was used as the endogenous control for all gene expression analyses. The analysis was performed with the Applied Biosystems™ QuantStudio™ 6 Flex Real-Time PCR System. The ΔΔCt method was used to calculate the mean fold changes representing the expression level of a gene in each sample; Livak K J, et al., Analysis of relative gene expression data using real-time quantitative PCR and the 2(-Delta C(T)) Method. METHODS (San Diego, Calif) 2001, 25(4): 402-408.

Statistical analysis. All clinical and biochemical results were summarized as means±SD and Median and Interquartile range (IQR) for skewed distribution. The inventors performed all statistical comparisons between the control and study group using T-test, Chi-square test, and Wilcoxon rank-sum test to calculate the P-value. A P-value<0.05 was considered statistically significant. All analyses were performed with the use of SAS software, version 9.4 (SAS Institute Inc., Cary, NC, USA).

Results/Demographic and clinical data. Table3 below displays the demographics and clinical and laboratory characteristics of the heatstroke patients on admission precooling (T0) and 4±3 hours post-cooling (T1) and heat-stressed subjects (control group). The ambient temperature and humidity in Makkah during the 5-days (30 Aug. to 3 Sep. 2017) pilgrimage ranged from 37 to 43° C. with a relative humidity of 35 to 49%, respectively. Seven patients had rectal temperature>42° C. (last three bars in FIG. 1), and eight presented in deep coma (GCS 3). Heatstroke patients exhibited rhabdomyolysis, renal dysfunction, and liver enzyme alteration. All patients were cooled by the same cooling method based on conduction and evaporation. After cooling was completed, five patients progressed to multi organ failure, including coma (GCS<6), a requirement of mechanical ventilation and vasopressor therapy to maintain their blood pressure. No patient died after a 28-days follow-up.

TABLE 3
Clinical characteristics of heatstroke patients pre-and post-cooling and control group*

| | Control | T0 | T1 | P-value | | |
|---|---|---|---|---|---|---|
| Parameters | (N = 19) | (N = 19) | (N = 17) | (Control vs. T0) | (Control VS. T1) | (T0 vs. T1) |
| Age (Gautier L, | 50 | 58 | 62 | 0.050\hat{ } | 0.011\hat{ } | NA |
| et al., affy- | (41, 55) | (45, 66) | (50, 66) | | | |
| analysis of | | | | | | |
| Affymetrix | | | | | | |
| GeneChip data | | | | | | |
| at the probe | | | | | | |
| level. | | | | | | |
| BIOINFORMATICS | | | | | | |
| 2004, 20(3): 307- | | | | | | |
| 315.sub) | | | | | | |
| Gender F/M | 8/11 | 11/8 | 9/8) | 0.330\hat{ }\hat{ } | 0.515\hat{ }\hat{ } | NA |
| Ethnicity n | | | | | | |
| North Africa | 8 | 3 | | | | |
| West Africa | 0 | 4 | | | | |
| East Africa | 3 | 1 | | | | |
| South Asia | 4 | 7 | | | | |
| West Asia | 3 | 4 | | | | |
| Core temp (° C.) | 36.3 | 41.7 | 38.1 | <0.0001* | 0.0008\hat{ } | <0.0001^{$$} |
| | (0.8) | (0.8) | (0.7) | | | |
| ALB (g/L) | 40 | 40 | 34 | 0.45\hat{ } | <0.0001\hat{ } | <0.0001^{$$} |
| | (39, 42) | (39, 42) | (32, 36) | | | |
| ALT (U/L) | 15 | 28 | 29 | 0.03\hat{ } | 0.006\hat{ } | 0.21^{$\hat{ }} |
| | (13, 26) | (21, 44) | (24.5, 46) | | | |
| AST (U/L) | 20 | 40 | 56 | 0.001\hat{ } | 0.01\hat{ } | 0.471^{$\hat{ }} |
| | (18, 24) | (25, 67) | (25, 71) | | | |
| BILI (umol/L) | 13.1 | 13.7 | 9.9 | 0.37\hat{ } | 0.67\hat{ } | 0.03^{$\hat{ }} |
| | (7.4, 14.7) | (8.6, 17.4) | (8.6, 12.1) | | | |
| CK (U/L) | 208 | 326 | 381 | 0.11\hat{ } | 0.02\hat{ } | 0.01^{$\hat{ }} |
| | (135, 291) | (135, 874) | (260, 1640) | | | |
| CREA (umol/L), | 73 | 133 | 130 | 0.0002* | 0.07\hat{ } | 0.018^{$$} |
| Mean (SD) | (13.44) | (55.99) | (66, 163) | | | |
| CRP (mg/L) | 8 | 5 | 4 | 0.50\hat{ } | 0.25\hat{ } | 0.77^{$\hat{ }} |
| | (4, 15) | (4, 12) | (3, 9) | | | |
| LDH (U/L) | 228 | 337 | 265 | 0.0004\hat{ } | 0.35\hat{ } | 0.14^{$$} |
| | (203, 268) | (272, 390) | (172, 348) | | | |

*Data are presented as Median (Q1, Q3) unless indicated otherwise.
**T -test/\hat{ }Wilcoxon rank sum test is used to calculate the P-value.
\hat{ }\hat{ }Chi-square test/**Fisher Exact is used to calculate the P-value.
^{$$}Paired T-test/^{$\hat{ }}Wilcoxon signed-rank test is used to calculate the P-value.
NA: Not applicable as there are no changes between pre and post cooling.

Gene expression signature of heatstroke. The analysis identified 8854 and 8723 genes that were differentially expressed (FC>log2 1.3, FDR<0.004) at T0 and T1, respectively, as compared with the control group (FIG. 2A). Downregulated gene proportion was higher at both 0 (n=5404; 61%) and T1 (n=5000; 57%), and 4911 differentially expressed genes were common to both time points (FIG. 2B).

Hierarchical clustering and principal component analysis (PCA) was used to visualize how the gene expression profiles of individual samples compare relative to each other (FIGS. 2C and 2D).

The unsupervised PCA (FIG. 2D) separated patients with acute heatstroke at T0 and T1 on principal component 2, driven by heat shock proteins (HSPs) encoding genes.

In addition, PC1 enriched in genes encoding proteins involved in bioenergetics and protein translation and ubiquitination distinguished two clusters of heat-stressed subjects, suggesting distinct or different stages of the heat stress response.

Volcano plots of DE genes in heatstroke patients T0 and T1 versus heat-stressed controls showed that at T0, HSP, cochaperones, and chaperonins genes are the most significantly expressed genes with the highest fold-changes, consistent with a reprogrammation of the transcriptome toward the stress response (FIGS. 2E and 2F)

Heat shock proteins. The genes whose expression increased the most were those involved in protecting the proteome from misfolding and aggregation. One hundred and fifty nine (48.1%) DE genes out of the 330 members of the human chaperone, which comprises the ensemble of all cellular molecular chaperone and cochaperone proteins, were found indicating a broad HSR. It was noted that these HSPs function in most compartments of the cells, including the cytoplasm, nucleus, mitochondria, and ER (FIGS. 3A and 3B).

The highest expressed inducible HSPs genes were HSPA1A, HSPA1B, HSPA6, HSPA4L, HSPB1, DNAJA4, DNAJB1, DNAJB4, and/or FKBP4 (FKBP Prolyl Isomerase) which exhibited 7 to 49 times fold changes relative to heat-stressed controls indicating a robust HSR (FIG. 3C). In addition to inducible HSP genes upregulation, a marked increase of cochaperones and chaperonins genes, such as BAG2, HSP60, and TRiC (T-complex protein Ring Complex) were detected.

Heat shock factors. Heat shock factors. The expression of heat shock genes is regulated at the transcriptional level by activating the heat shock transcription factors (HSFs); Gomez-Pastor R, et al., Regulation of heat shock transcription factors and their roles in physiology and disease. NATURE REVIEWS MOLECULAR CELL BIOLOGY 2018, 19(1):4-19. Using the upstream regulator analysis tool of IPA, 187 transcription factors (TF) were identified, including HSF1 and HSF2, that may explain the differentially expressed genes in heatstroke patients at T0. The inventors further disclose other chaperones and other molecules which can be used to evaluate heatstroke and heat-related conditions, see the supplementary tables below.

Supplementary Table 2A describes a list of chaperones in significantly expressed genes in heatstroke at T0.

Supplementary Table 2B describes a list of chaperones in significantly expressed genes in heatstroke at T1.

Supplementary Table 3A describes a list of all upstream regulators identified at T0 using a threshold of logP value=1.301 (p<0.05).

Supplementary Table 3B describes a list of all upstream regulators identified at T1 using a threshold of -logP value=1.301 (p<0.05). In some embodiments of the methods disclosed herein one or target molecules such as mRNA described in these supplementary tables may be detected in addition to the nine target molecules disclosed herein.

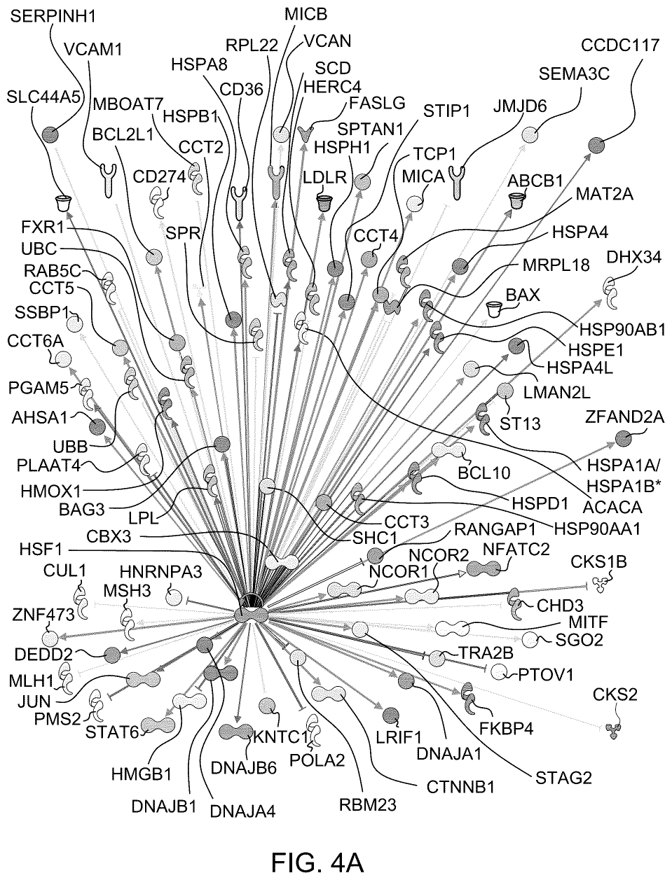

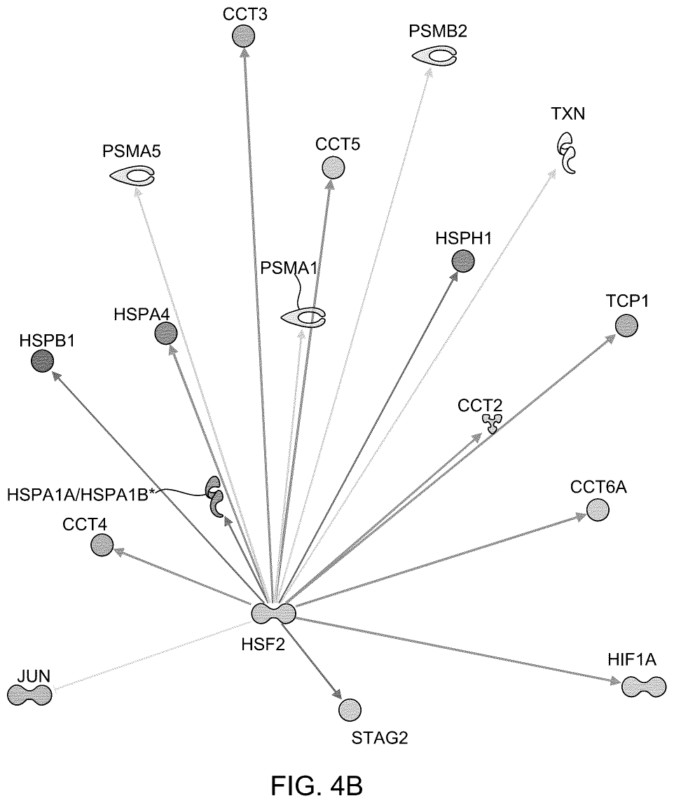

The top three TF included HNF4A (hepatocyte nuclear factor 4 alpha). TP53 (tumor protein p53), and MYC (MYC proto-oncogene, BHlh), which accounted for close to 2000 DE genes in the experimental dataset. HSF1 and HSF2, known regulators of the HSR, including its major component, the HSP gene expression, were significantly associated with 91 and 17 DEG in our patients based on the p-value of overlap (p=1.70E-06 and p=6.83E-03, respectively (FIGS. 4A and 4B).

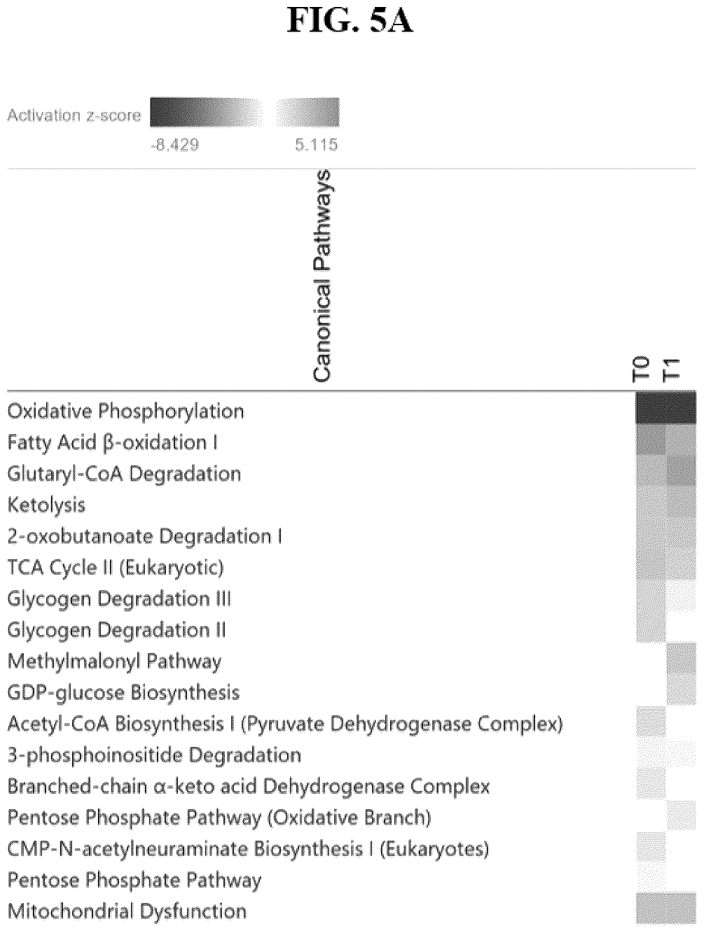

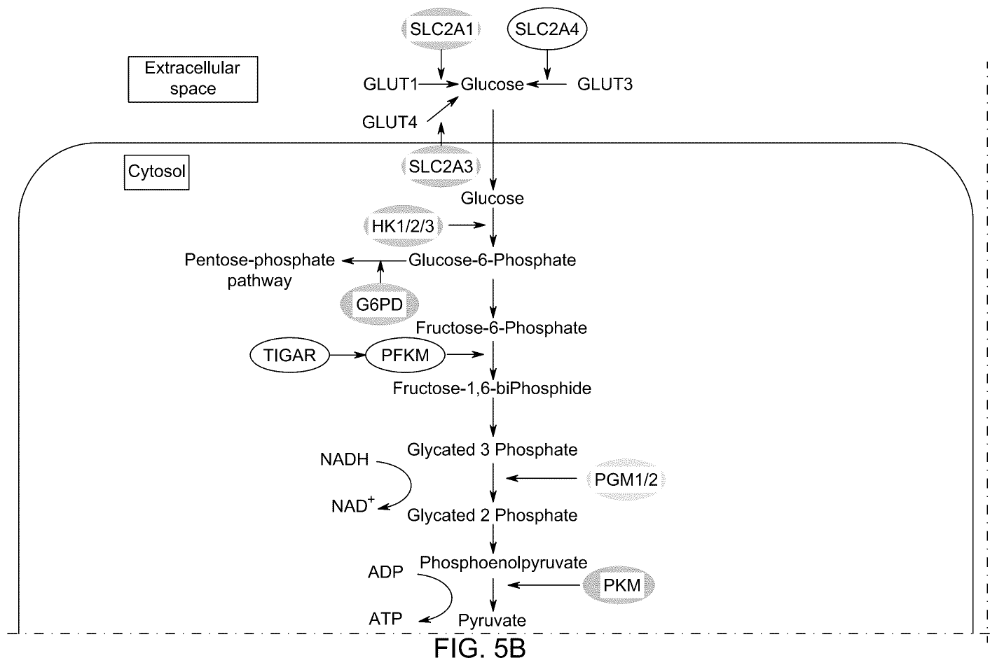

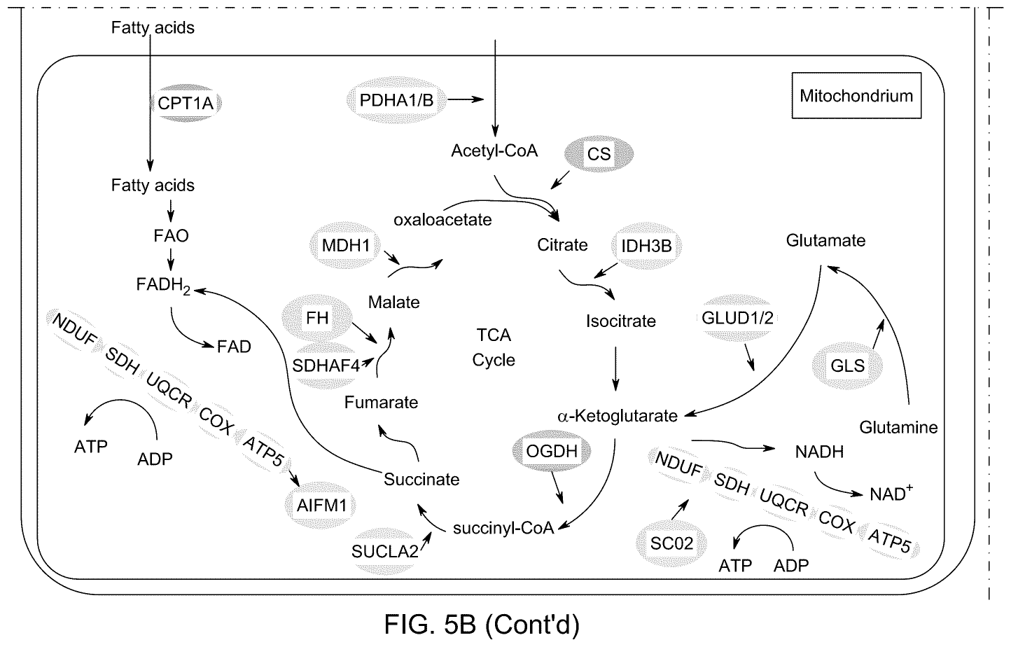

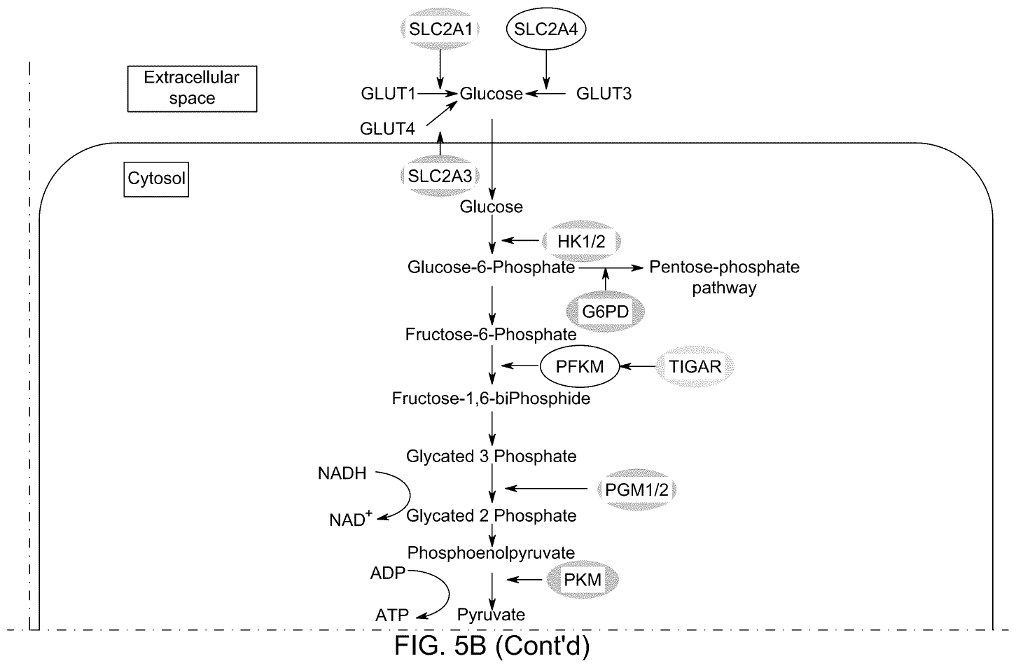

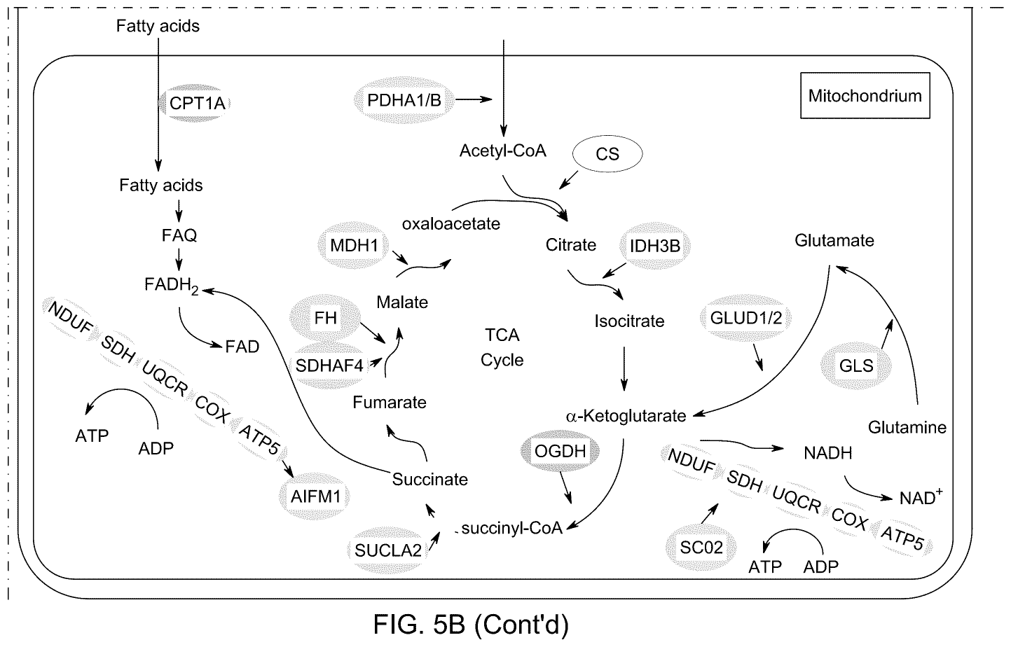

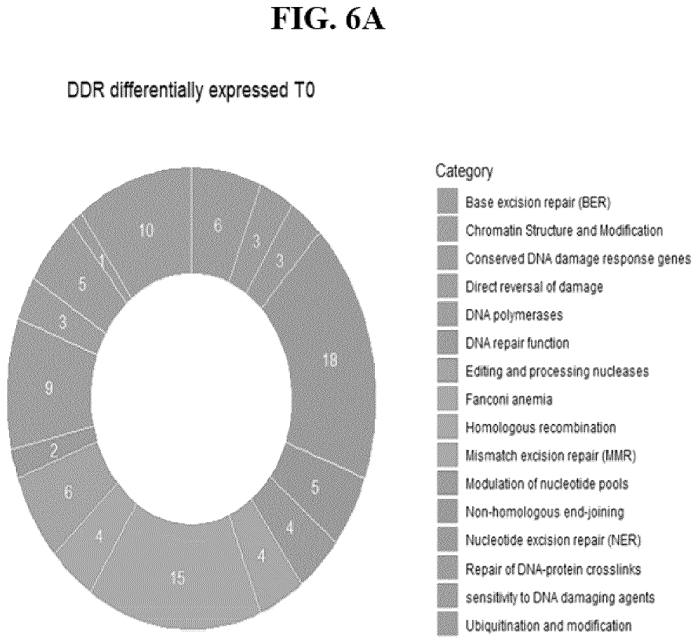

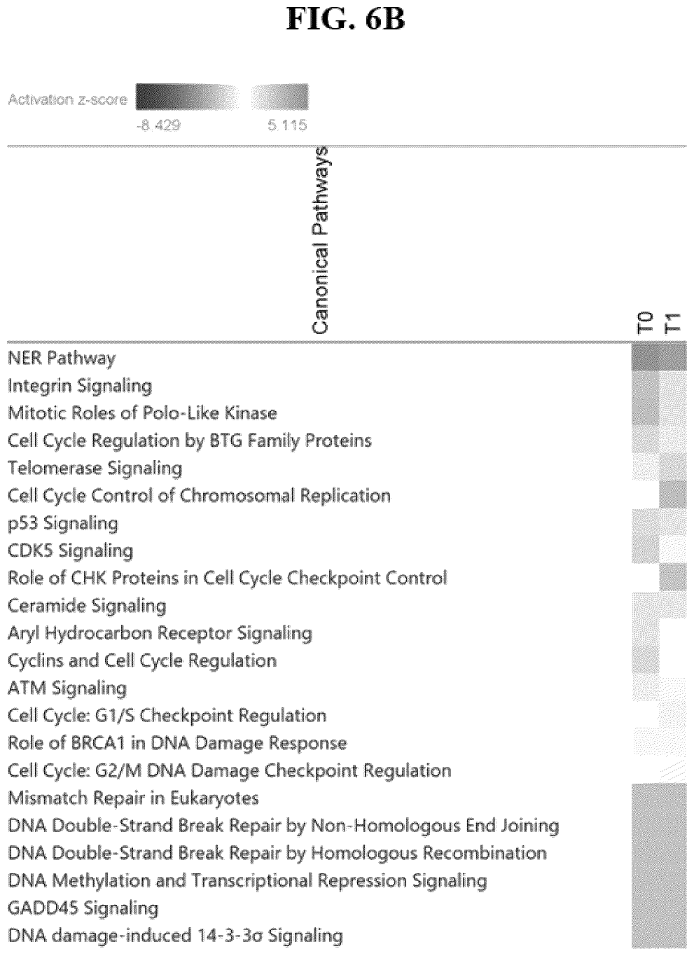

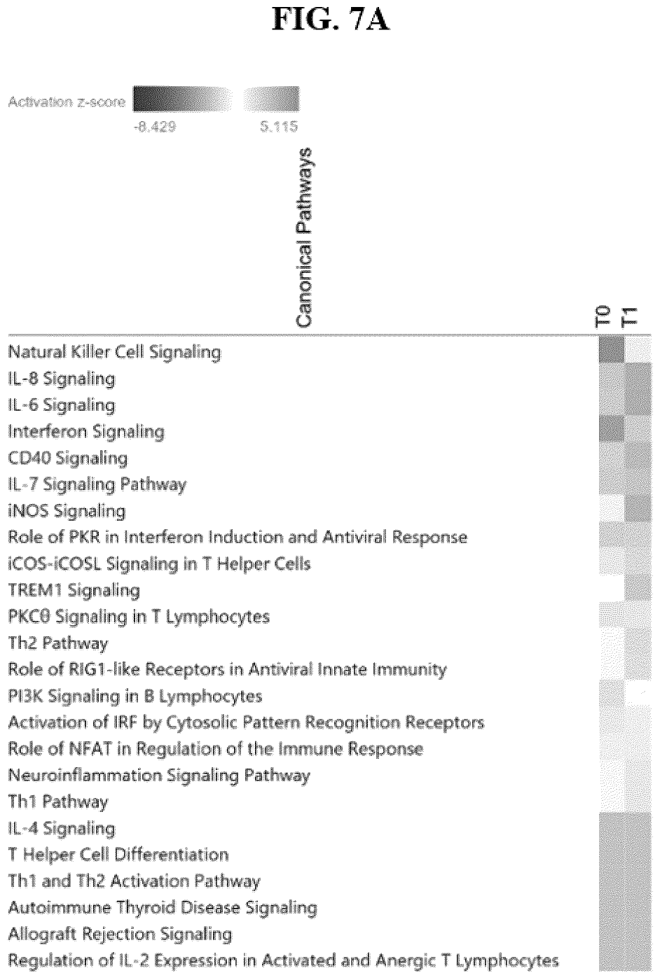

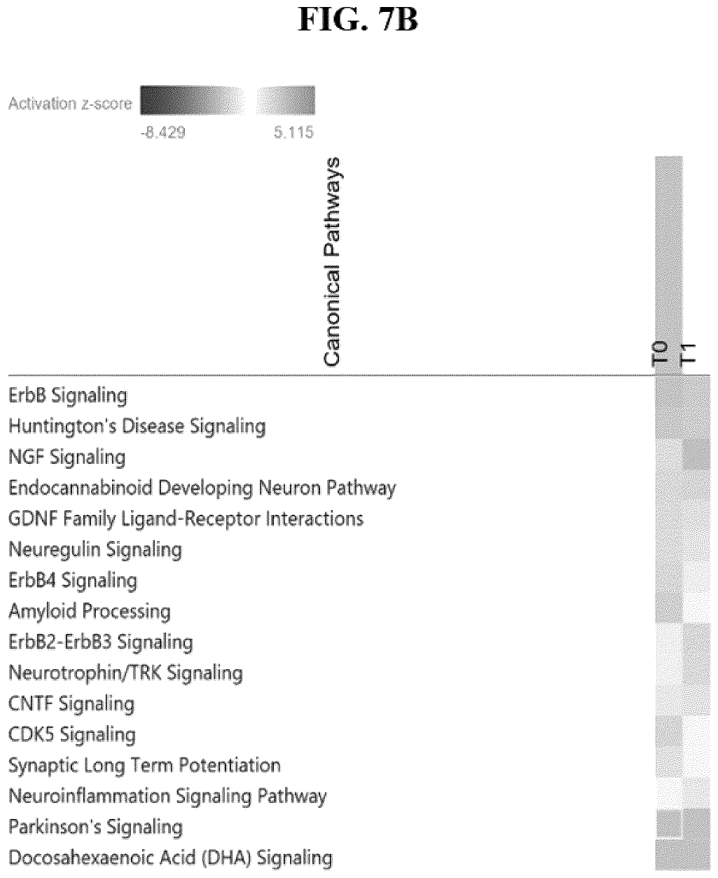

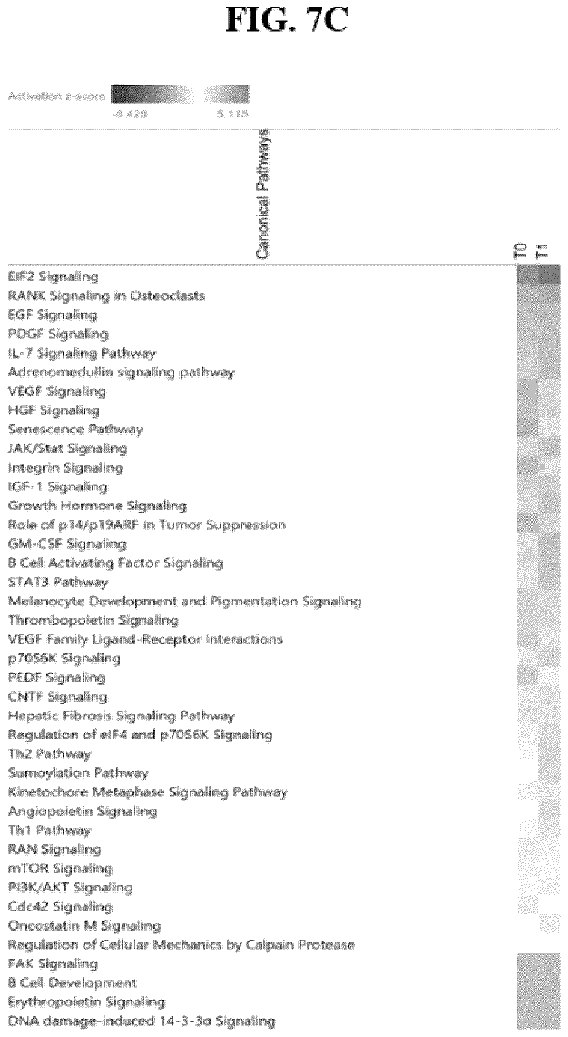

Metabolic and signaling pathways of heatstroke. The analysis identified the changes in metabolic and signaling pathways and biological processes underlying the complex pathophysiology of heatstroke on admission and during recovery. The most significantly enriched pathways were related to proteostasis (FIG. 3D), bioenergetics and oxidative stress (FIG. 5A), DNA damage response (DDR) (FIGS. 6A-6B), immune response, CNS signaling, and cellular growth, proliferation, and development (FIGS. 7A-7C).

Alteration of proteostasis. Seven significantly enriched pathways related to the regulation of proteostasis (FIG. 3D) were identified; Balch W E, et al., Adapting proteostasis for disease intervention. SCIENCE (New York; NY) 2008, 319(5865):916-919. These include protein synthesis (EIF2, regulation of eiF4 and P70S6K signaling), the folding and conformational maintenance (UPR pathway), and protein degradation (ubiquitin-proteasome, BAG2, and autophagy signaling pathways).

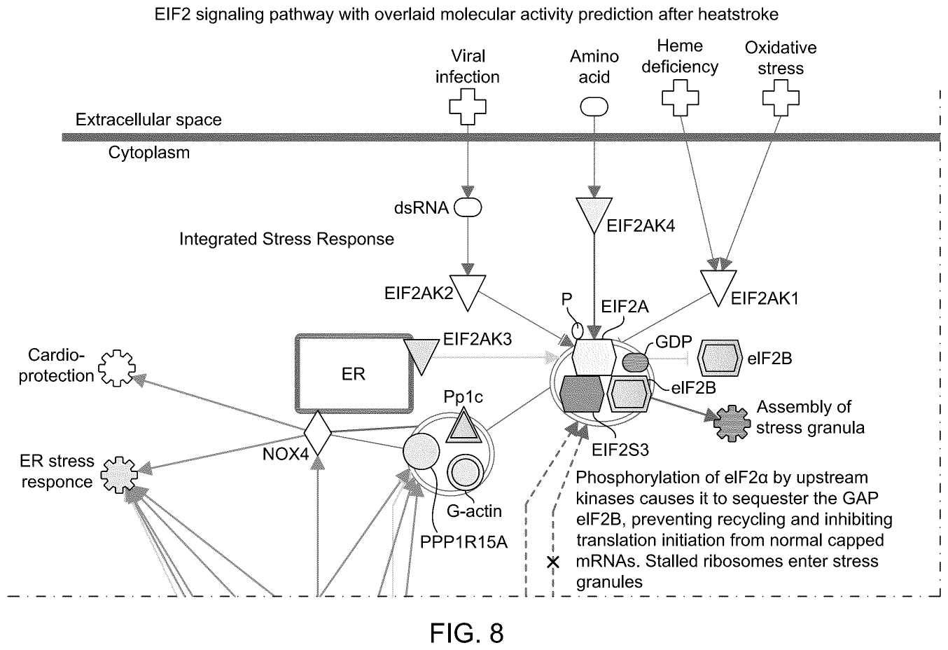

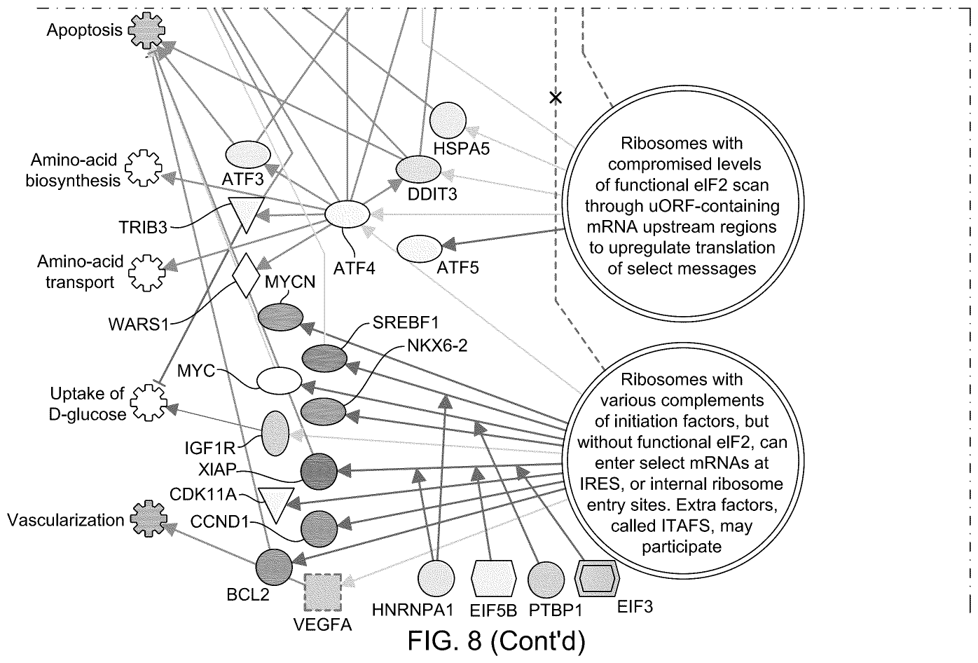

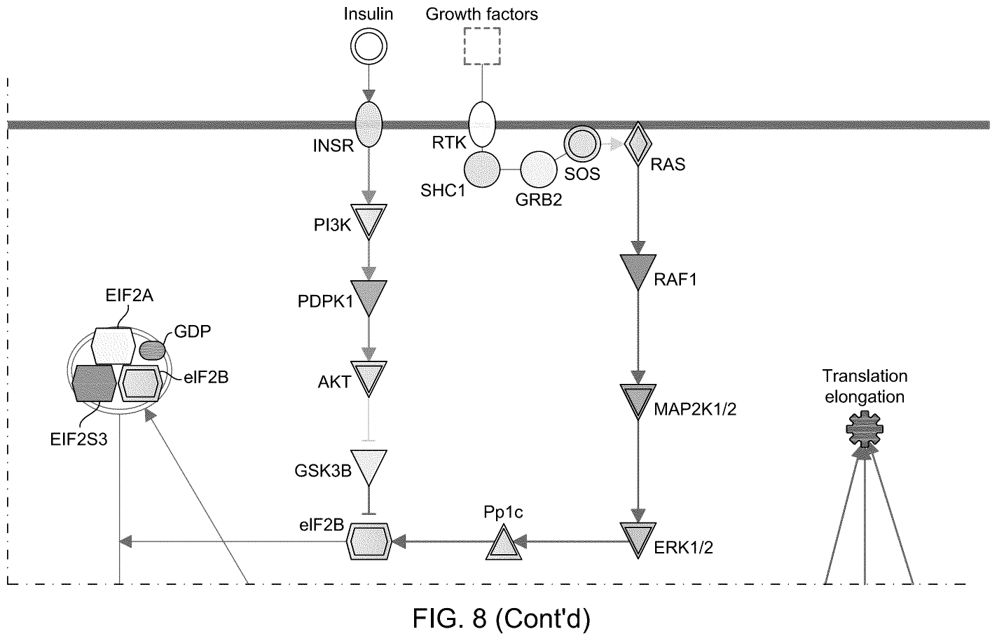

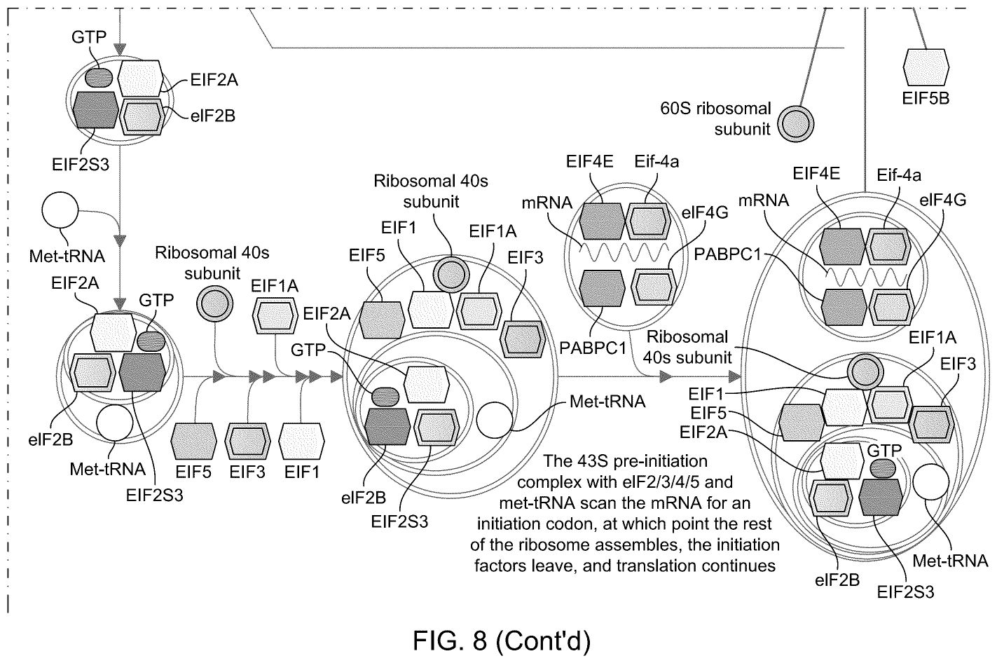

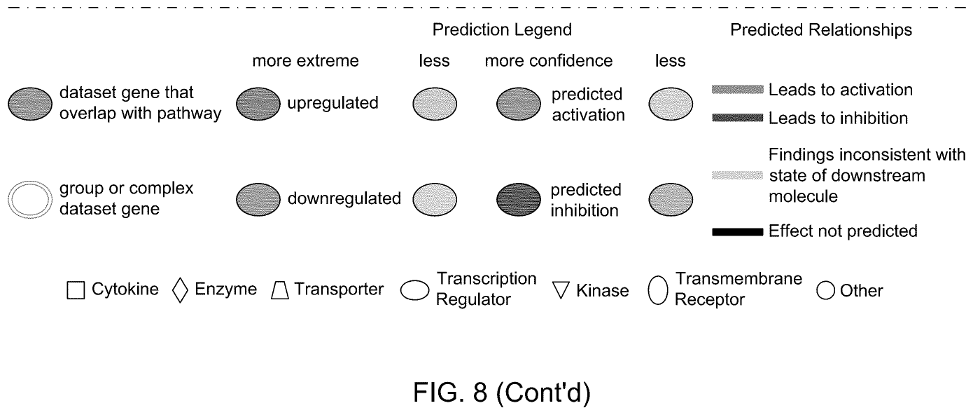

Integrated stress response. Upon stress, eukaryotic translation initiation factor-alpha (Eif2α) is rapidly phosphotylated, triggering a signaling cascade that leads to the arrest of the initiation of protein synthesis at the ribosomal level (called integrated stress response or ISR); Pakos-Zebrucka K, et al., The integrated stress response. EMBO REPORTS 2016, 17(10)1374-13951PA analysis predicted that upregulated mitogen-activated protein kinase (MAPK) gene through complex signaling pathway results in inhibition of protein translation elongation and activation of ER stress response and apoptosis (FIG. 8).

The p38 MAPK plays a central role in the early transcriptional response to many stressors, including environmental heat. Four kinases were identified to activate EIF2α phosphorylation, including PERK (also known as PKR-like ER kinase), which is encoded by EIF2AK3 (eukaryotic translation initiation factor 2 alpha kinase3) gene; were significantly upregulated in heatstroke patients at T0, indicating that PERK may be the kinase that triggers ISR in heatstroke (FIG. 8).

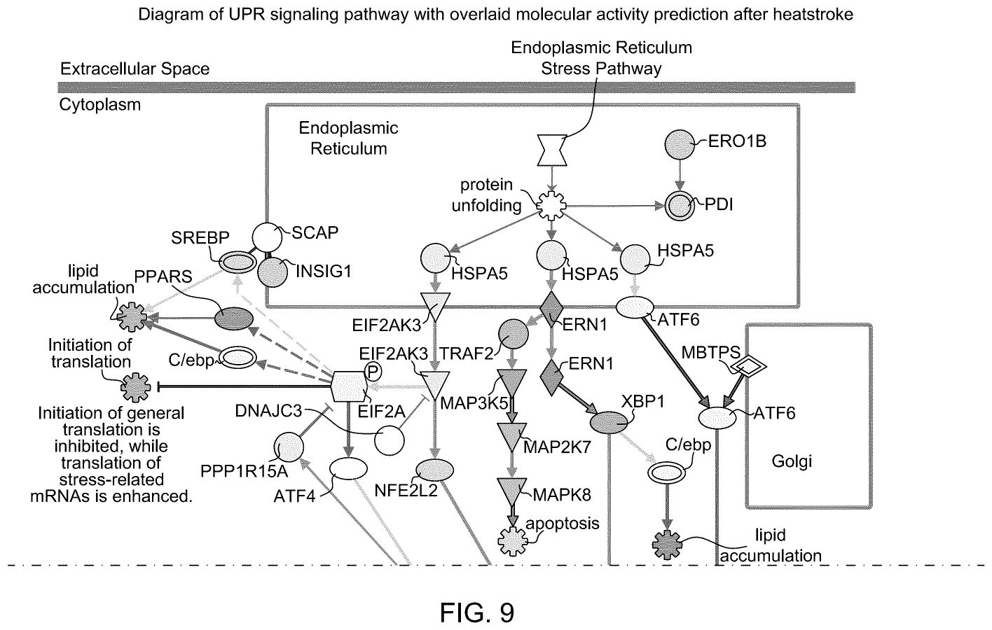

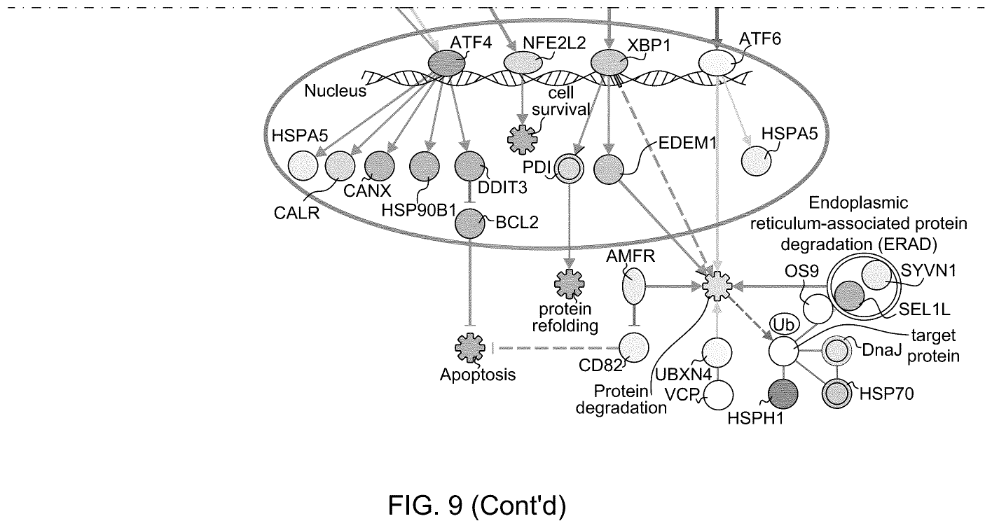

Unfolded protein response. The unfolded protein response (UPR) was actuated when sensor proteins detect excessive accumulation of misfolded proteins in the ER and mitochondris. The inventors identified the upregulation of IRE1 and PERK genes that encode for transmembrane protein sensors, which detect misfolded proteins in ER, while the third known sensor, ATF6, was downregulated (FIG. 9). IRE1, through activation of XBP1, export and degrade misfolded proteins, while PERK reduces protein synthesis via the phosphorylation of EIF2. Likewise, HPA5 was upregulated; this gene encodes a specific chaperone to the ER, which plays a crucial role in protein folding and quality control. The UPR in mitochondria was indicated by the increased expression of several genes that promote mitochondrial protein homeostasis, including HSPD1, HSPE1, and HSPA9.

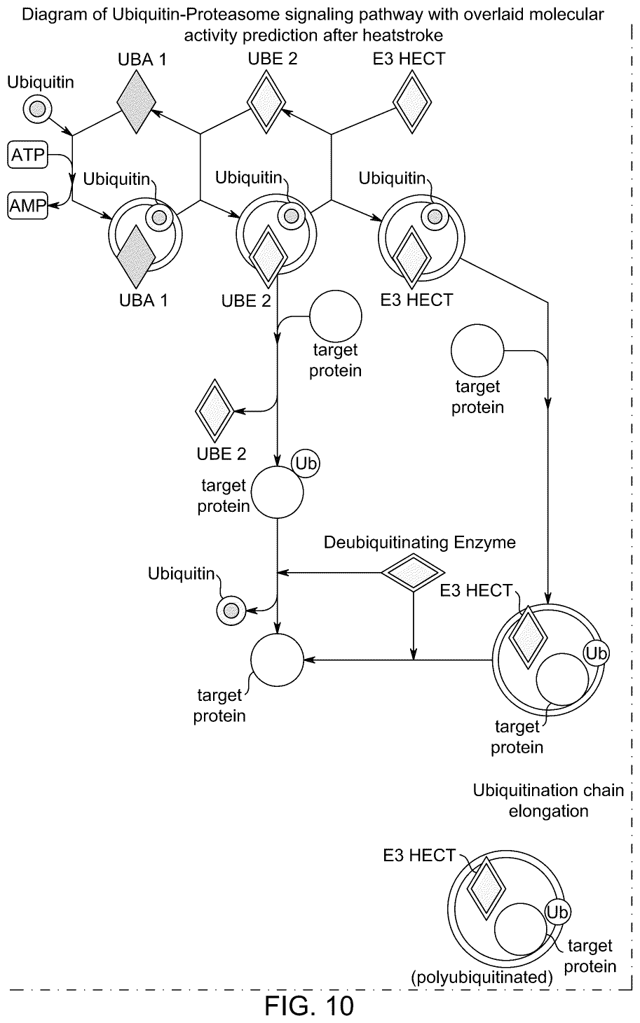

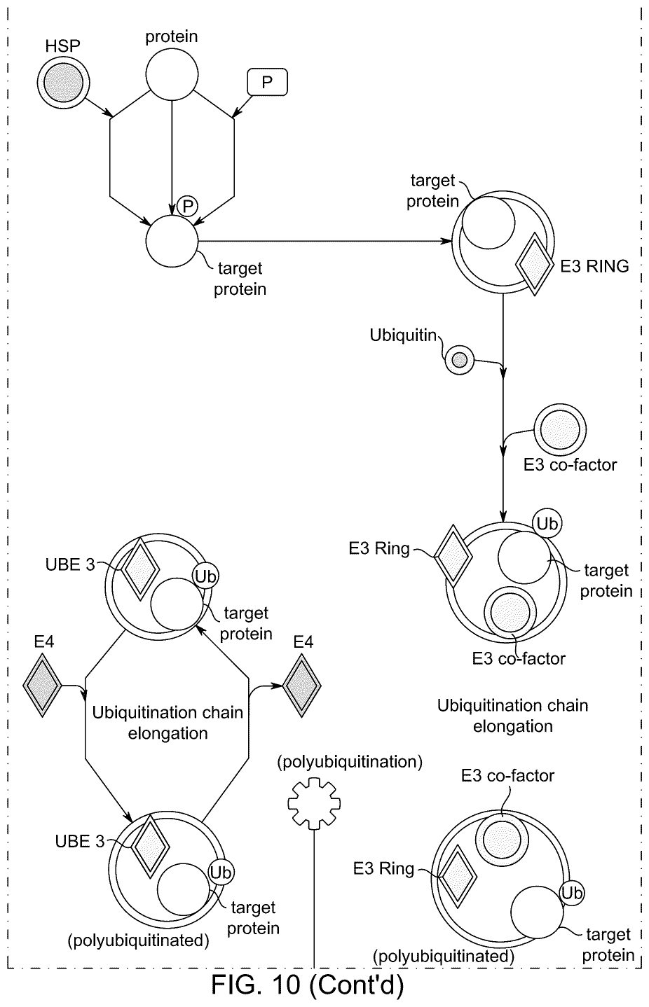

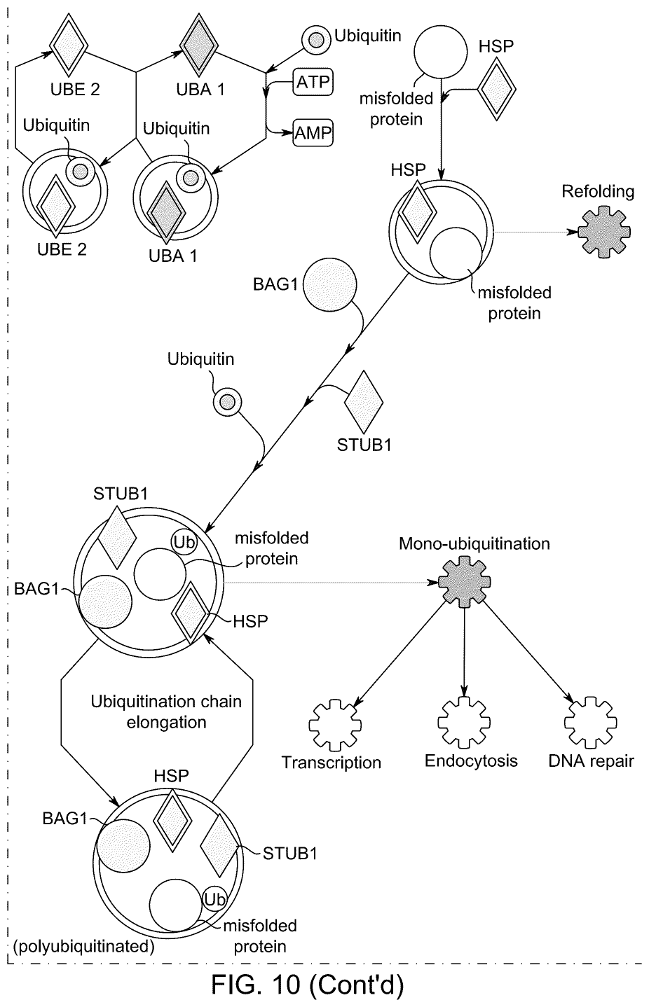

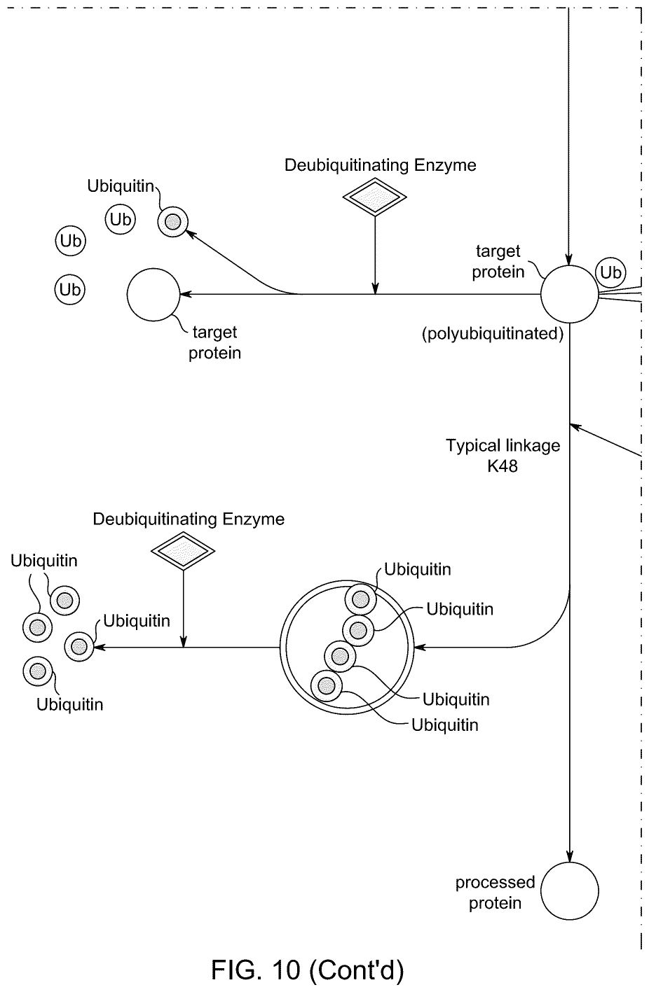

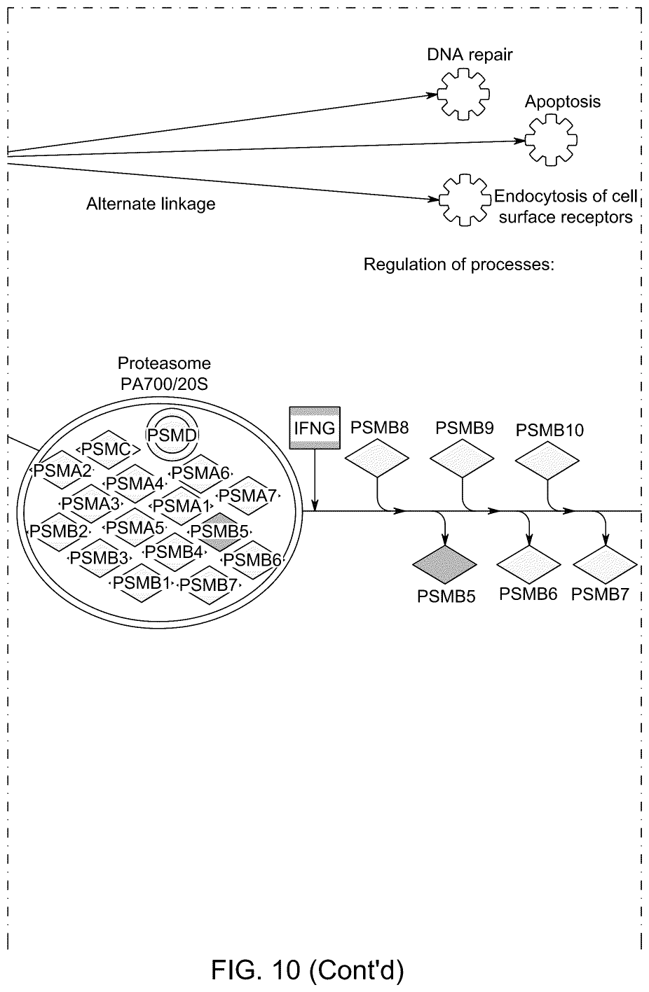

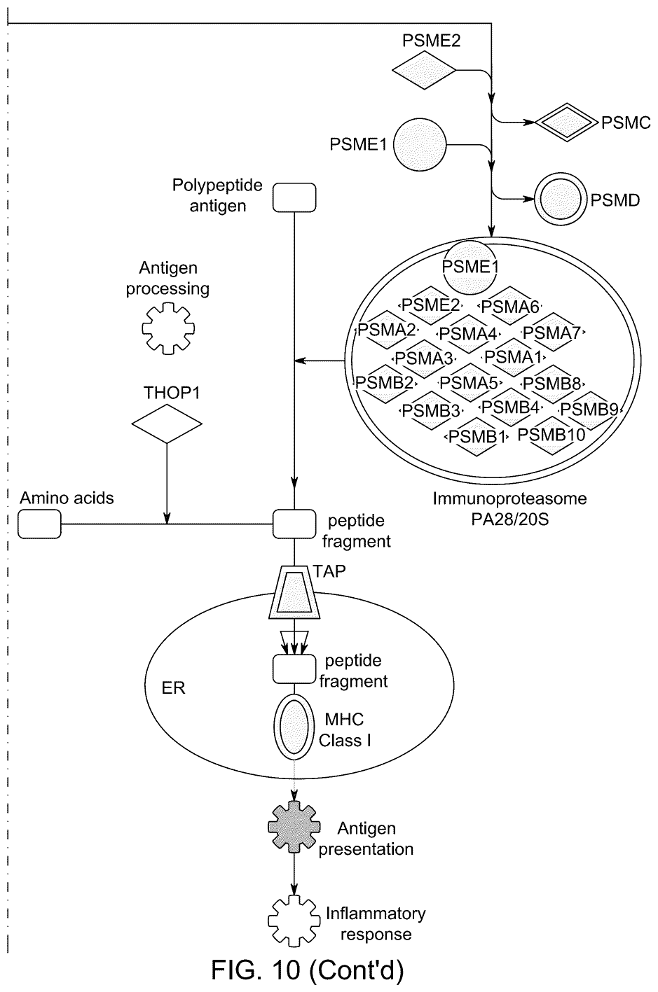

Ubiquitin-proteasome pathway. Misfolded and aggregated proteins that cannot be corrected are eliminated by degradation through the proteasome ubiquitination pathway and autophagy; Walter P., et al., 2011 supra. The inventor found that UPP but not the autophagy pathway was significantly enriched at both time points in heatstroke patients (FIG. 10). However, the present data showed a sustained a) decreased expression of roost of the genes E1 (ubiquitin-activating), E2 (ubiquitin-conjugating), and E3 (ubiquitin ligating) that tag the target proteins for elimination; as well as b) all the proteasome endopeptidase genes including those of the immunoproteasome, which cleave the peptide bonds consistent with a reduction of protein degradation following heatstroke. Overall, the significant expression of genes related to the HSR, ISR, UPR, and proteasome ubiquitination simultaneously indicates that heatstroke elicits a major alteration of the proteostasis network.

Hypoxia and oxidative stress. The gene expression data unveiled an upregulation of Hypoxia Inducible Factor 1 Subunit Alpha (HIF-1A) and Nuclear Factor, Erythroid 2 Like 2 (NFE2L2) genes at T0 and T1. HIF-1A is a master transcriptional regulator of the adaptive response to hypoxia. NFE2L2 encodes a transcription factor that regulates genes that contain antioxidant response elements (ARE) in their promoters. These include SOD1 (superoxide dismutase), GSTM2 (Glutathione S-Transferase Mu 2), and HMOX1 (Heme oxygenase), which were significantly increased in the present dataset, indicating that hypoxia and oxidative stress occurred in heatstroke patients.

Energy metabolism. Heatstroke patients displayed marked alteration of genes involved in energy production, including oxidative phosphorylation (OxPhos), ATP production, glucose metabolism, as well as alternative pathways such as beta-oxidation of fatty acid and glutamine metabolism (FIGS. 5A and 5B).

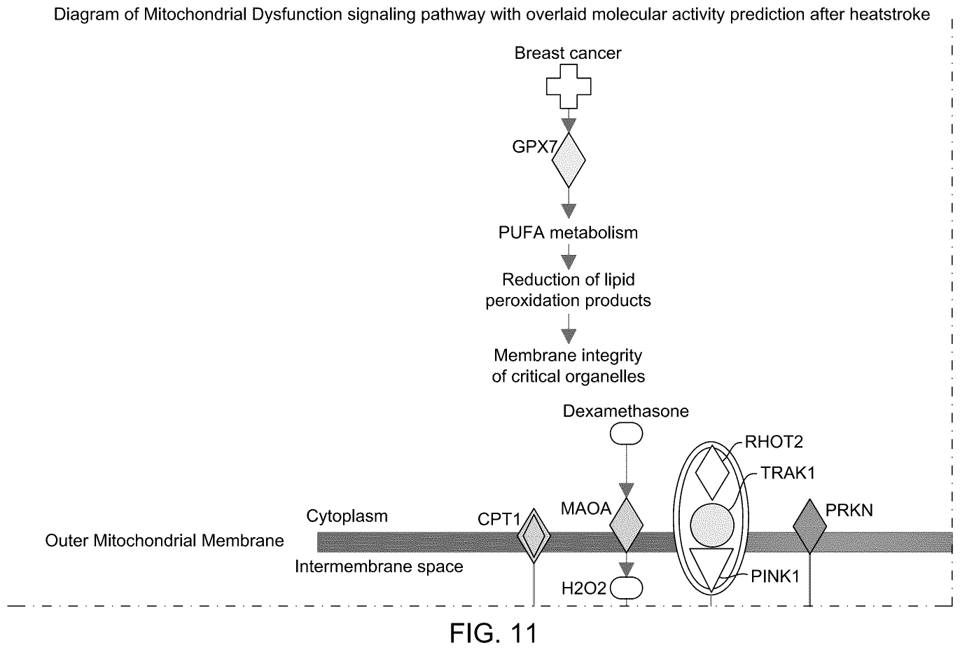

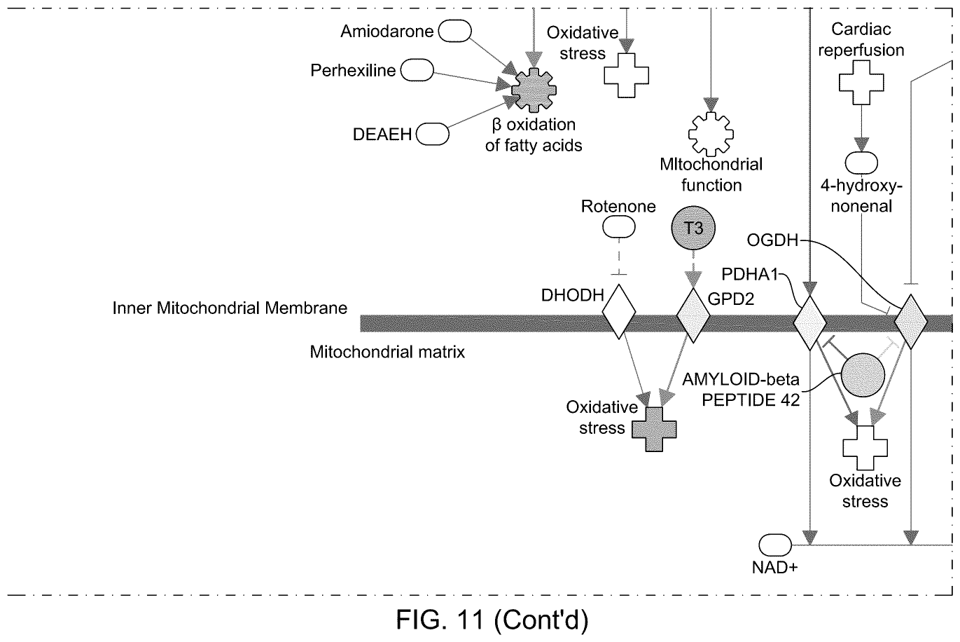

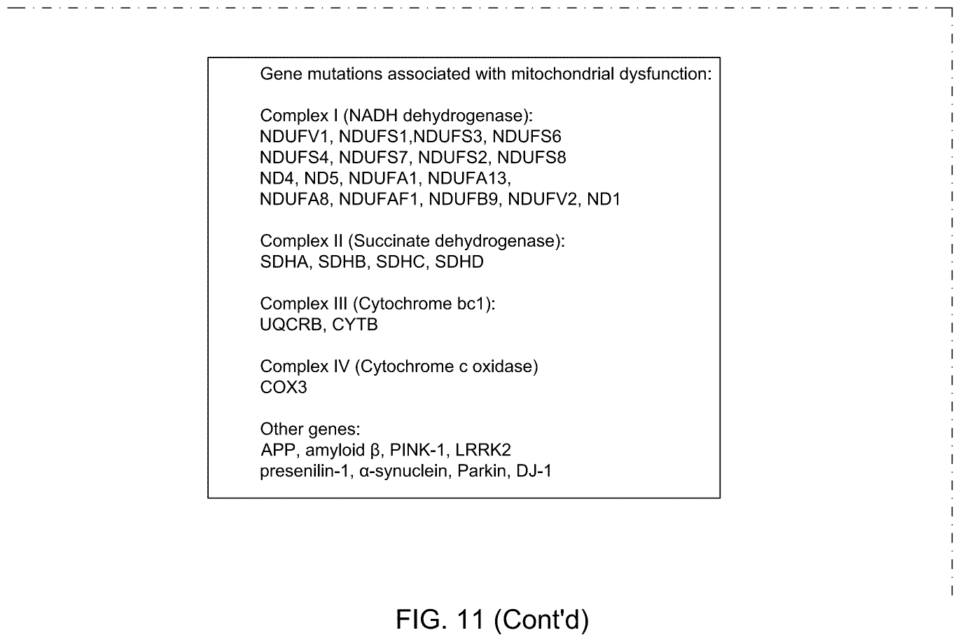

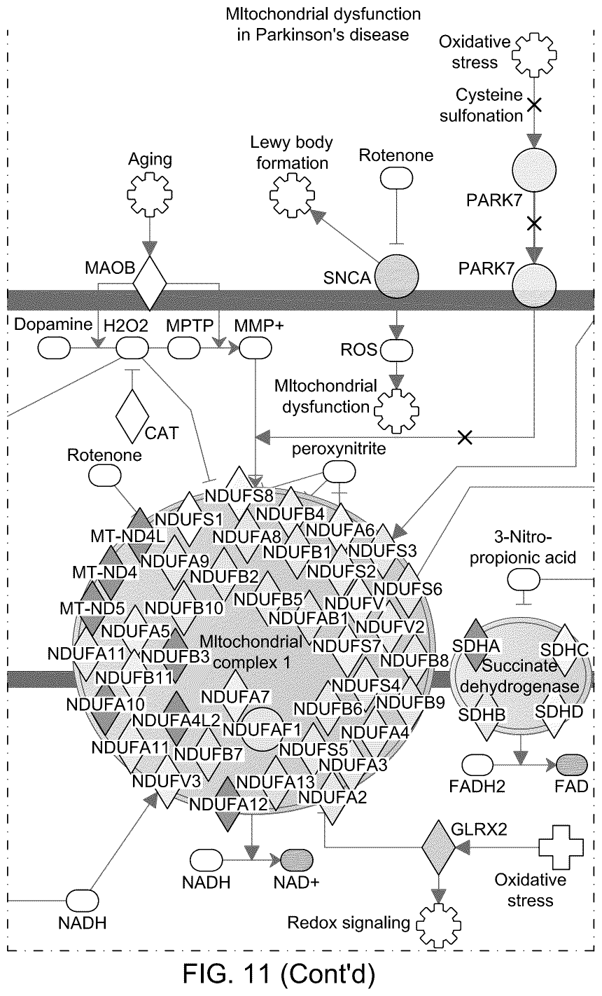

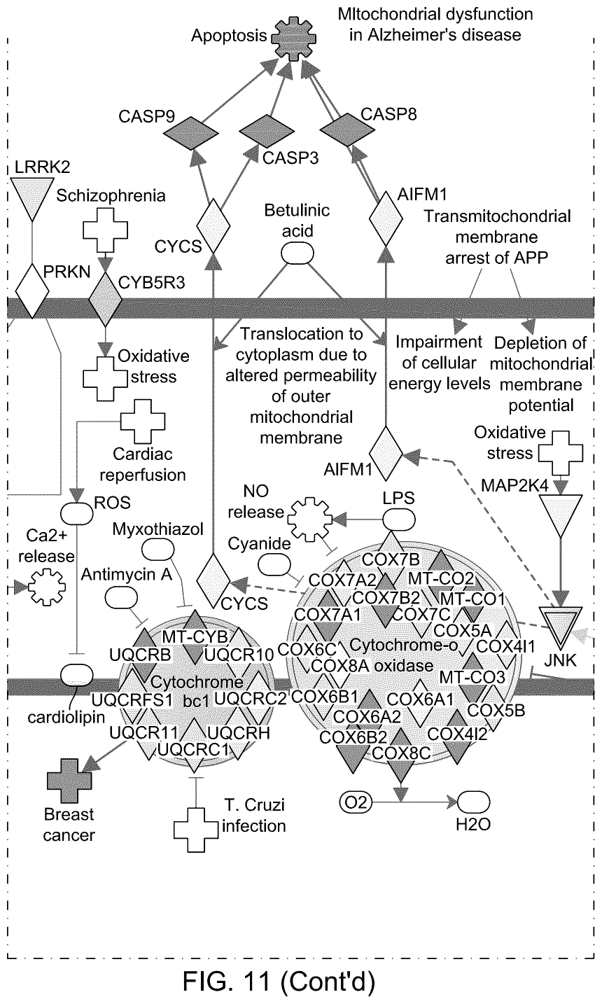

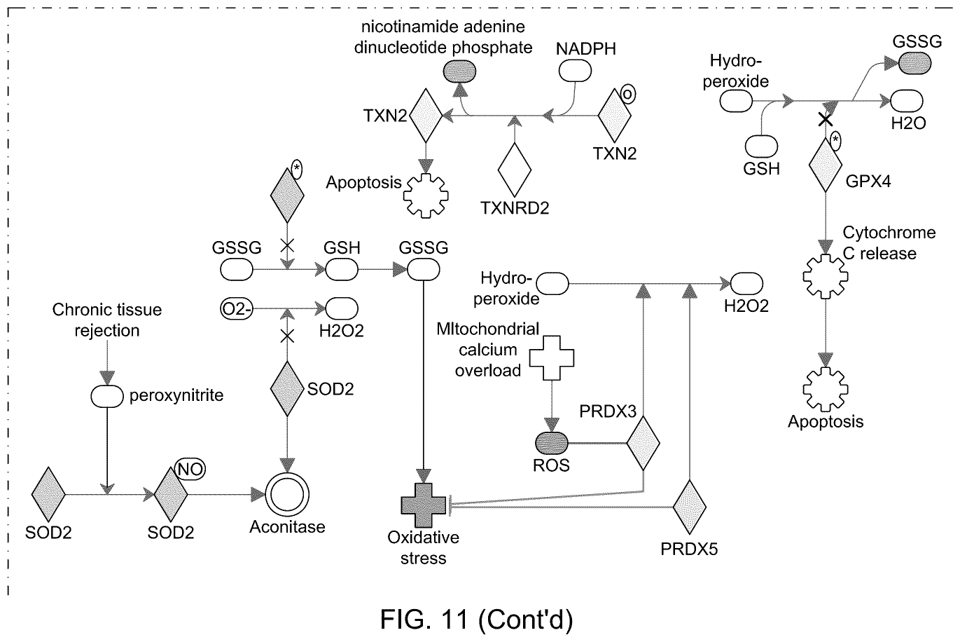

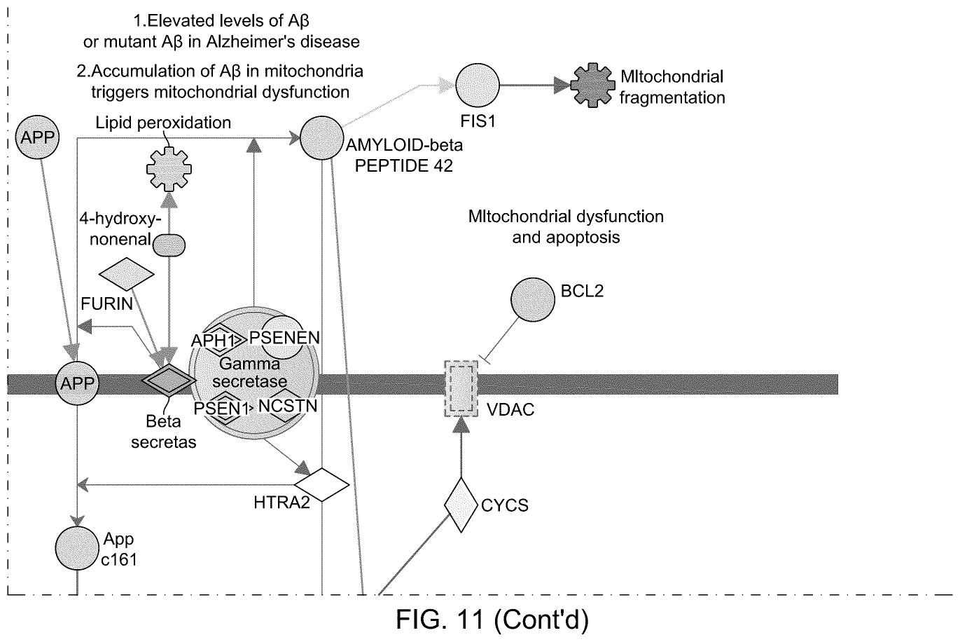

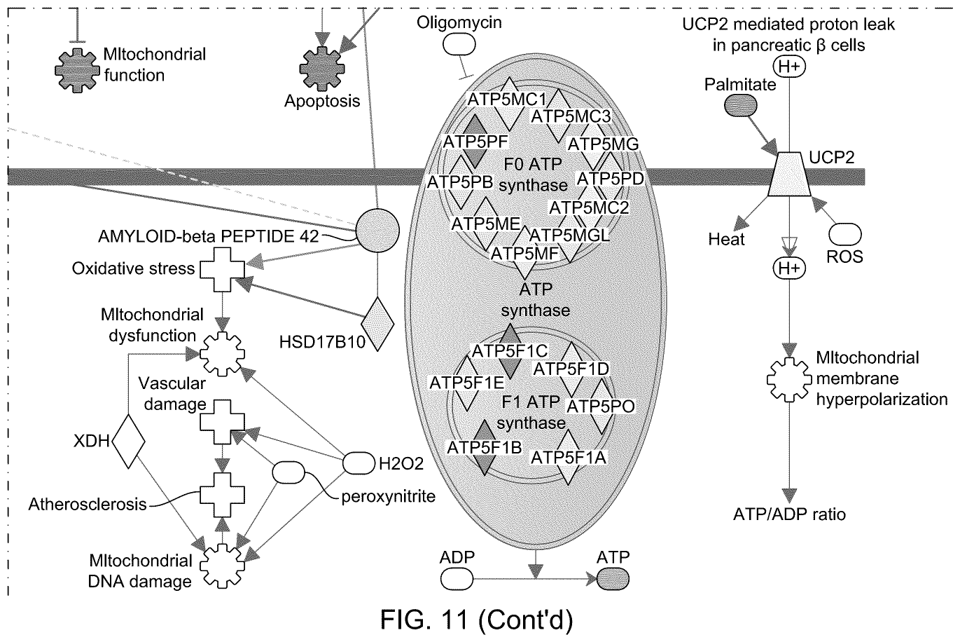

Oxidative phosphorylation and ATP production. Gene expression data showed that all the genes involved in mitochondrial electron transport chains, NDUF (complexes I), SDH (complex II), UQCR (complex II), COX (complex IV), and ATP synthase (complex V) were downregulated (FIG. 11). Also, the pyruvate dehydrogenase gene (PDHAA1) expression was decreased. This gene encodes a mitochondrial multiple enzyme complex that catalyzes the conversion of pyruvate to acetyl-CoA, hence inhibiting the main link between glycolysis and the tricarboxylic acid (TCA) cycle, Notably, a simultaneous decrease in the expression of the synthesis of cytochrome C oxidase (SCO2) and apoptosis-inducing factor 1 (AIFM1) was observed (FIG. 5B). These two genes are crucial for the transfer of electrons from the reduced form of nicotinamide adenine dinucleotide (NADH) and Flavin adenine dinucleotide (FADH) to oxygen molecules in the electron transport chain. They hence indicate altered energy production from fatty acid oxidation or glutamine metabolism. These results indicate that heatstroke induced mitochondrial dysfunction and inhibition of the conversion of glucose, amino-acid, and lipid into ATP.

Glucose metabolism. Gene expression data showed that genes encoding for transporters facilitate the cellular uptake of glucose, namely glucose transporter type 1 (SLC2A1) and type 3 (SLC2A3) but not type 4 (SLC2A4) were significantly upregulated. There was also increased expression of hexokinase (HK1), which phosphorylates glucose to produce glucose-6-phosphate, the first step in glucose metabolism together with Pyruvate kinase (PKM). Pyruvate kinase accelerates the last step of the glycolysis by dephosphorylation of phosphoenolpyruvate to pyruvate, leading to ATP production. In contrast, Phosphoglucomutase (PGM2), which facilitates the interconversion of glucose 1-phosphate and glucose 6-phosphate, decreases, slowing the downstream oxidation of glucose to pyruvate. Consequently, the findings suggest a disturbed glucose metabolism, although the net result in ATP production is difficult to ascertain. Overall, the analysis indicates that patients subjected to extreme heat may fail to maintain energy to sustain the stress response, and this could have contributed to the progression to heatstroke.

DNA Damage response. The gene expression data revealed several differentially expressed genes for proteins involved in DNA repair and maintenance (FIGS. 6A and 6B), indicating that the cells were mounting a response to preserve DNA integrity. These include the nucleotide excision repair (NER), the mismatch repair in eukaryotes (MMR), and both the homology-dependent recombination (HDR) and non-homologous end joining (NHEJ), which repair DNA base damage, DNA strand breaks, and complex events like interstrand crosslinks, respectively (FIG. 6A). Likewise, several genes were involved in the ataxia-telangiectasia mutated protein (ATM) pathway and signaling cascades that follow DNA strand breaks. These comprise the activation of checkpoints that control cell division, P53, BRCA, and GADD45, consistent with DNA damage and repair mechanisms. In addition, pathway analysis showed that many signaling pathways such as cell cycle arrest, apoptosis, and senescence were activated.

Activation of innate immunity. Several genes involved in innate immunity showed marked alteration in expression following heatstroke. TNF-α and IL-6, which are the key drivers of inflammation in heatstroke, were not detected at the transcriptional level in this cohort. Nevertheless, several TNF-related pathway genes and members of the IL-6 family were differentially expressed. Several acute phase response genes such as haptoglobin (HP), orosomucoid 1 (ORM1) and 2 (ORM2), bactericidal permeability-increasing protein (BPI), and triggering receptor expressed on myeloid cells 1 (TREM1) were all upregulated at T0 and increased further at T 1. Genes that mediate the recruitment and activation of neutrophils, macrophages, and monocytes were also upregulated, such as CD177, Matrix Metallopeptidase (MMP) 8 and 9, and Elastase, Neutrophil Expressed (ELANE) genes. Pathways analysis showed that DE expressed genes were concurrently involved in pro-inflammatory (IL-8, IL-6, IL-15, IL-17, IL-22, IL-23, and TREM1) and anti-inflammatory (IL9, IL4, and IL10) pathways (FIG. 7A).

The inflammation is mediated by the NFkB signaling pathway, although the overall phenotype at T0 and T1 tends to lean toward Th2. The innate immune response included several genes involved in dendritic cell maturation, antigen-presenting cells, and T-cell exhaustion. Most of these genes, including all MHC1 and 2, were downregulated, suggesting a decreased cellular immunity. Overall, these results add further evidence that heatstroke elicits a complex innate immune response mediated through NFKB signaling.

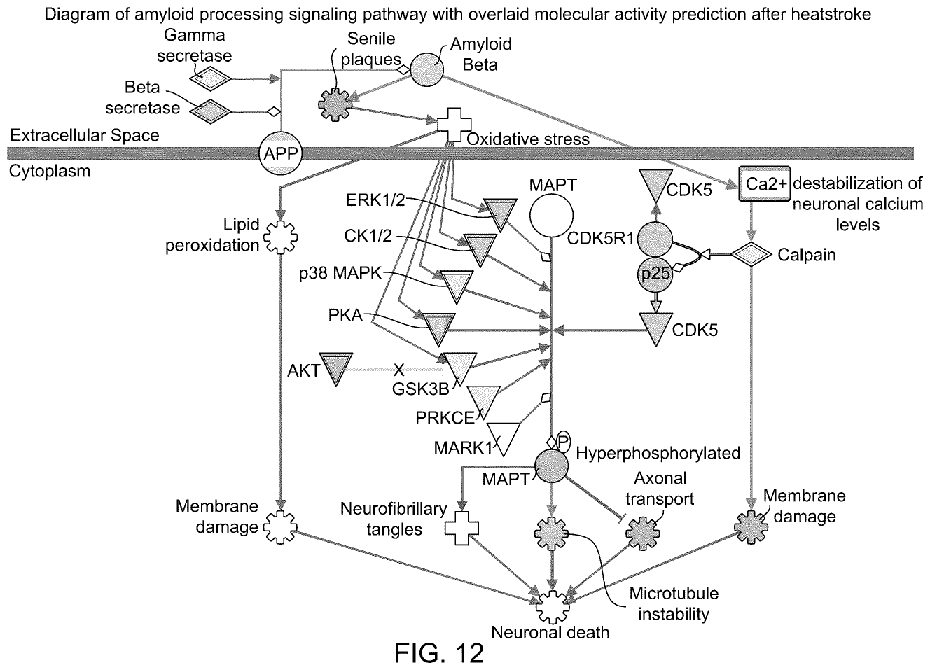

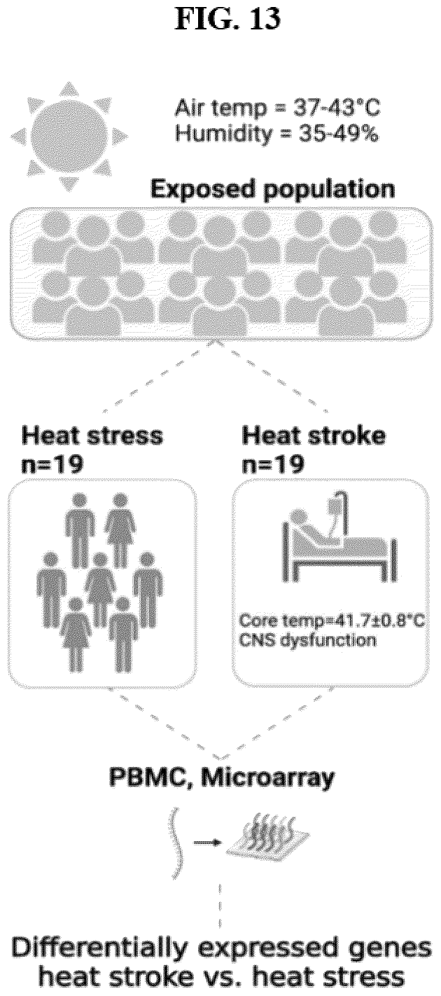

Central Nervous System signaling. The data showed increased APP (amyloid Beta precursor protein) gene expression and significant enrichment of the amyloid processing pathway predicting activation of senile plaque formation (FIG. 12). Likewise, neurodegenerative disease pathways, i.e., the Huntington's and Parkinson's disease signaling or linked to their pathogenesis such as neuregulin, ErbB, and Docosahexaenoic acid signaling pathways, were significantly associated with our DE genes (FIG. 7B).

Cellular growth, proliferation, and development. One of the universal responses to stress is the inhibition of growth and proliferation and directs the transcriptomic response to stress-related functions. Surprisingly, the findings of the present disclosure showed the opposite, as most of the metabolic and signaling pathways were activated (FIG. 7C).

Validation of Microarray Data. Twenty representative genes were selected from each time point to validate the microarray data by Qrt-PCR. The inventors found a good concordance between microarray and RT-qPCR data using Pearson correlation (P<0.05). Quantitative real-time PCR was performed for the 20 randomly selected genes on admission with heatstroke (T0) and after cooling therapy (T1). Fold-change represents the expression level of genes after heatstroke relative to baseline. Concordance between microarrays and RT-qPCR was determined by Pearson Correlation (p<0.05). See Supplementary Table 4 at the end of the specification.

As shown herein, the inventors for the first time have revealed a genome-wide transcriptional program of blood mononuclear cells in heatstroke patients and how it differs from that in individuals subjected to the same environmental heat but who do not develop this condition. This program is enriched in stress-related functions, including HSR, UPR, energy metabolism, DDR, immune response, and CNS signaling. Unexpectedly, the transcriptome included multiple metabolic and signaling pathways for growth and proliferation.

Gene expression analysis predicted several perturbations in key signaling pathways of this program, including inhibition of protein degradation machinery, failure to repress cellular growth and proliferation, and a reduction of energy production. Further, the findings revealed that cooling therapy attenuated these alterations without fully restoring homeostasis. These results reveal that heatstroke occurs despite a robust HSR and is associated with severe alteration of proteostasis, bioenergetics, and DNA instability.

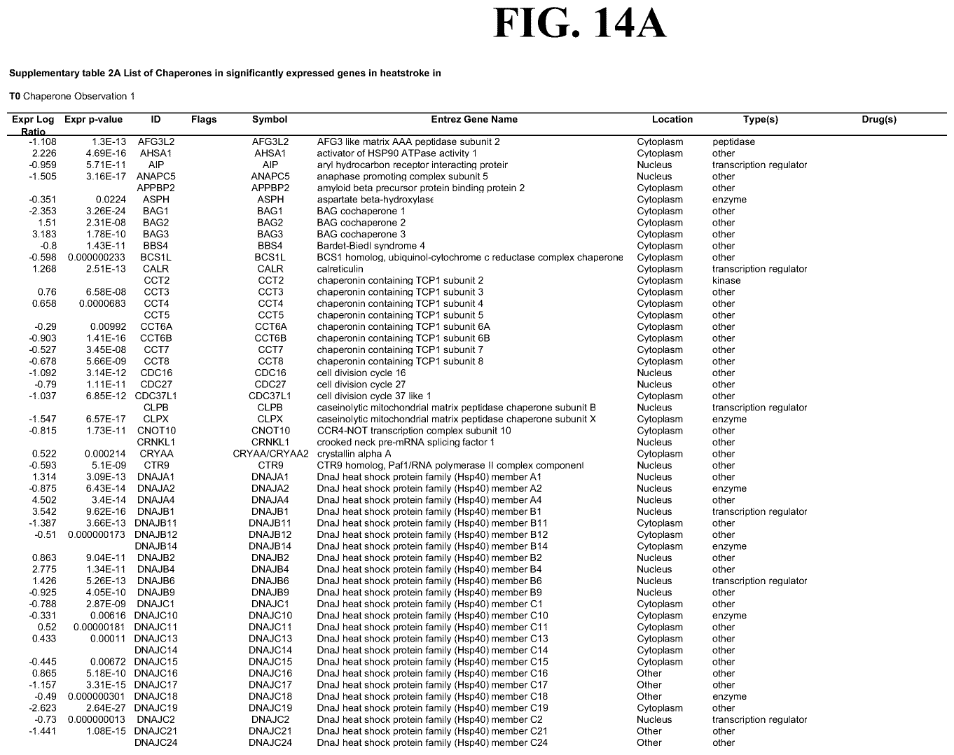

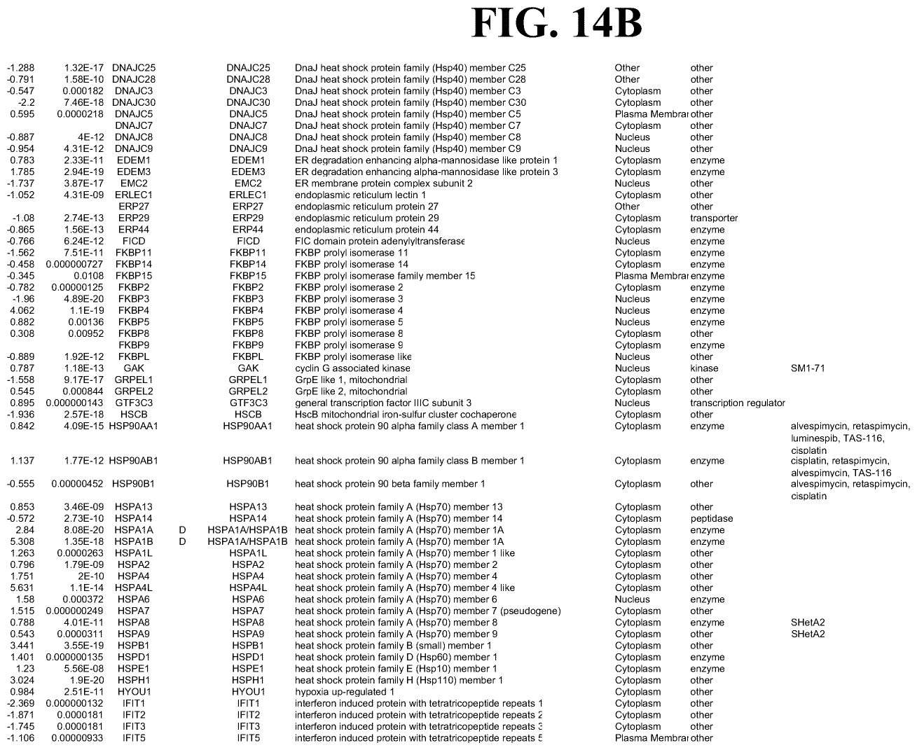

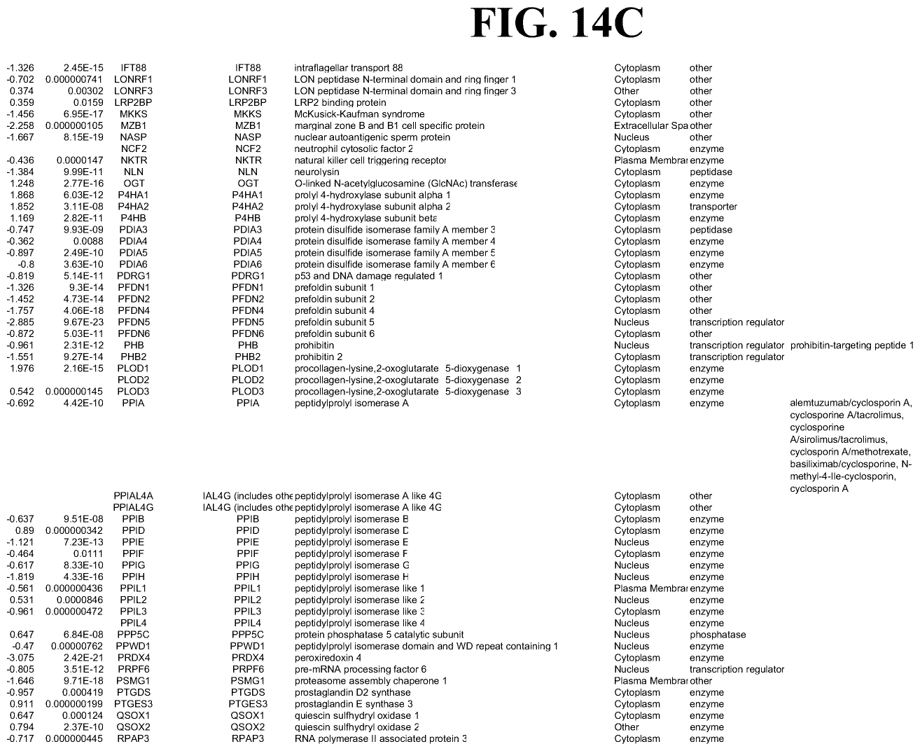

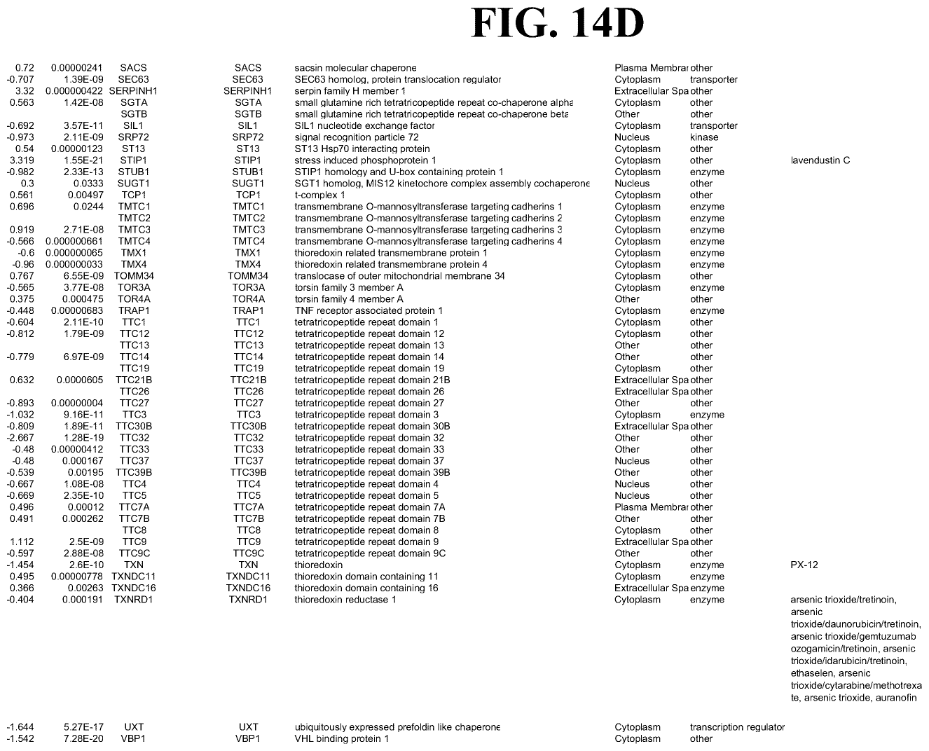

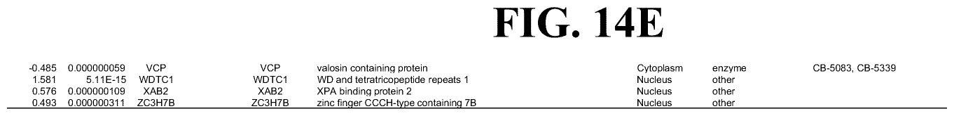

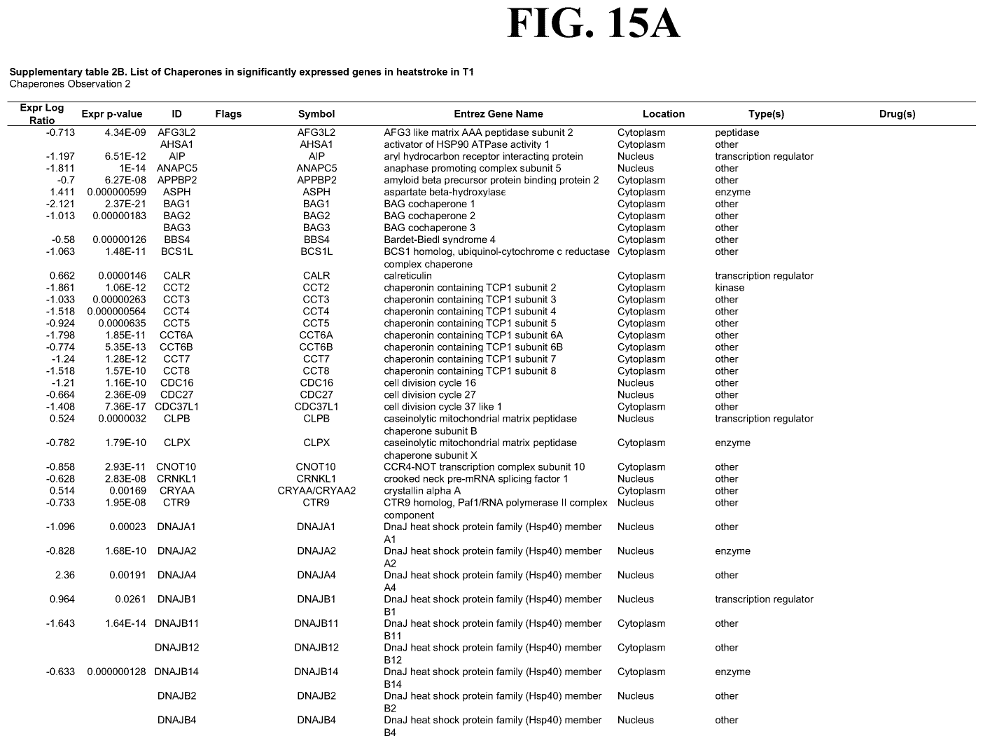

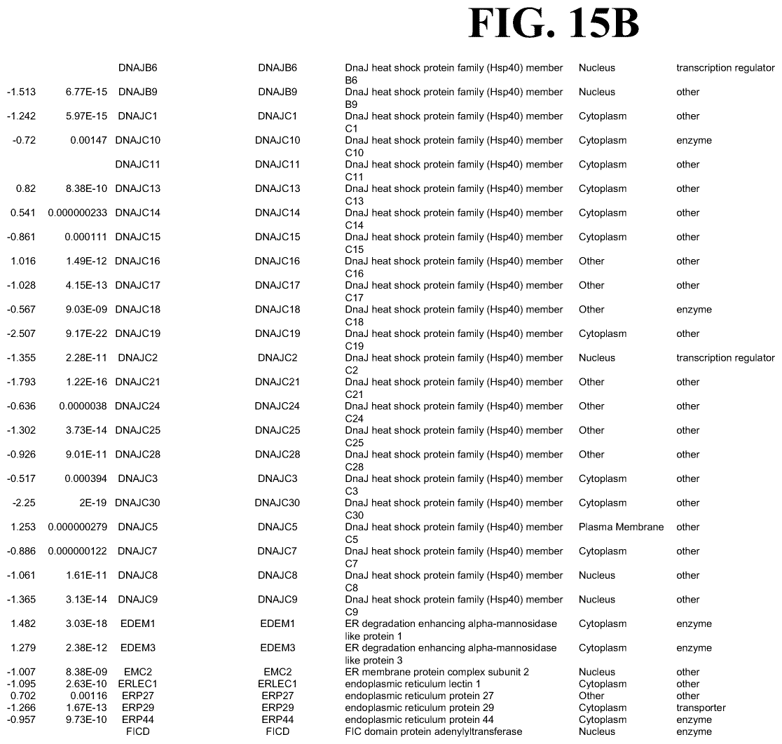

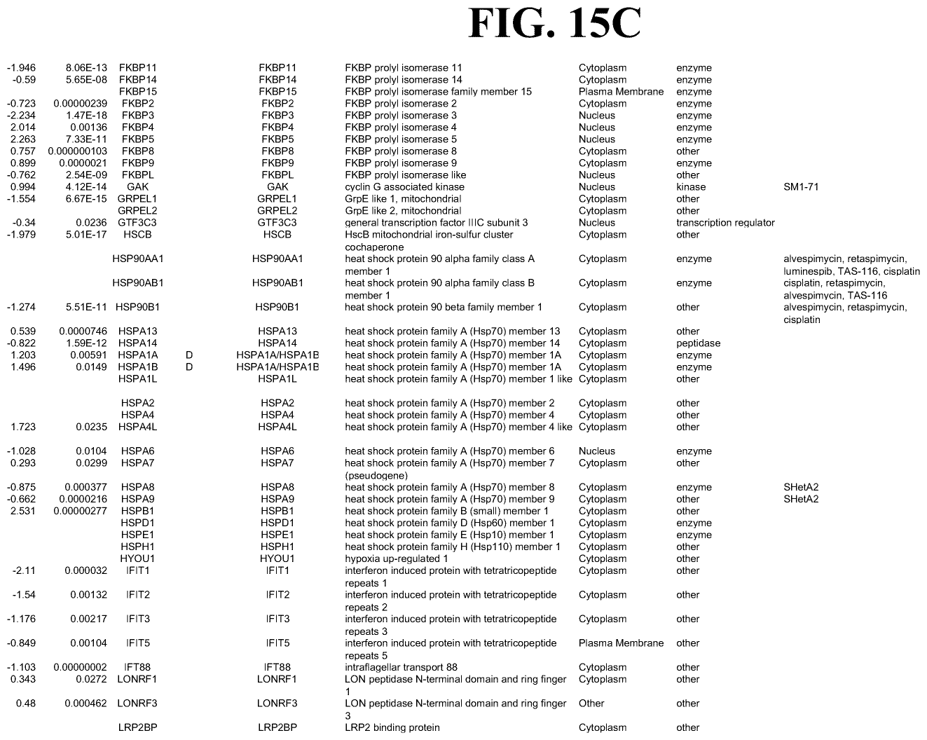

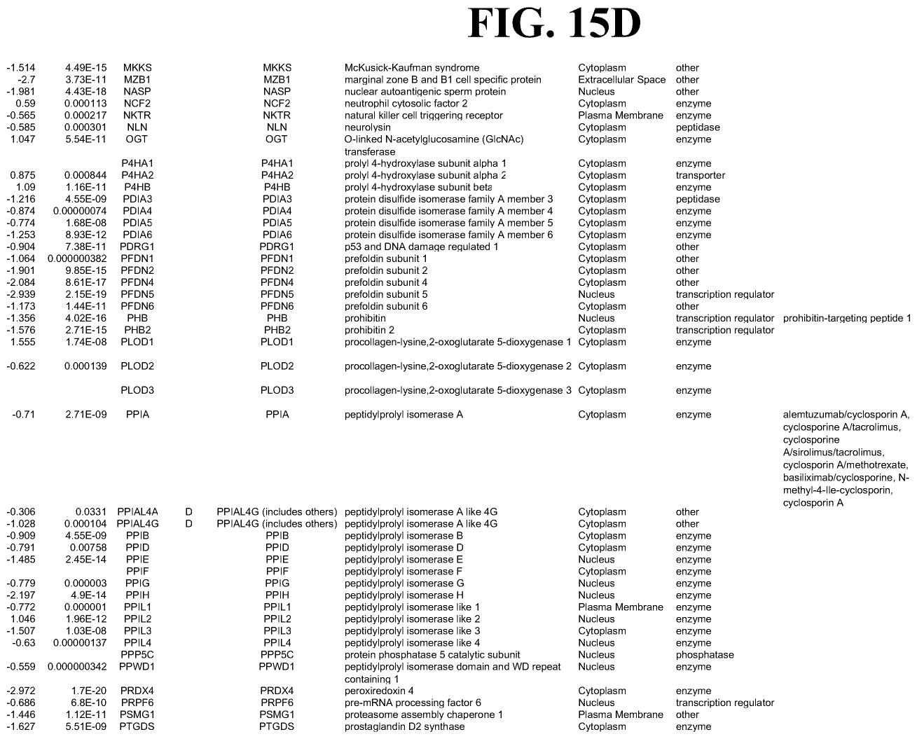

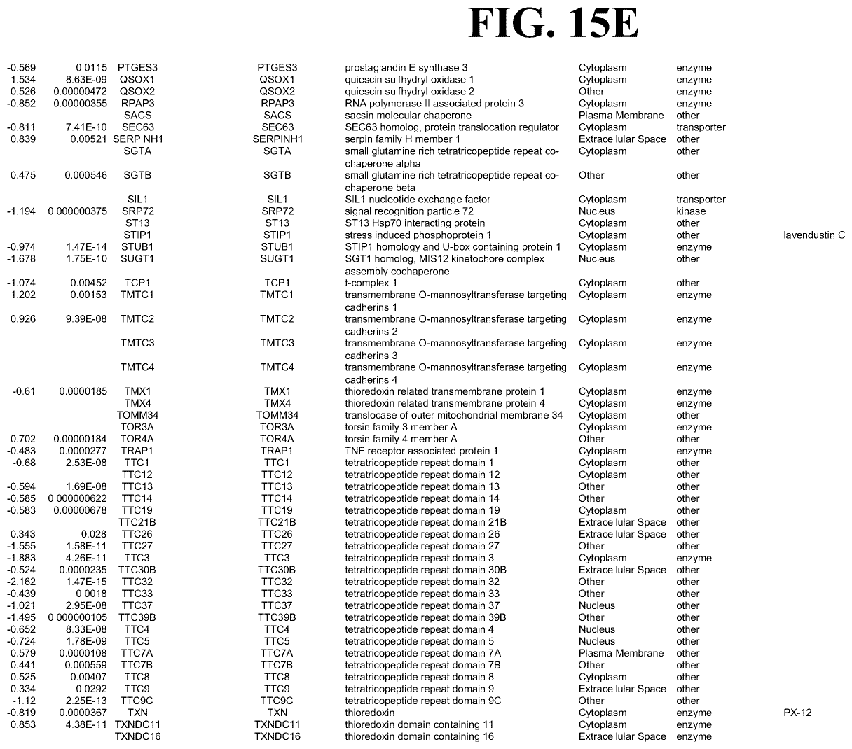

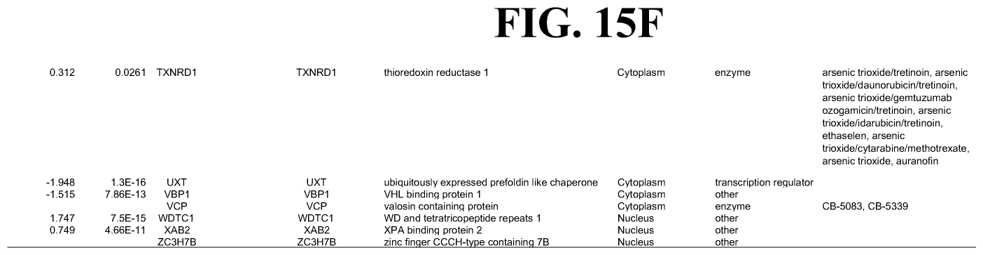

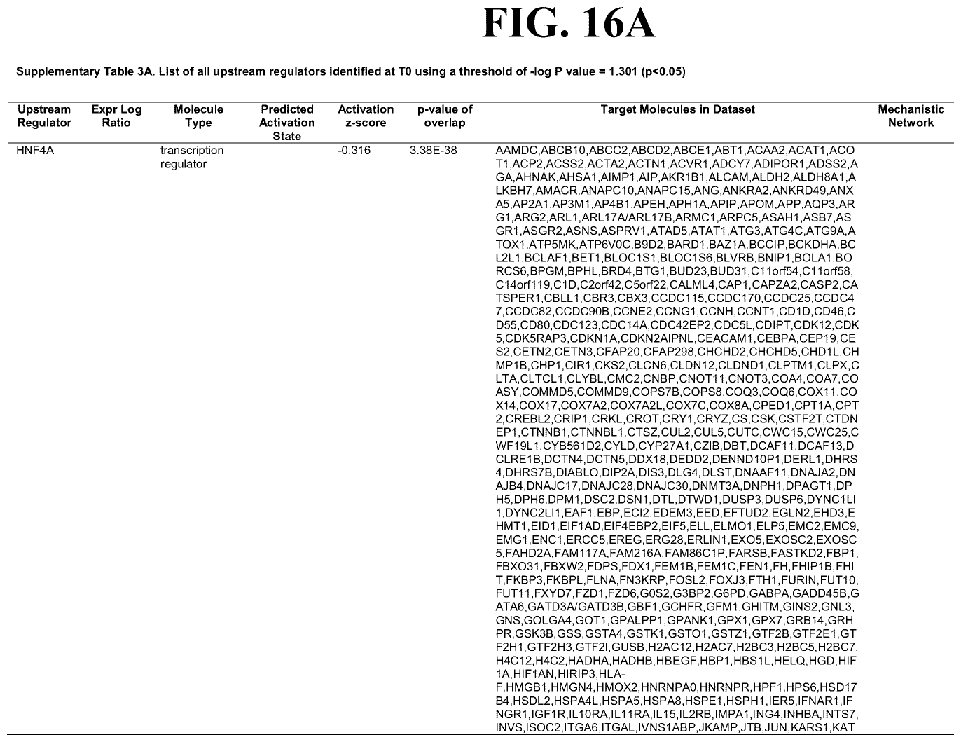

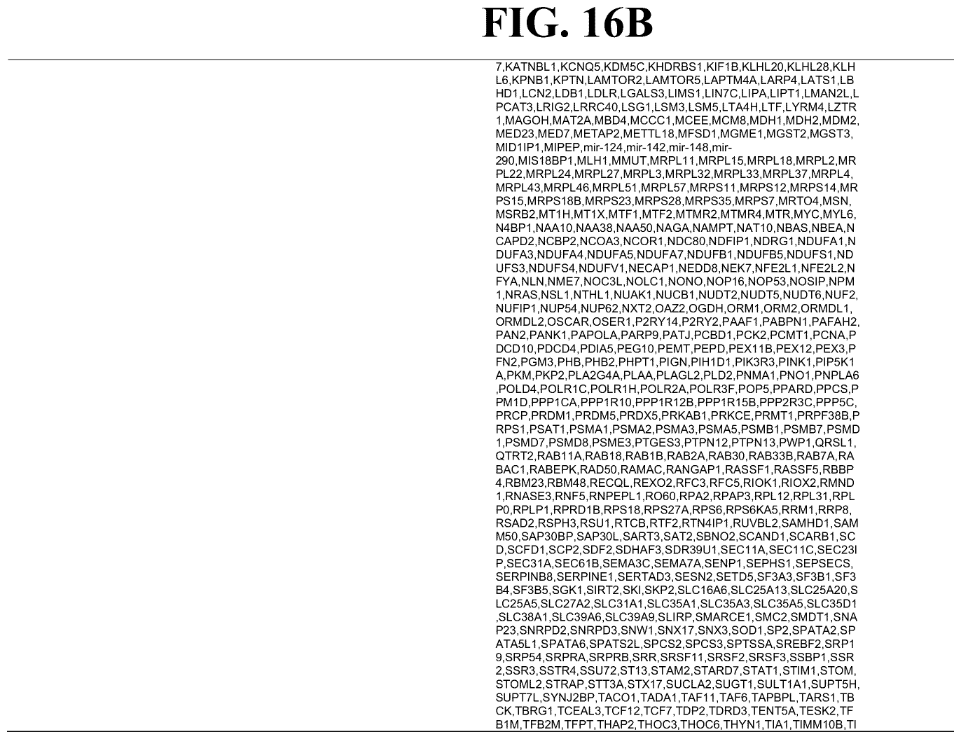

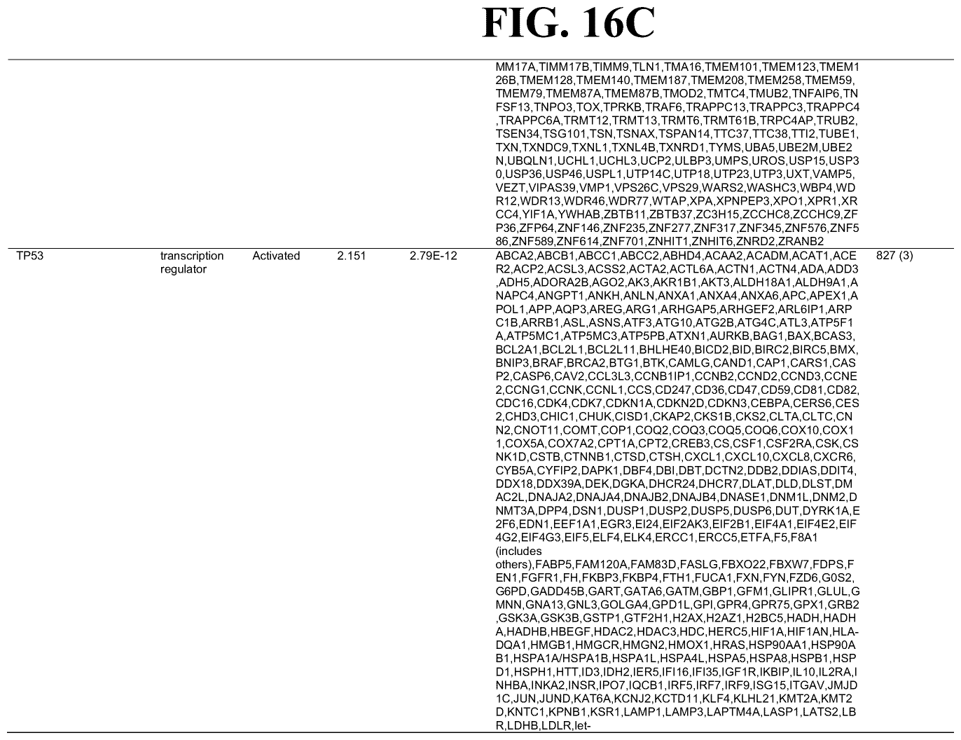

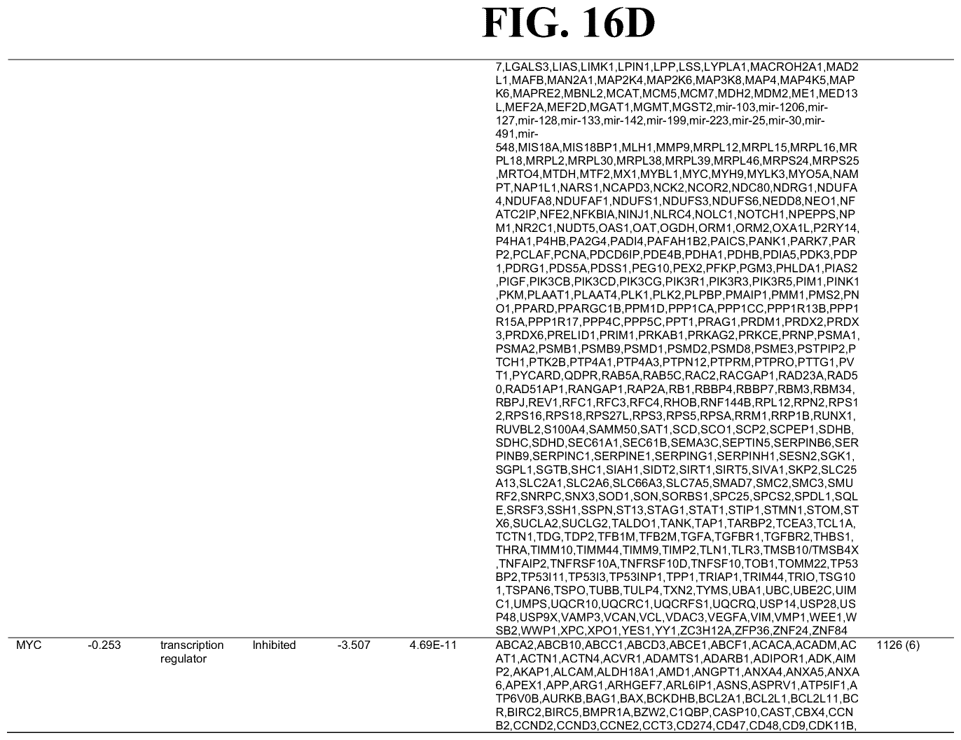

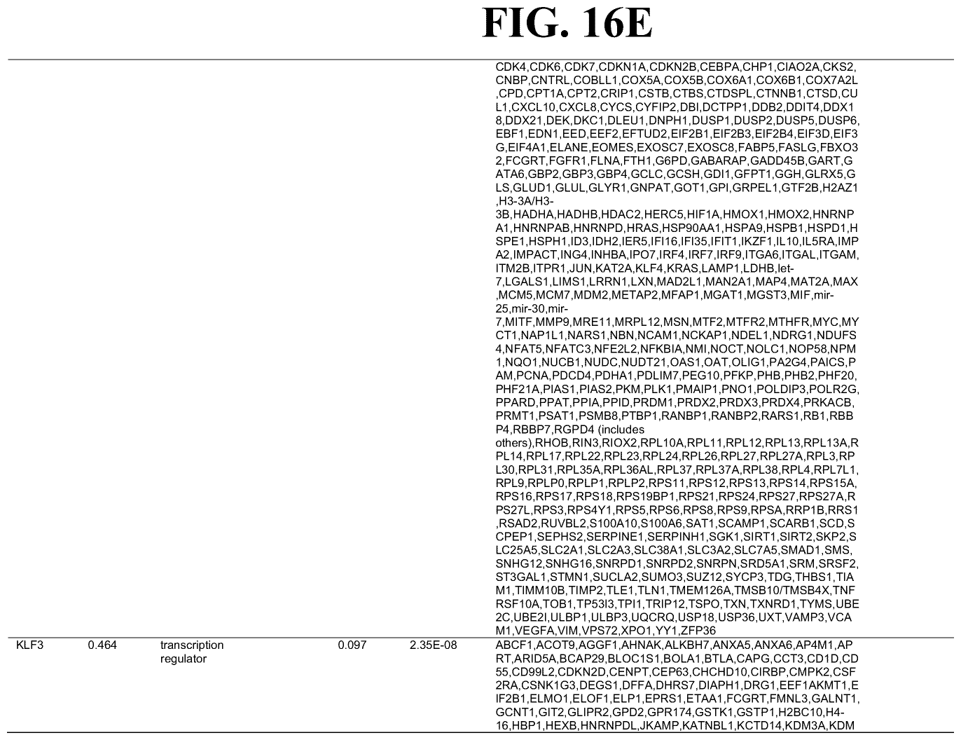

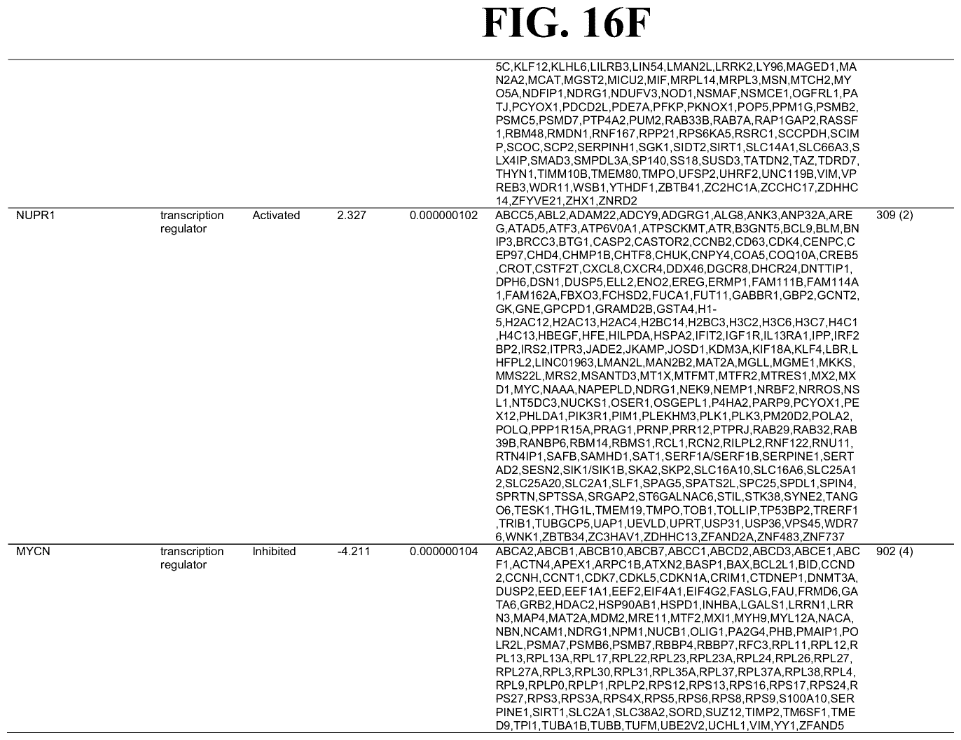

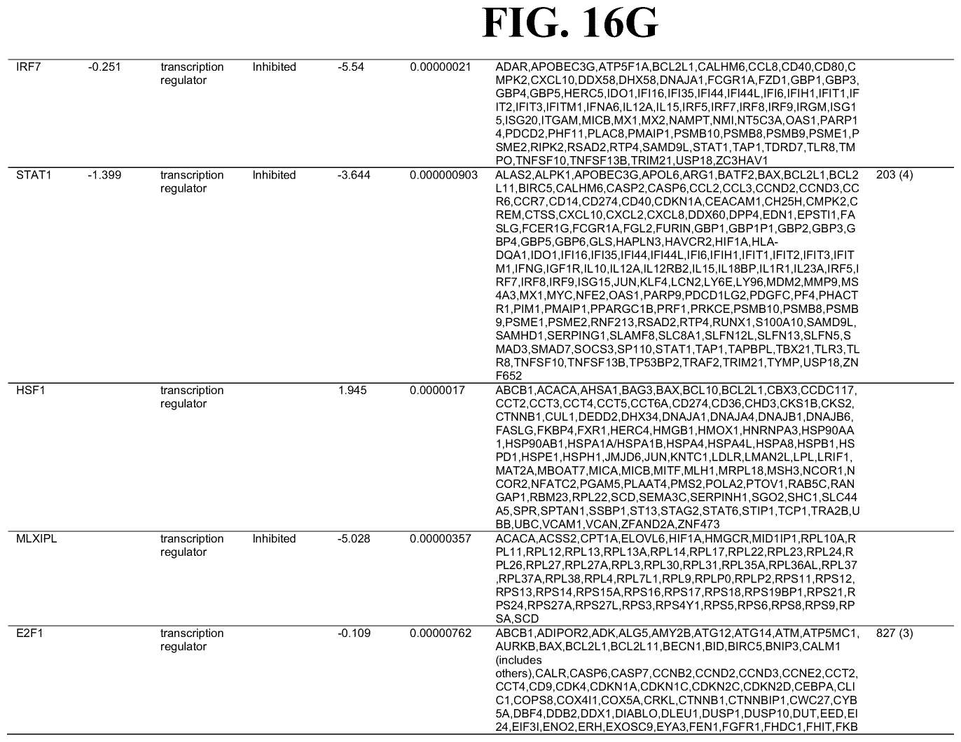

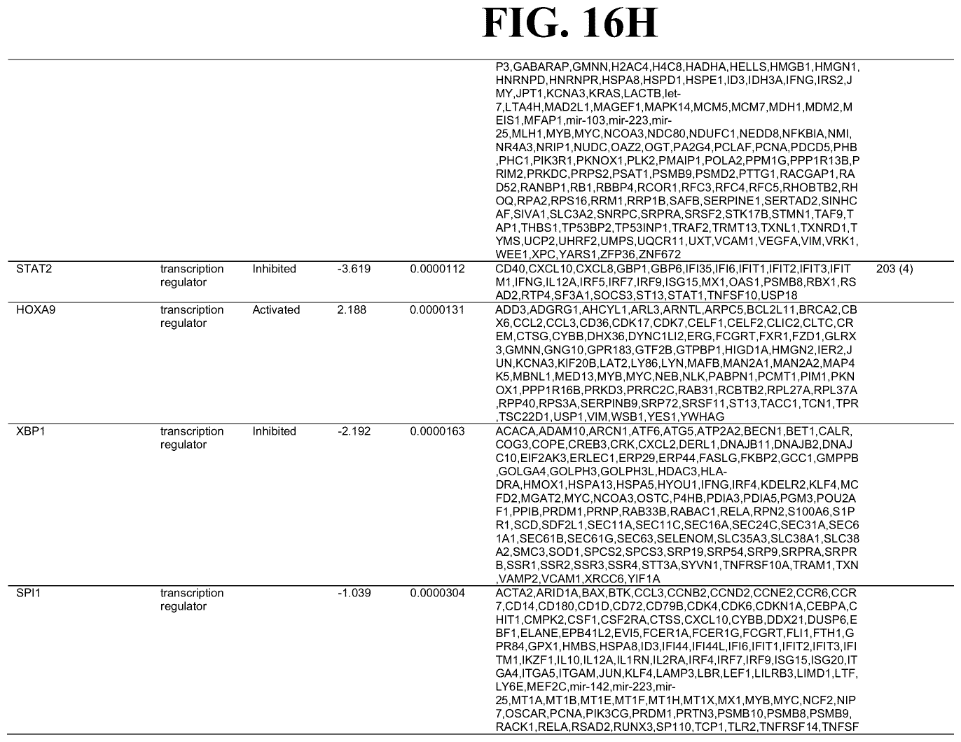

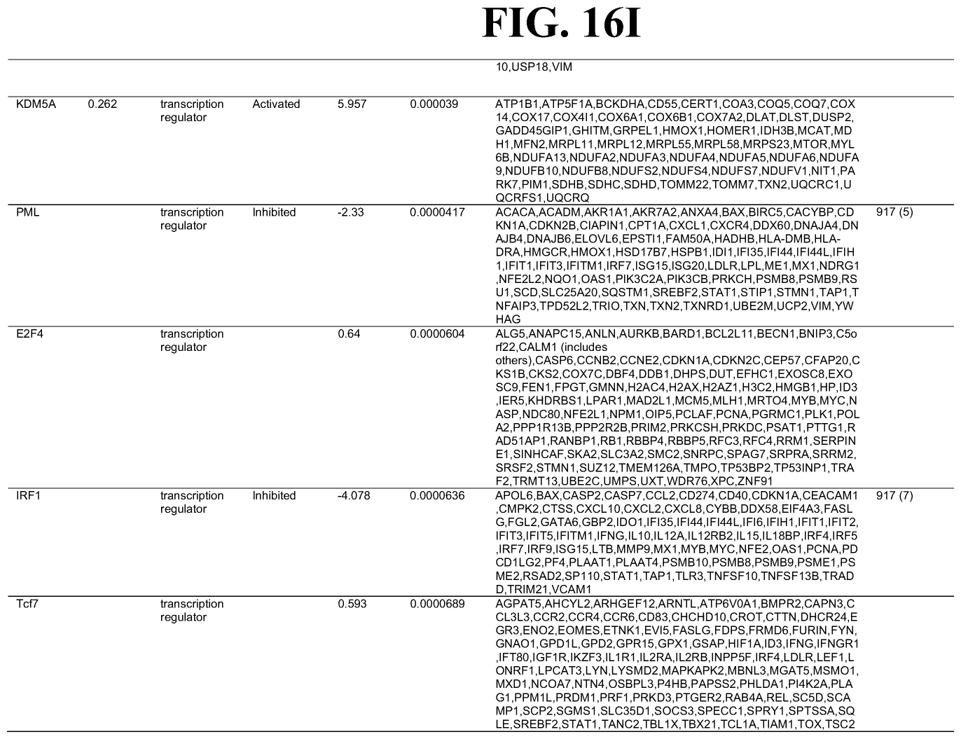

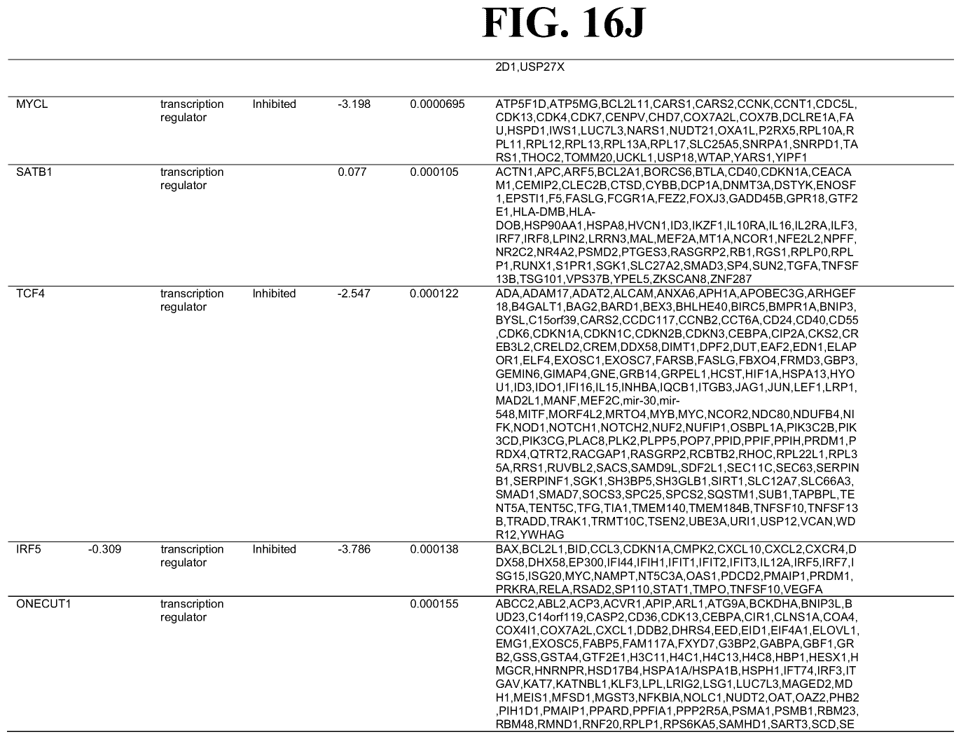

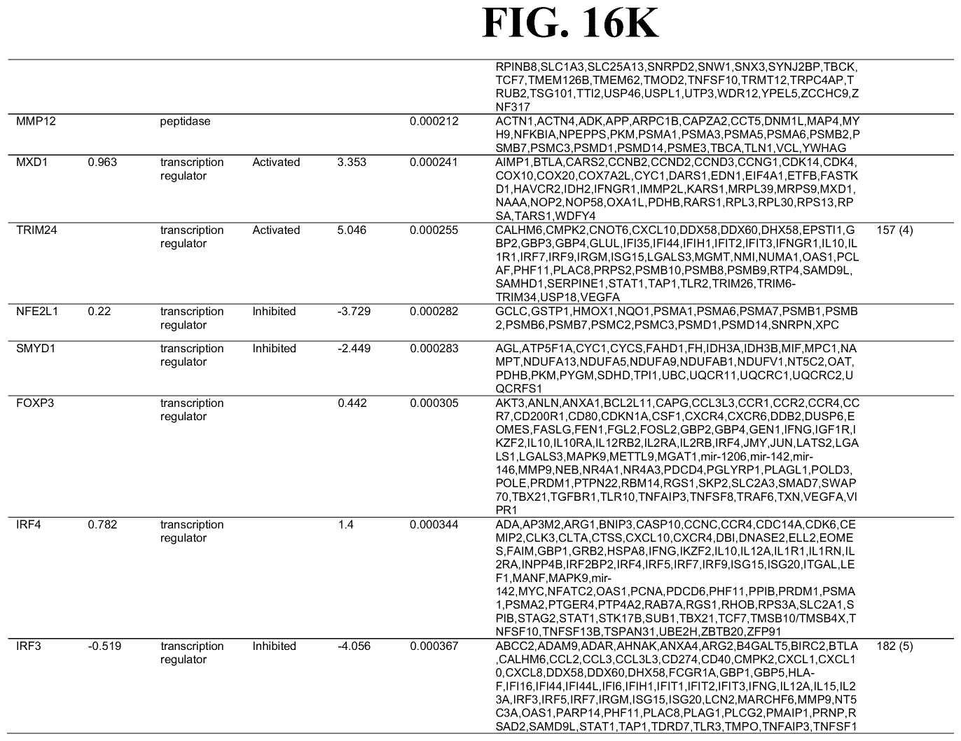

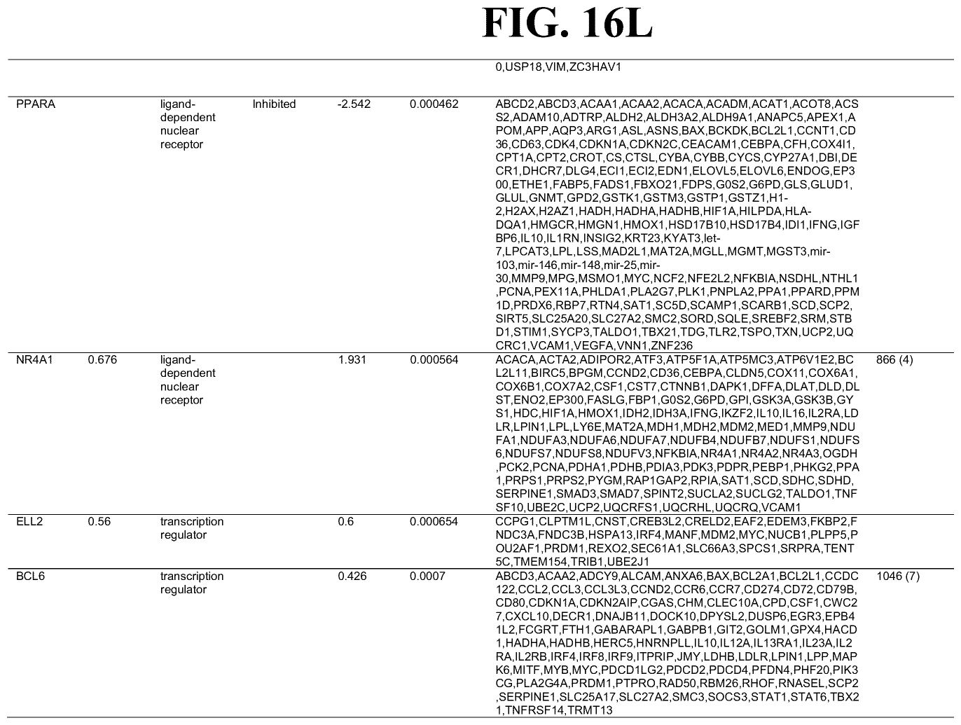

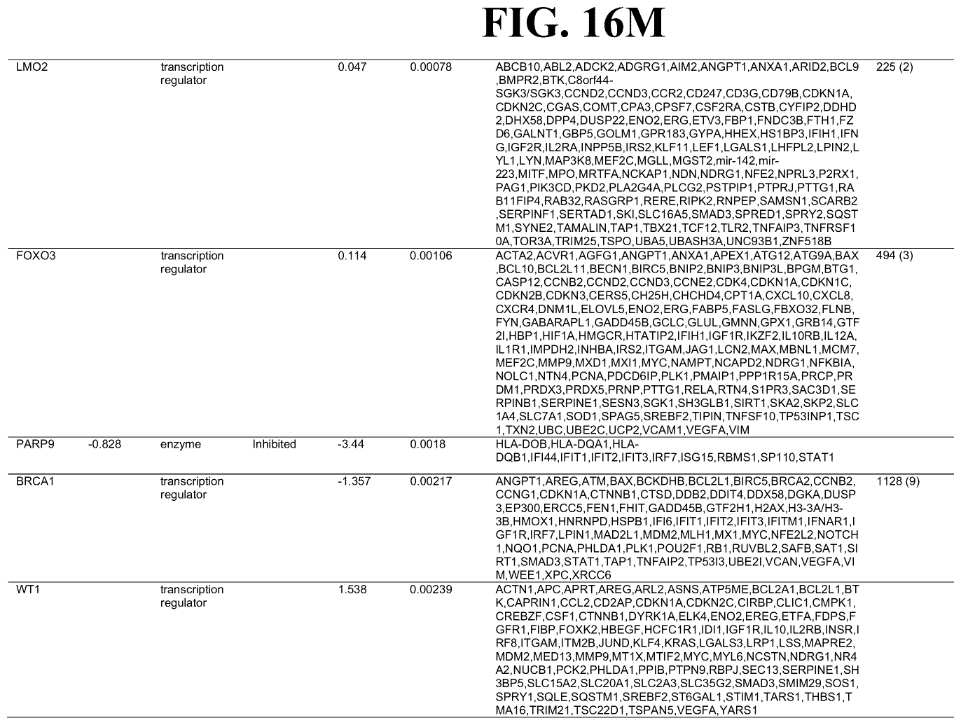

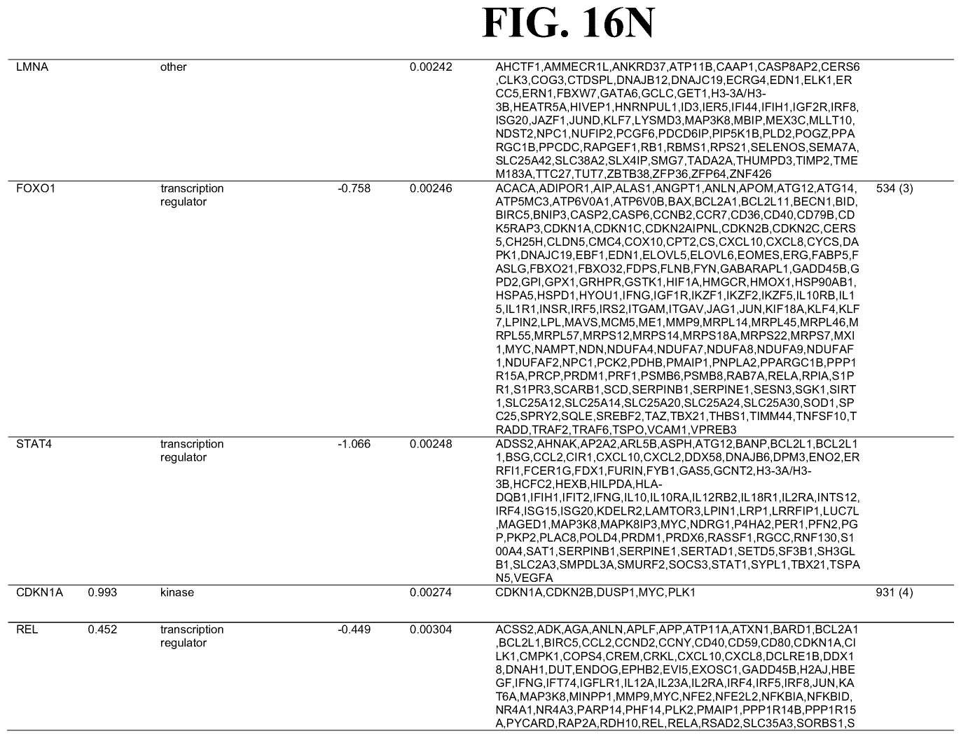

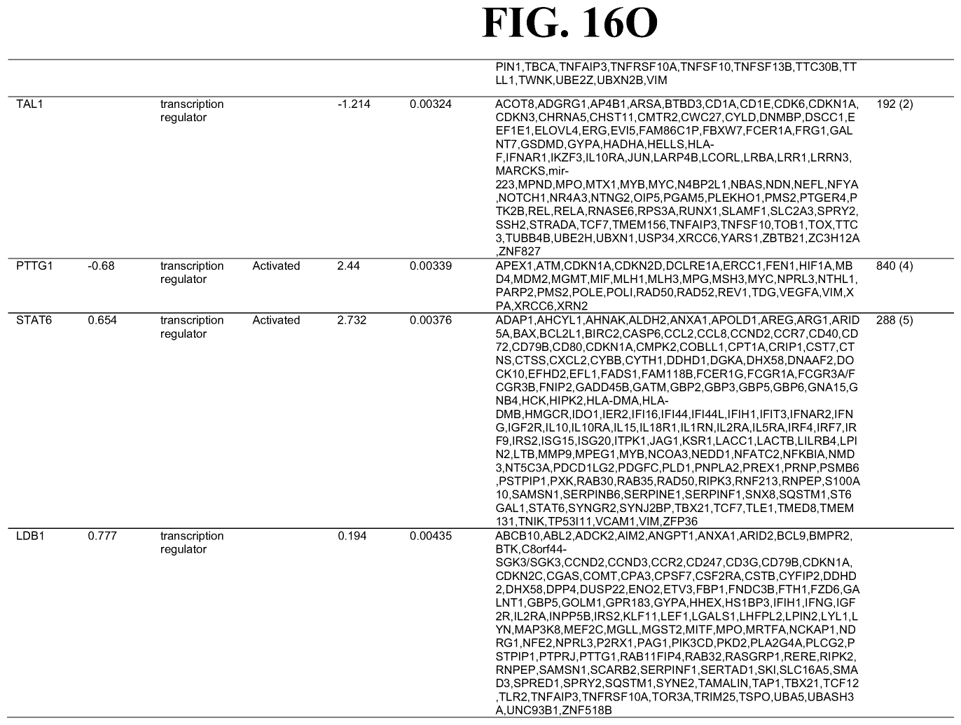

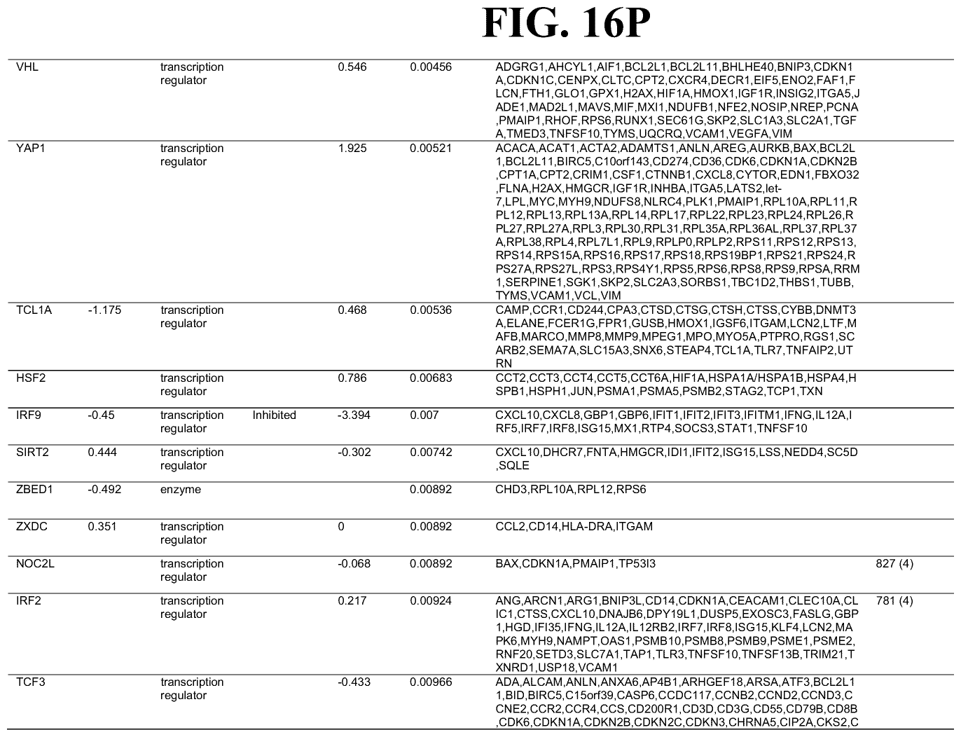

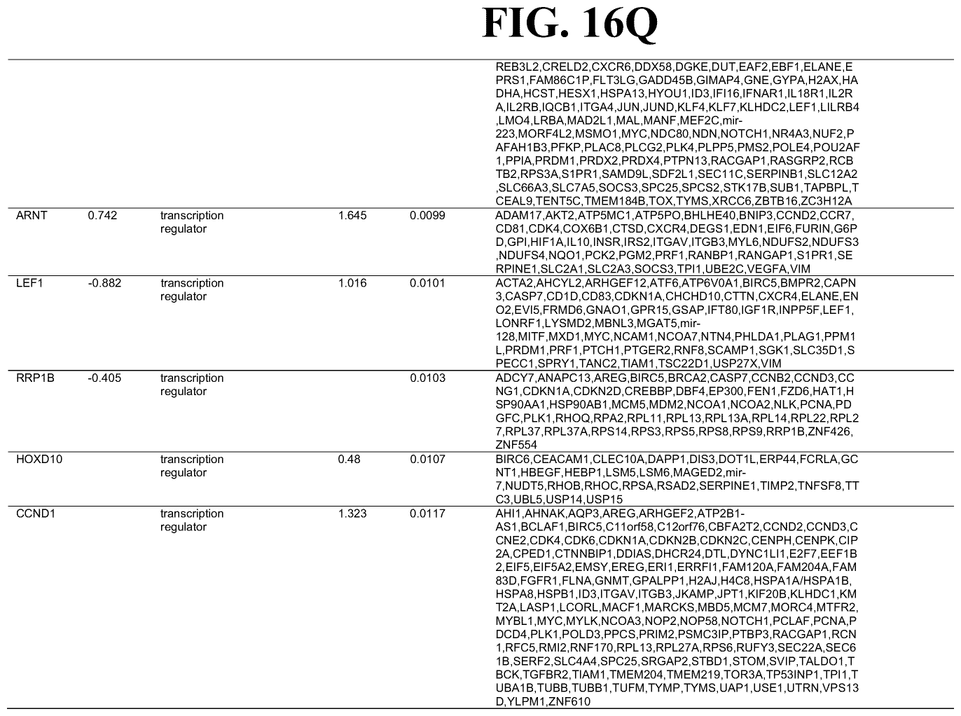

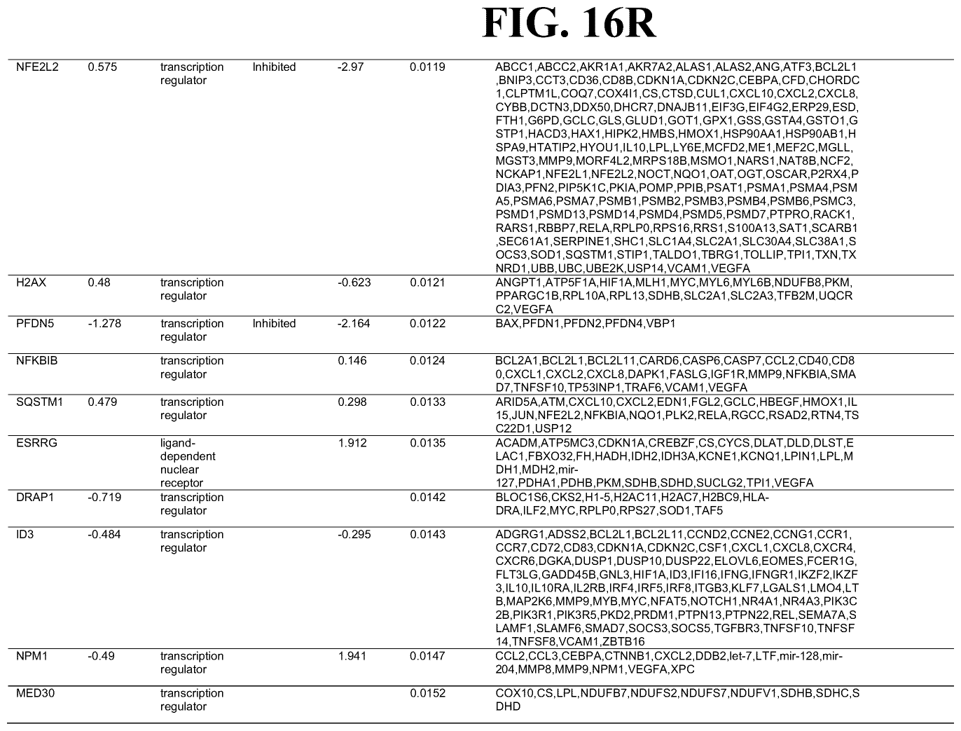

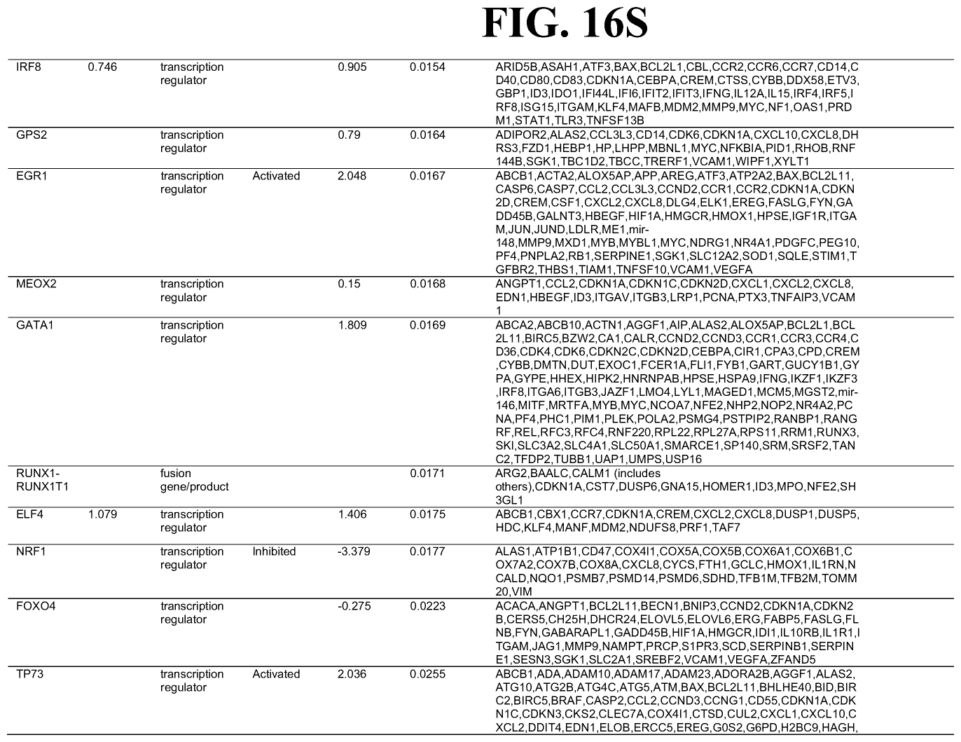

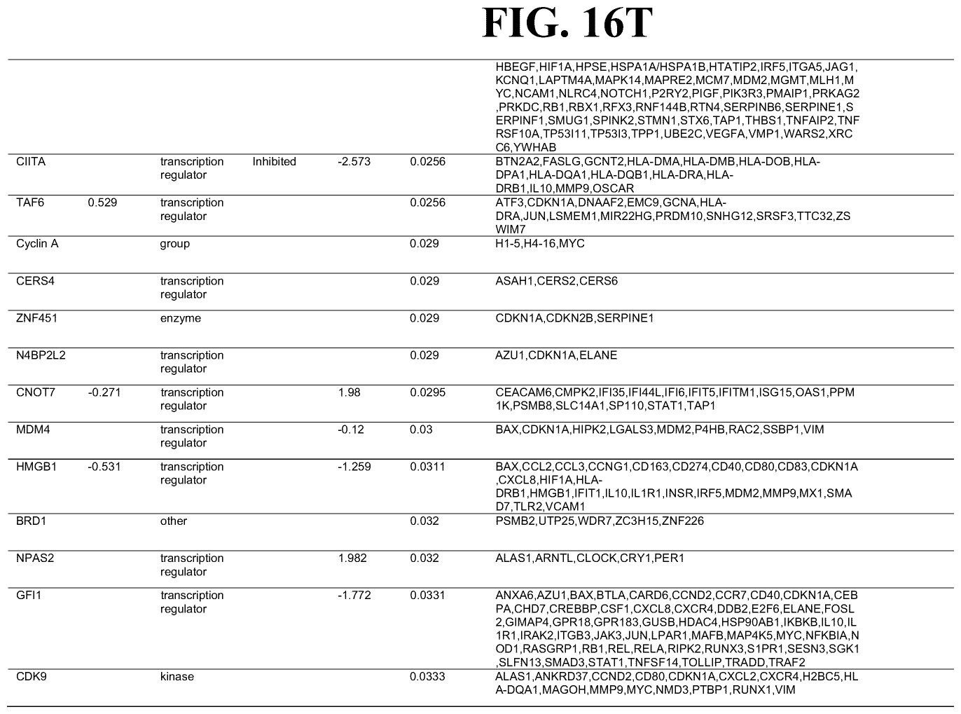

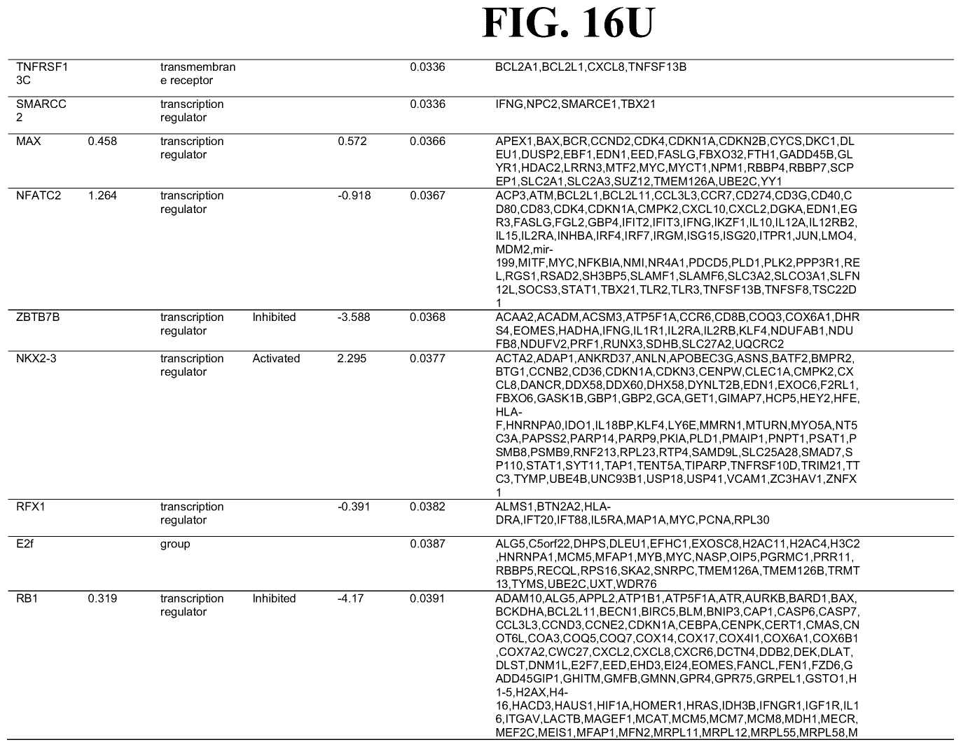

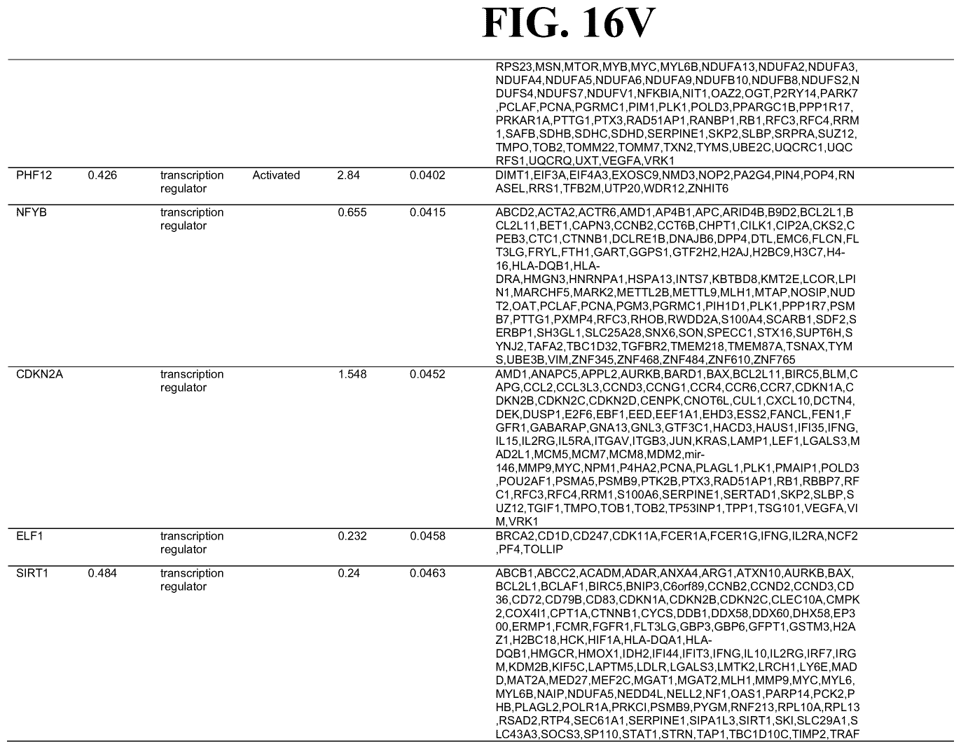

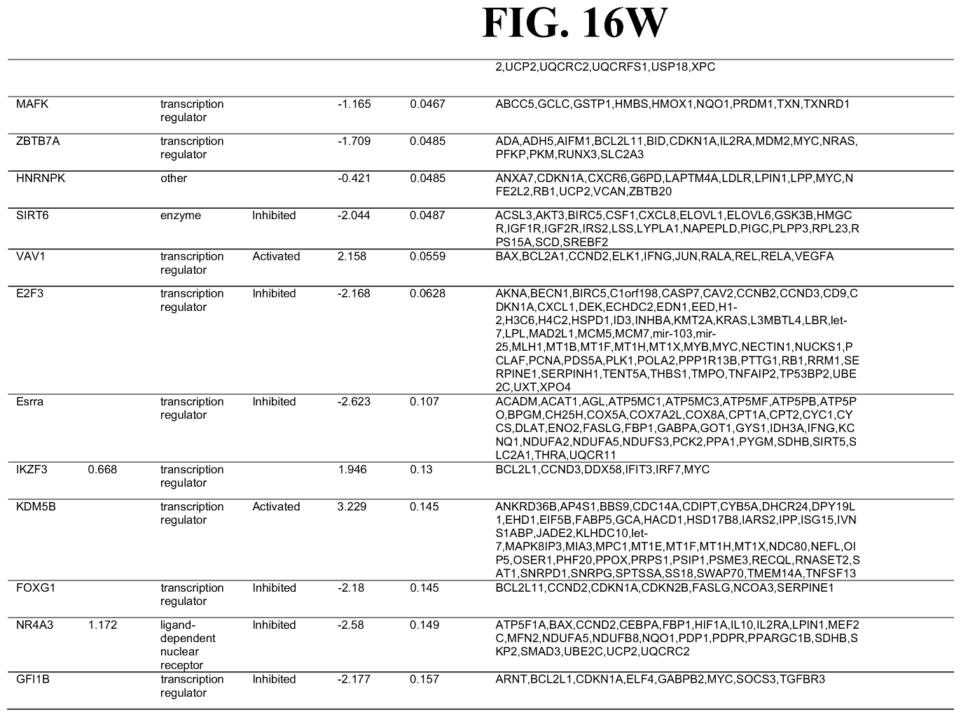

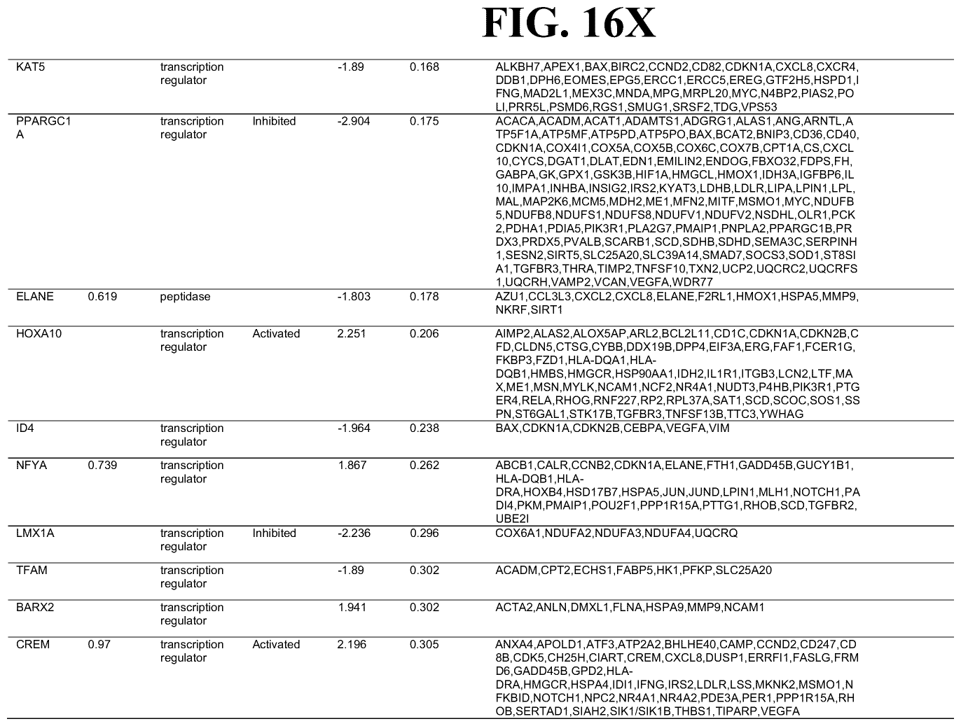

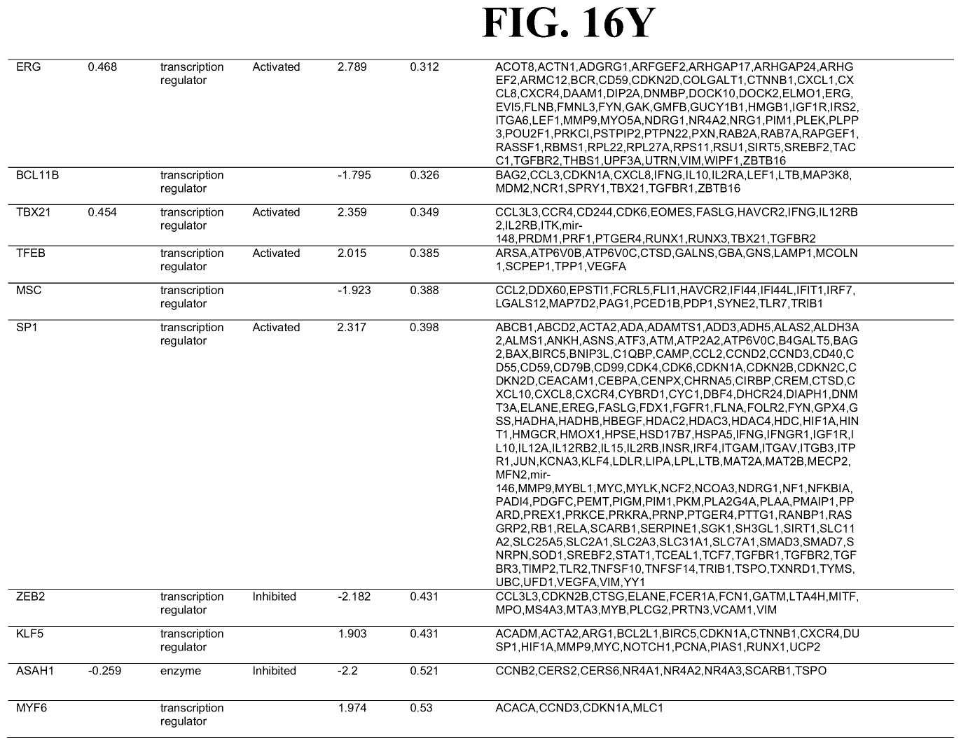

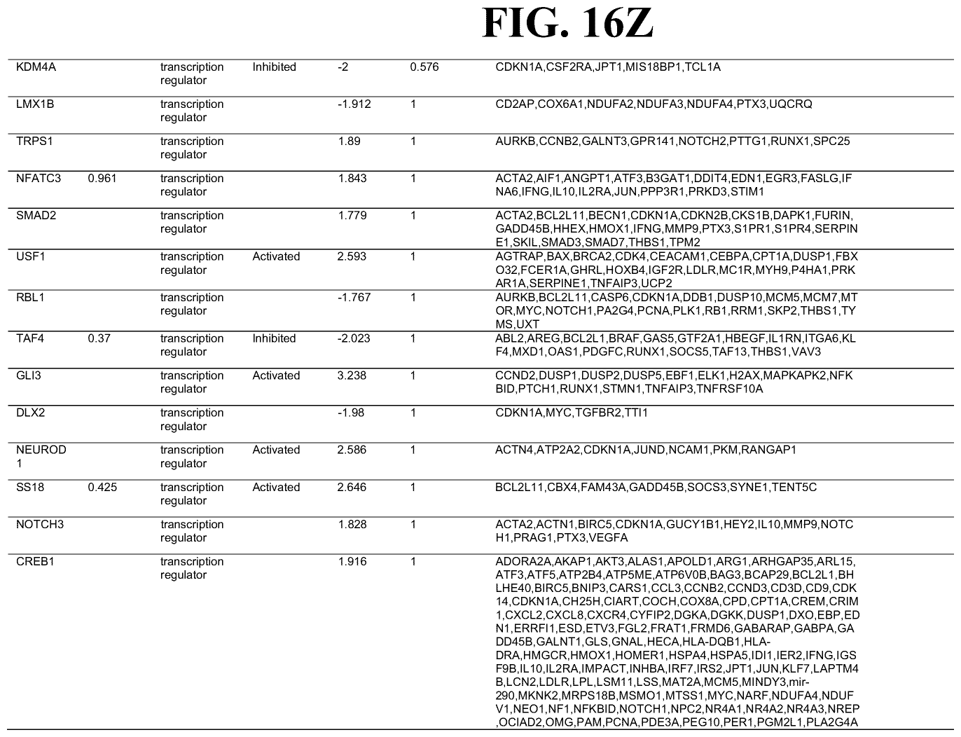

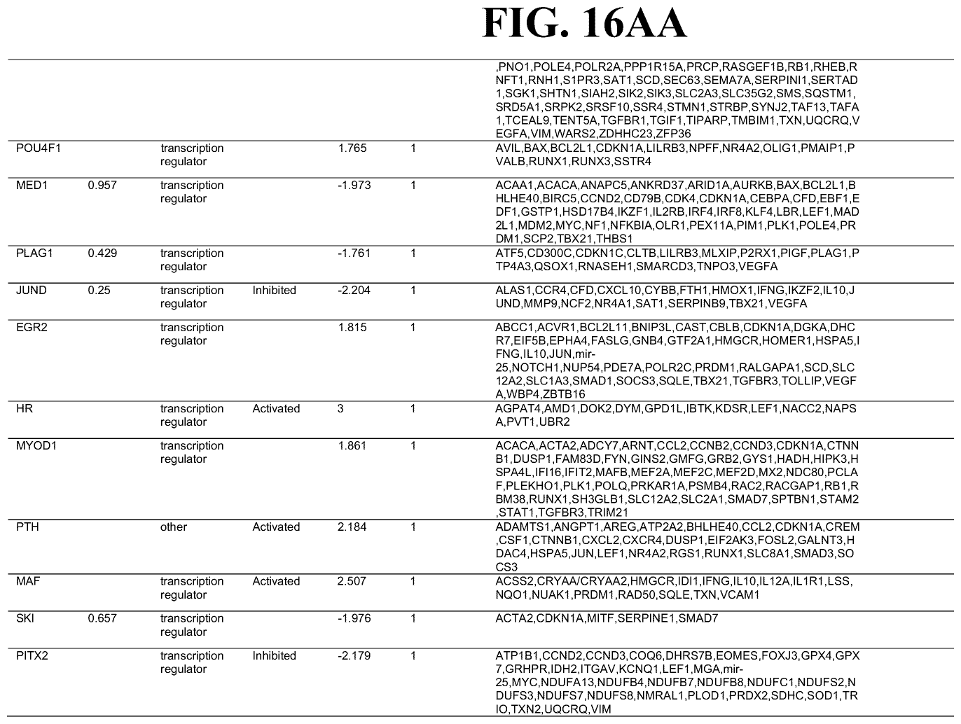

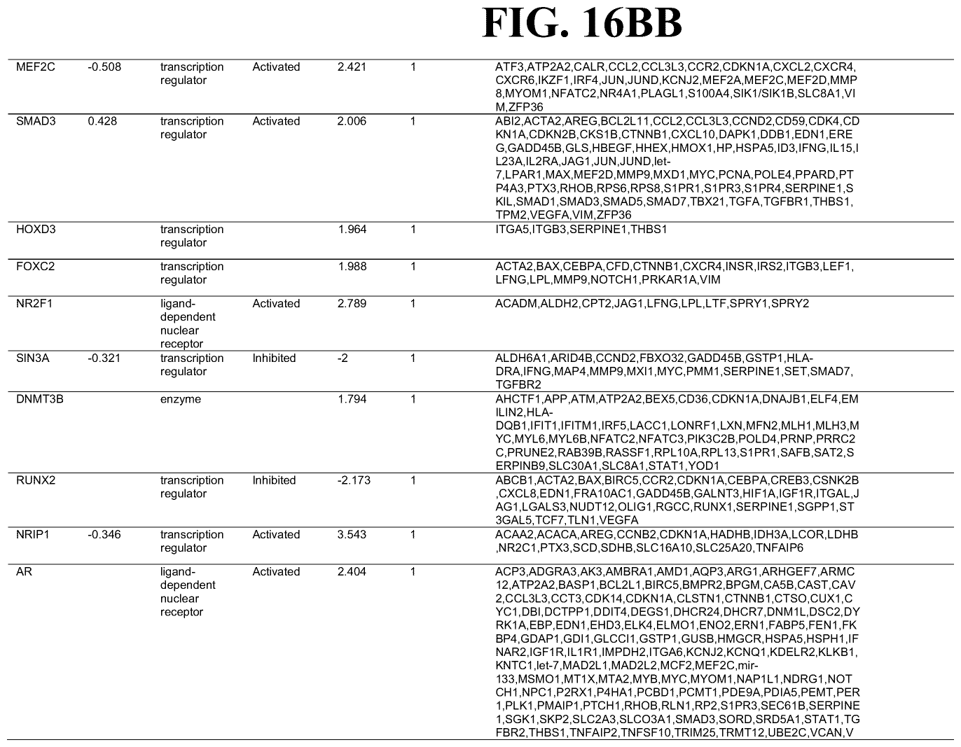

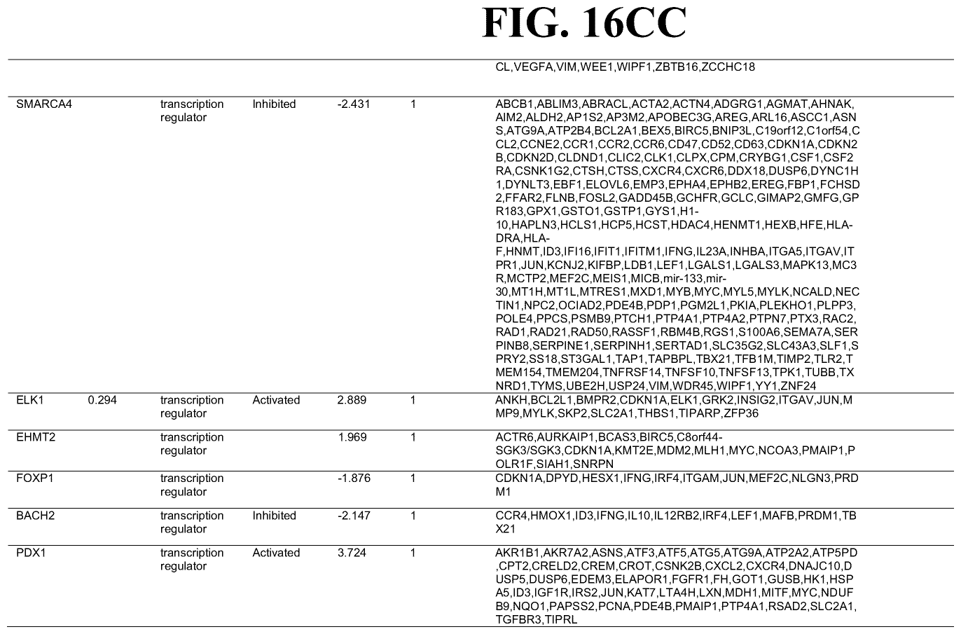

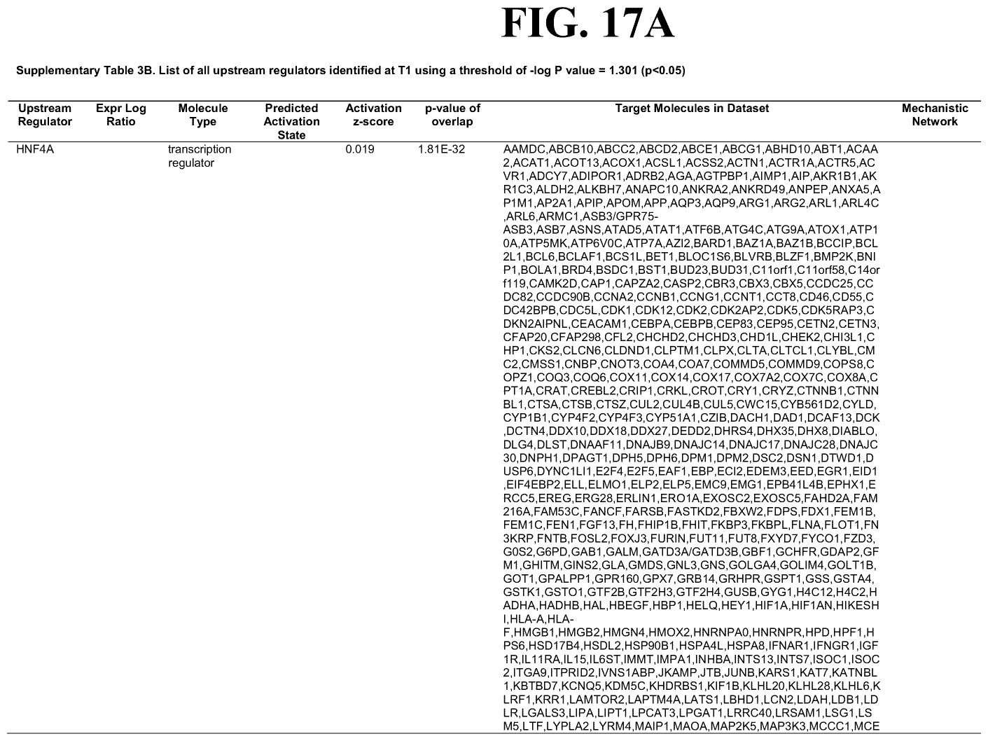

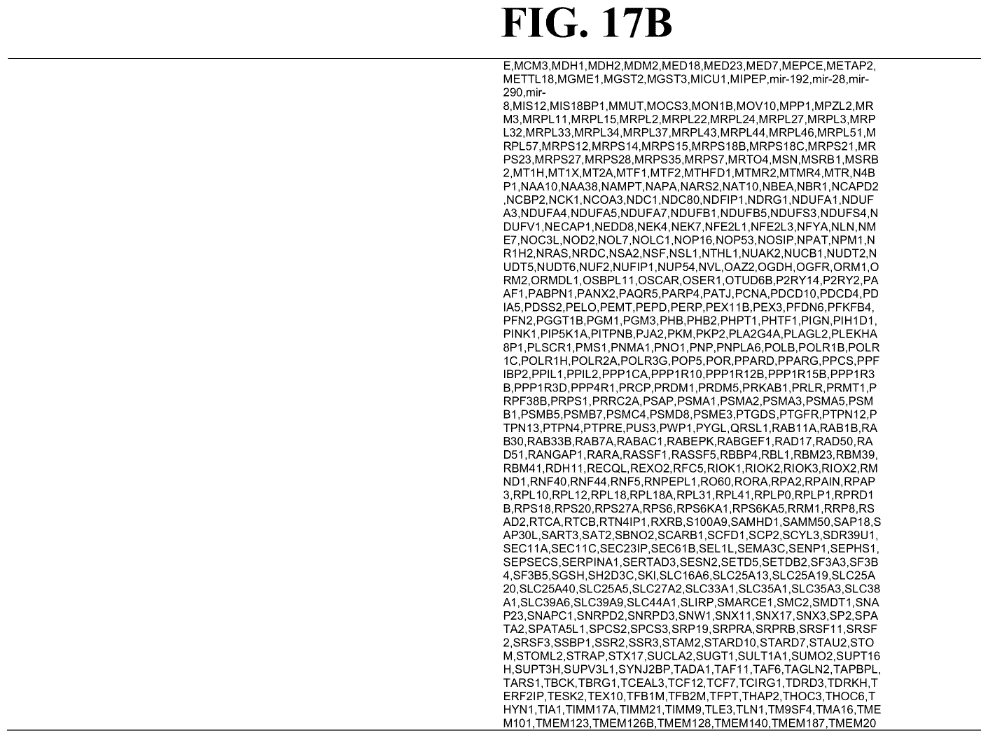

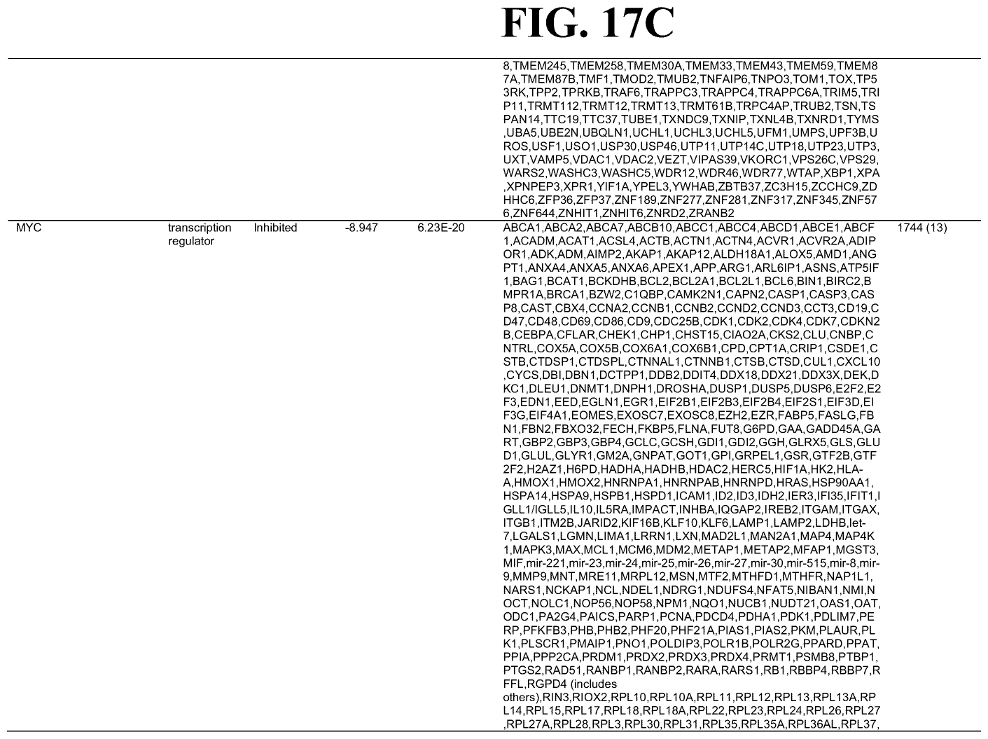

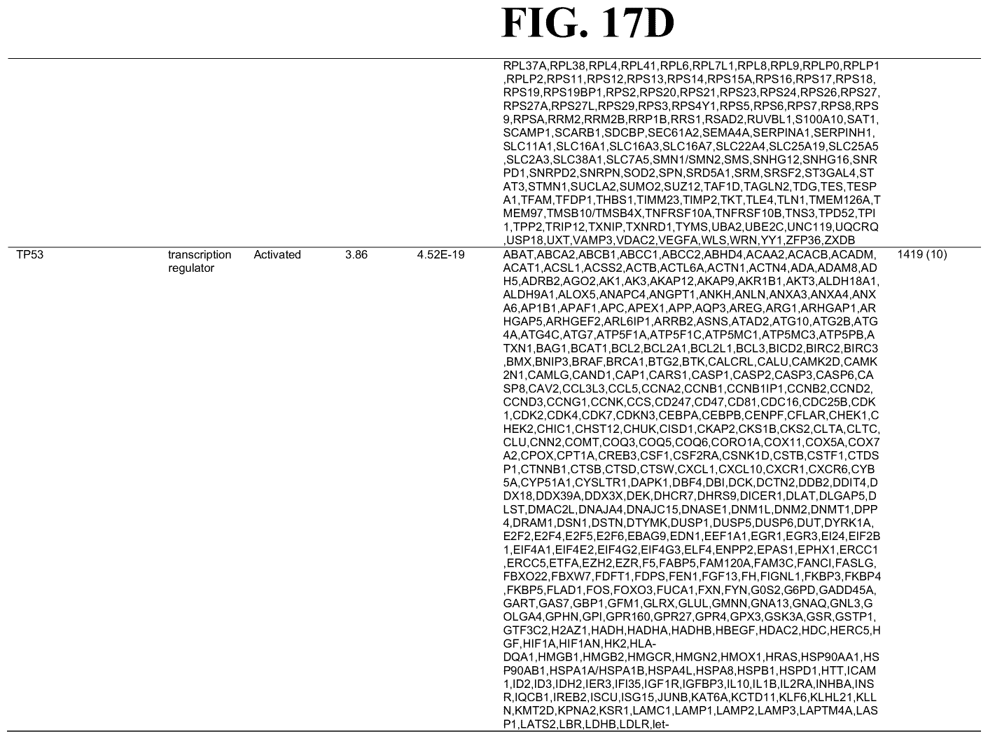

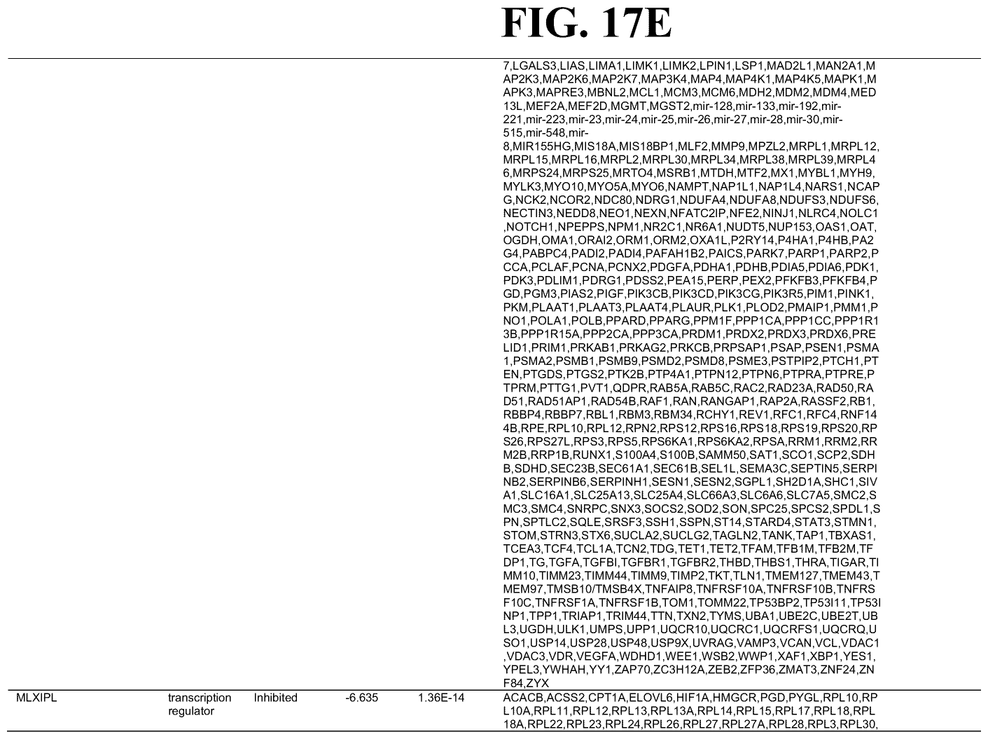

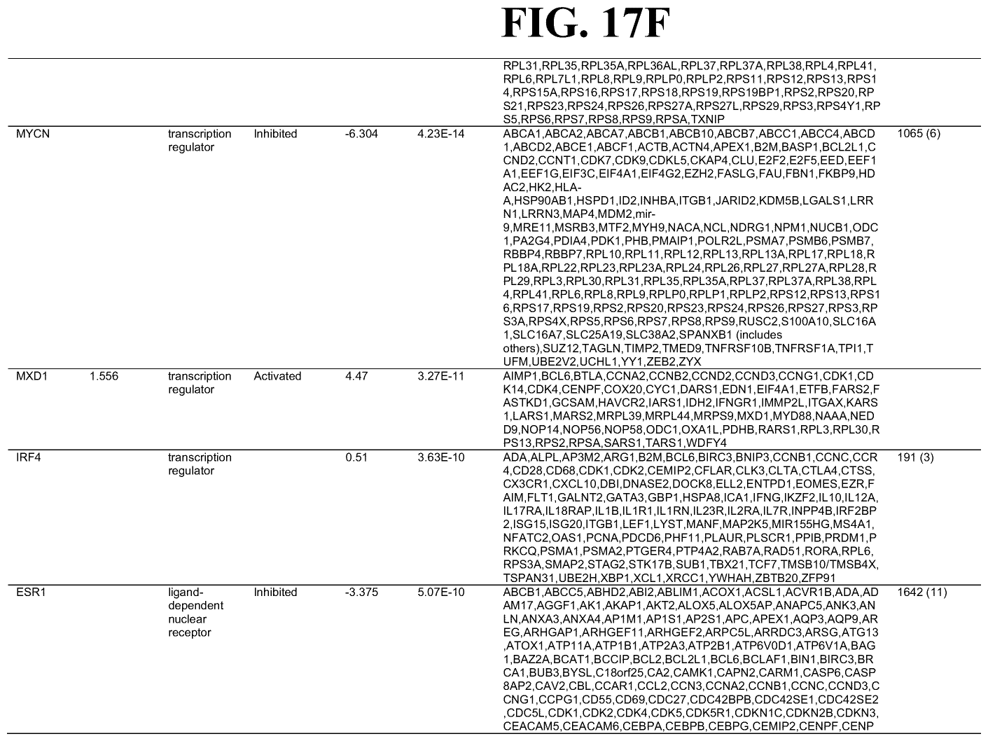

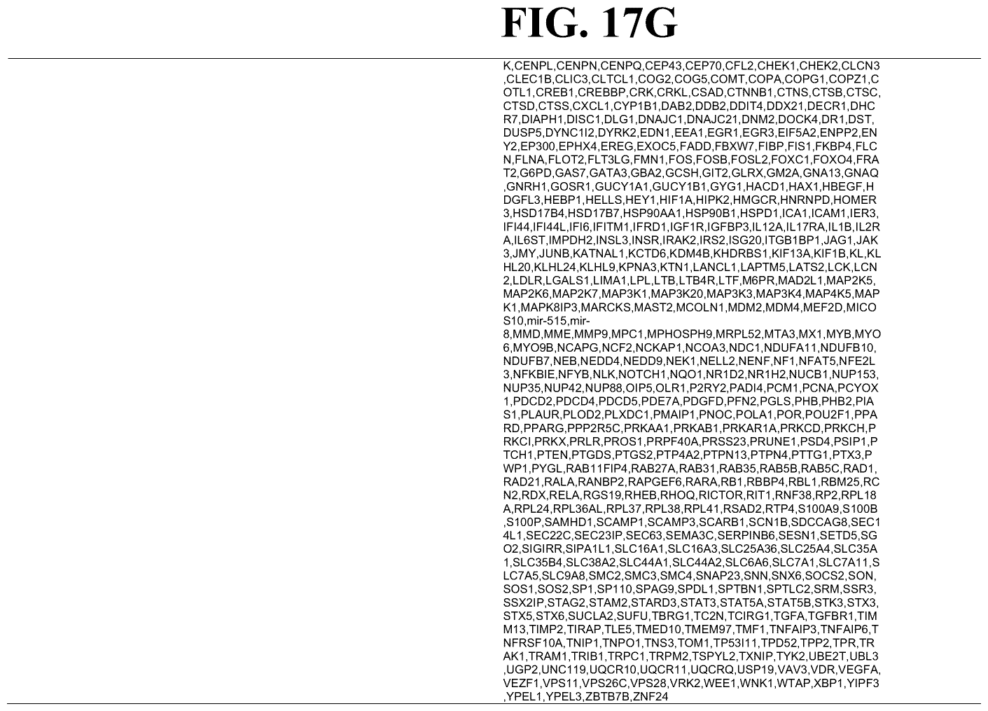

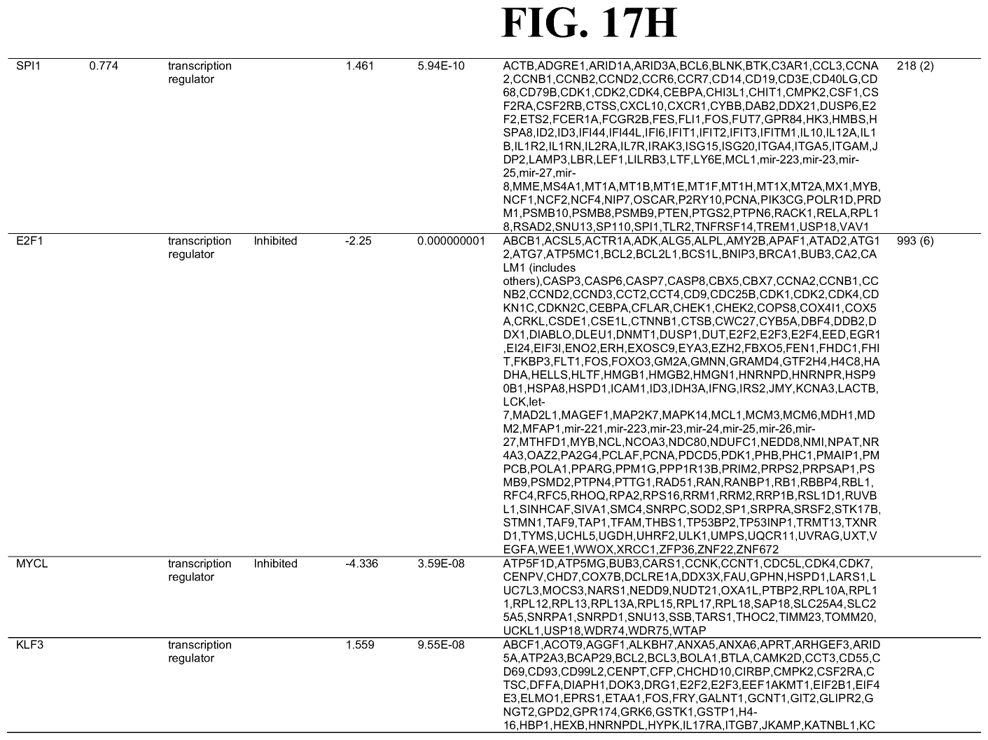

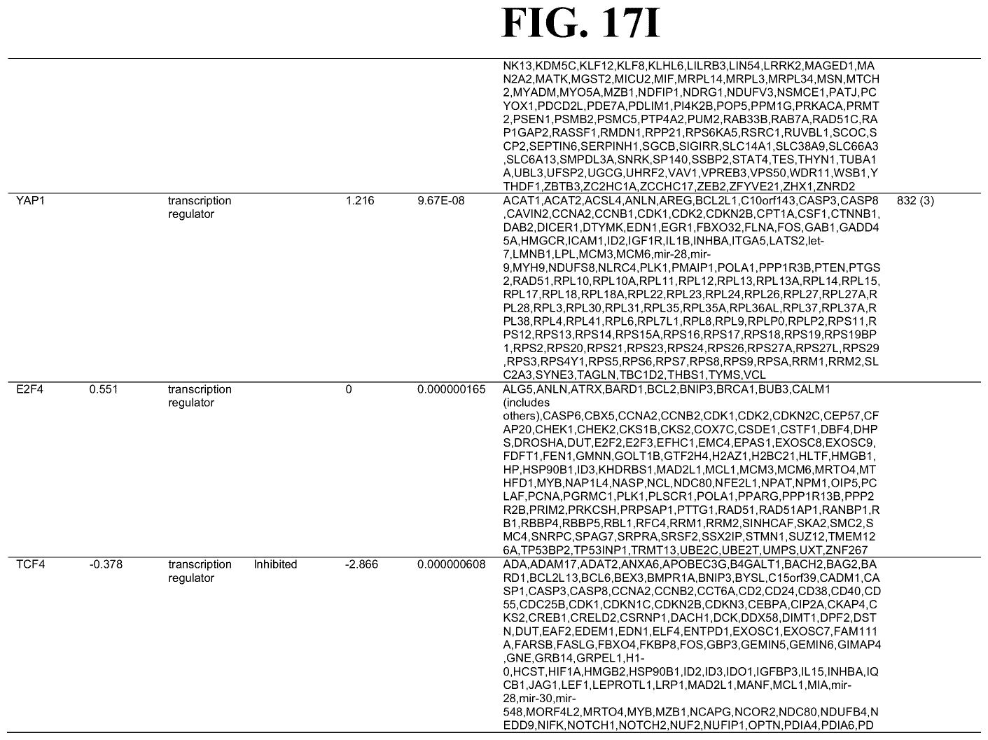

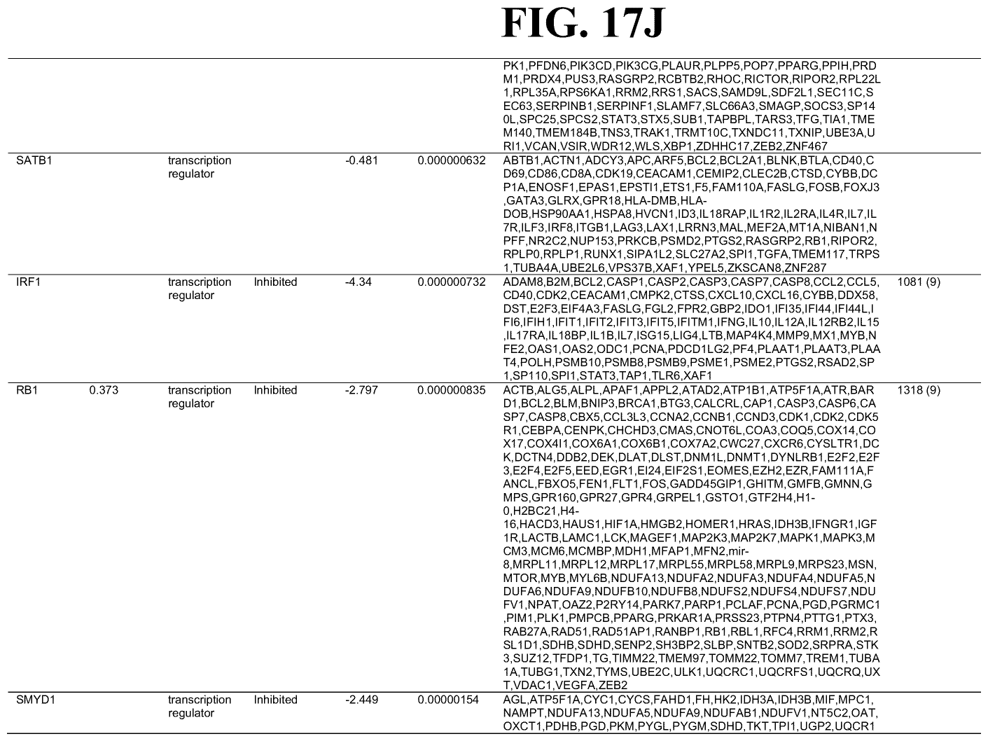

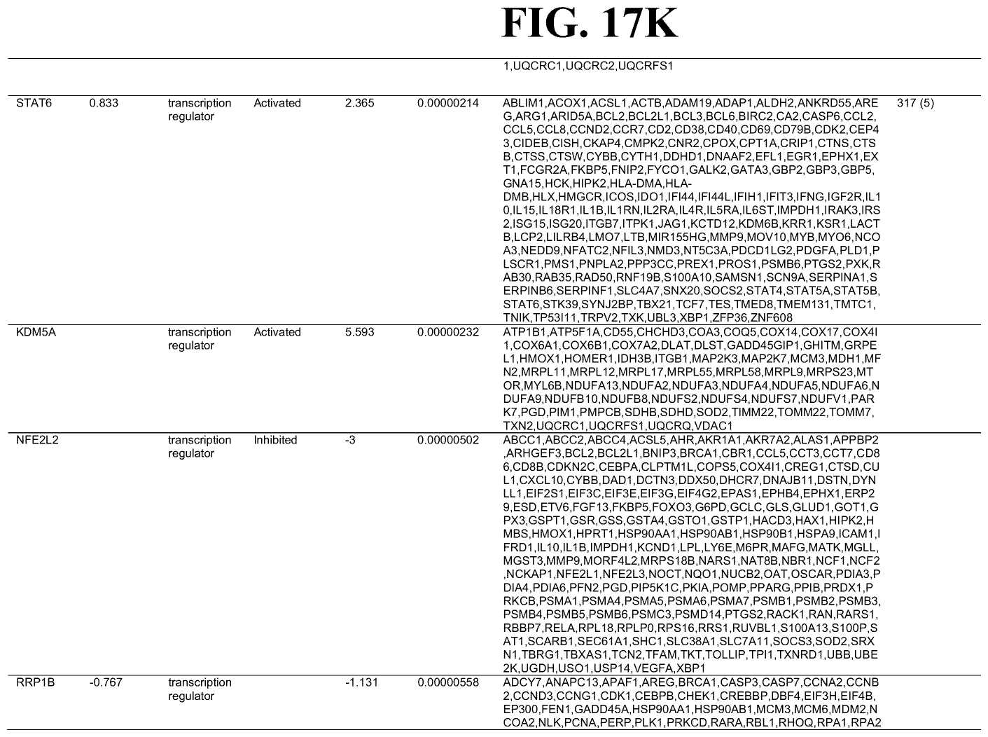

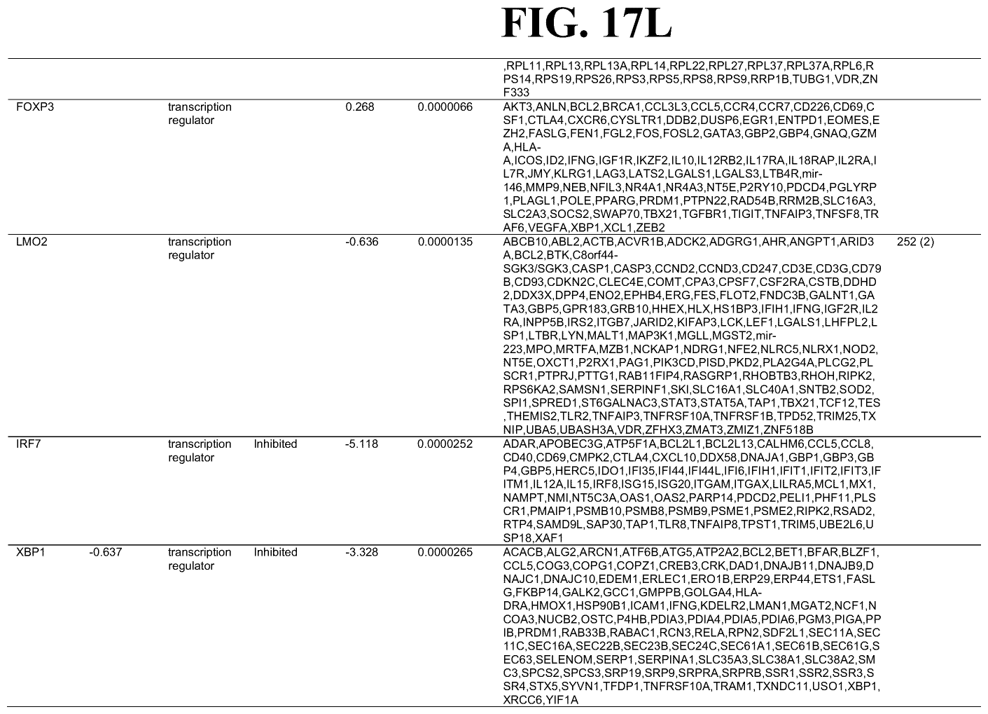

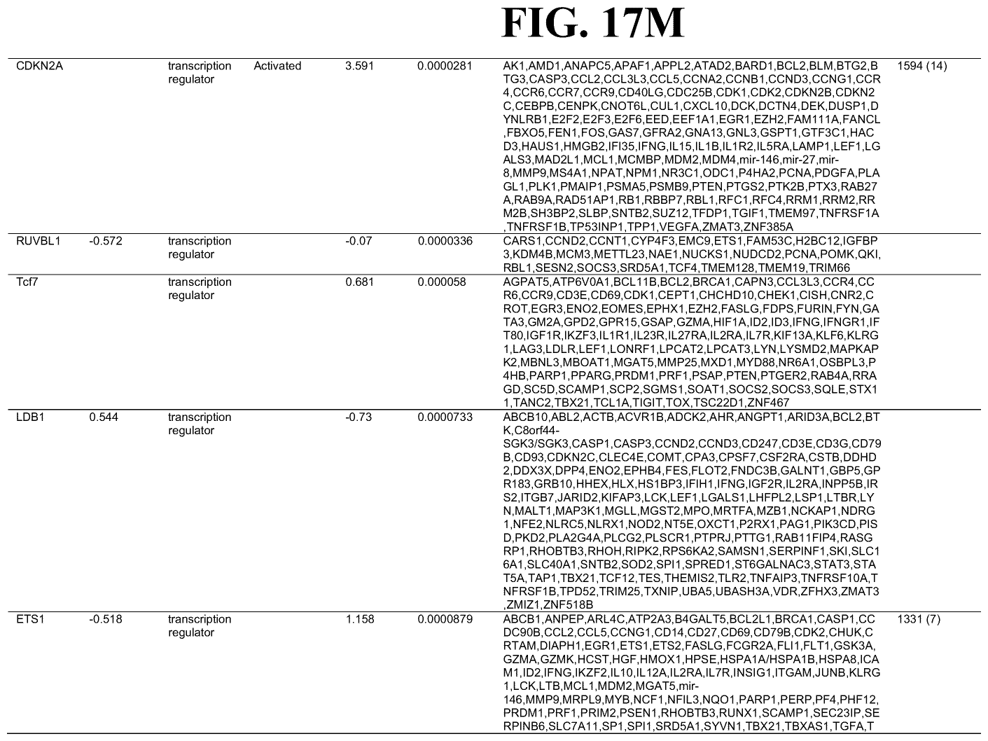

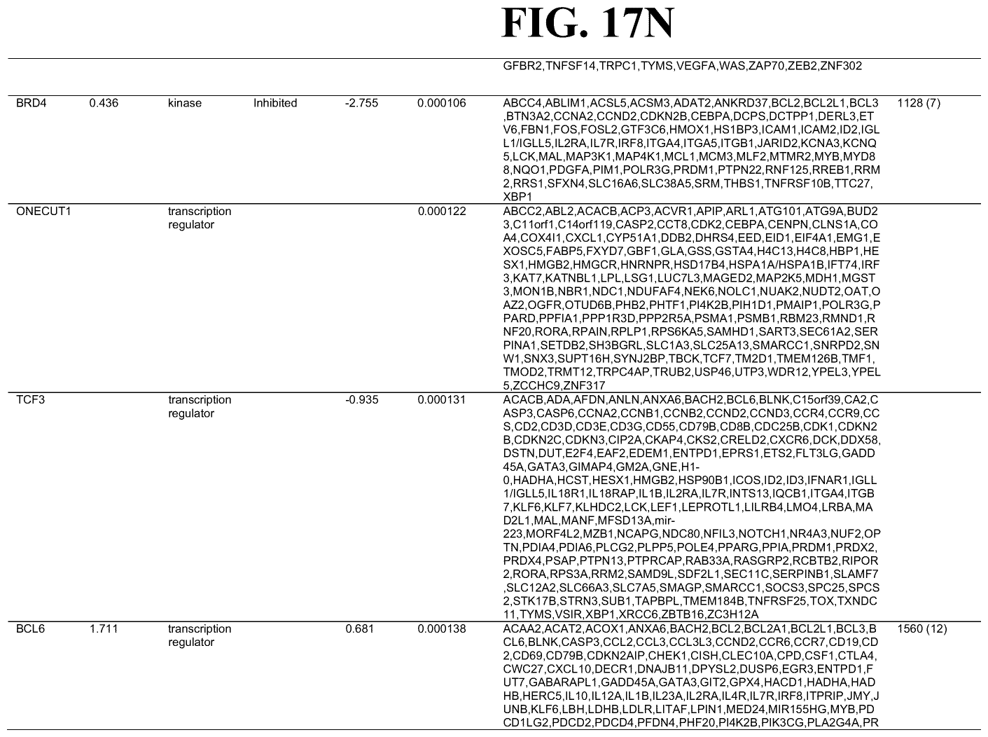

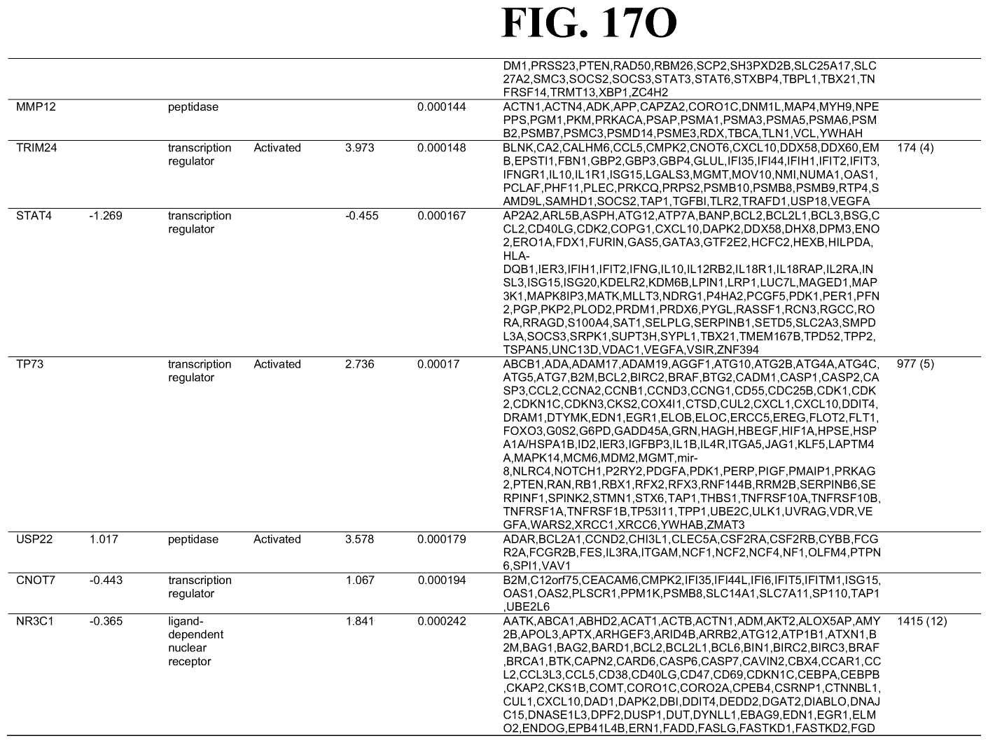

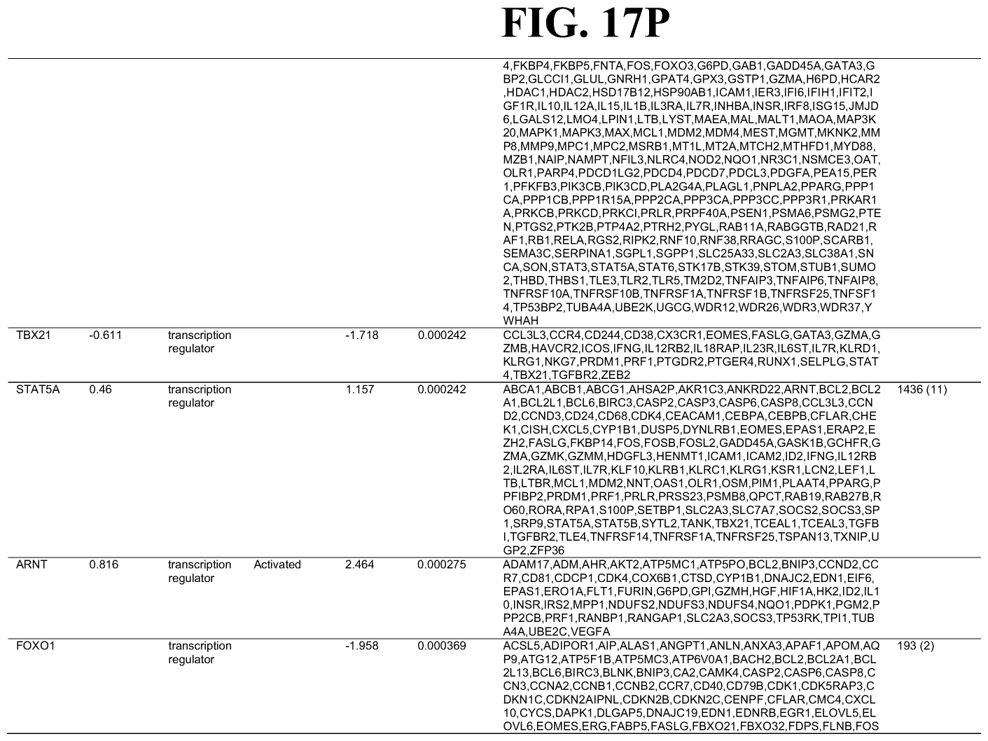

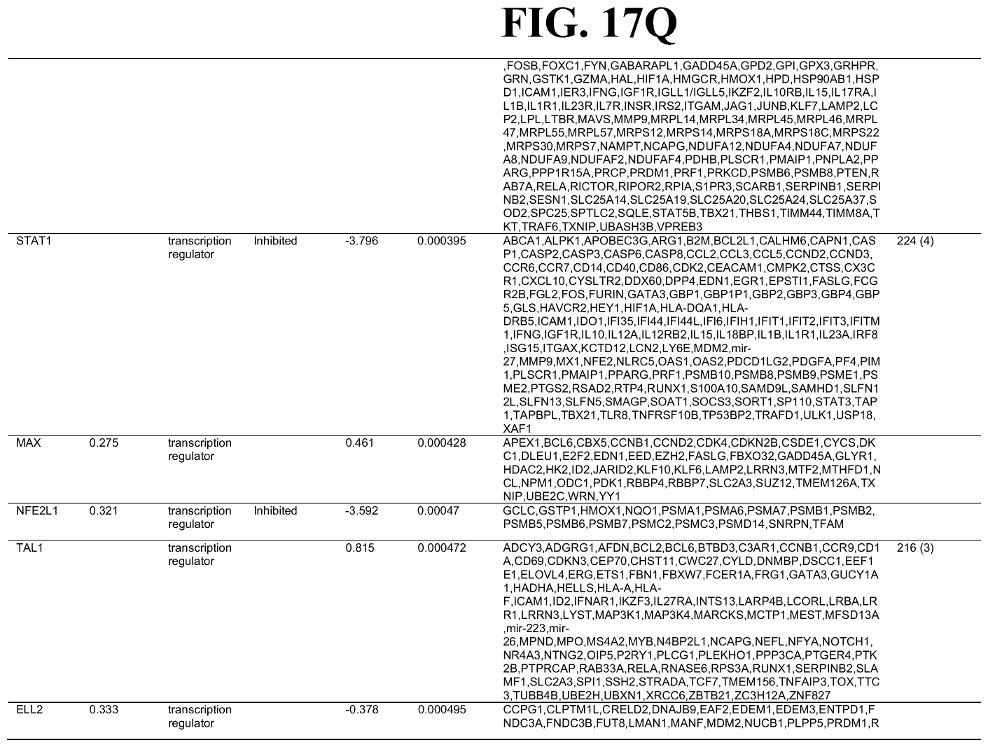

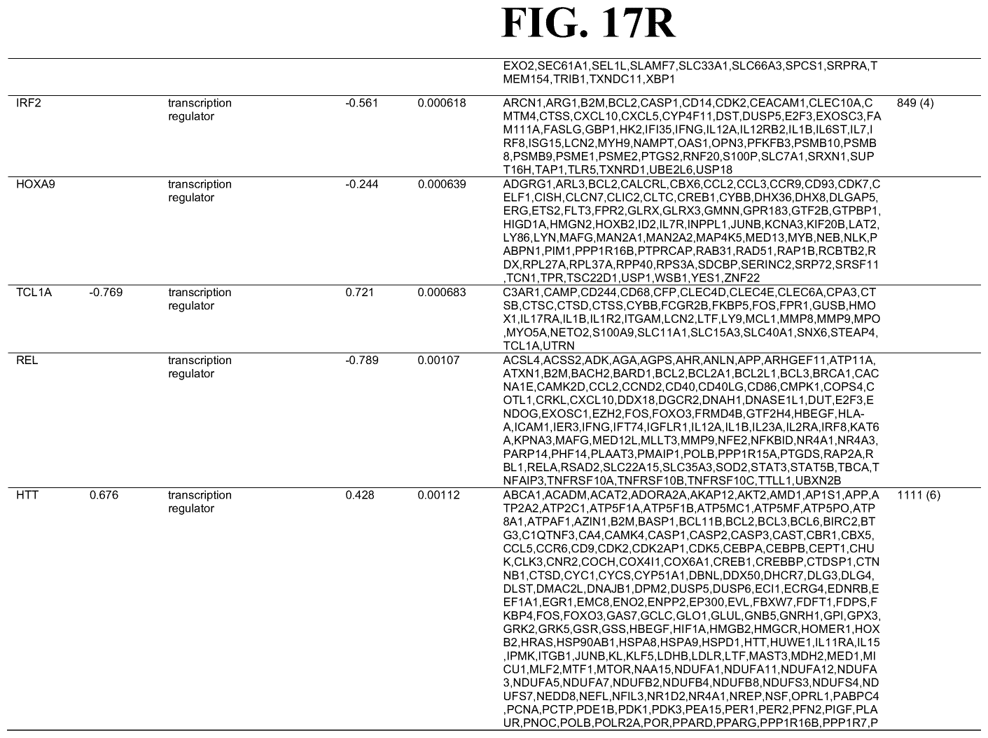

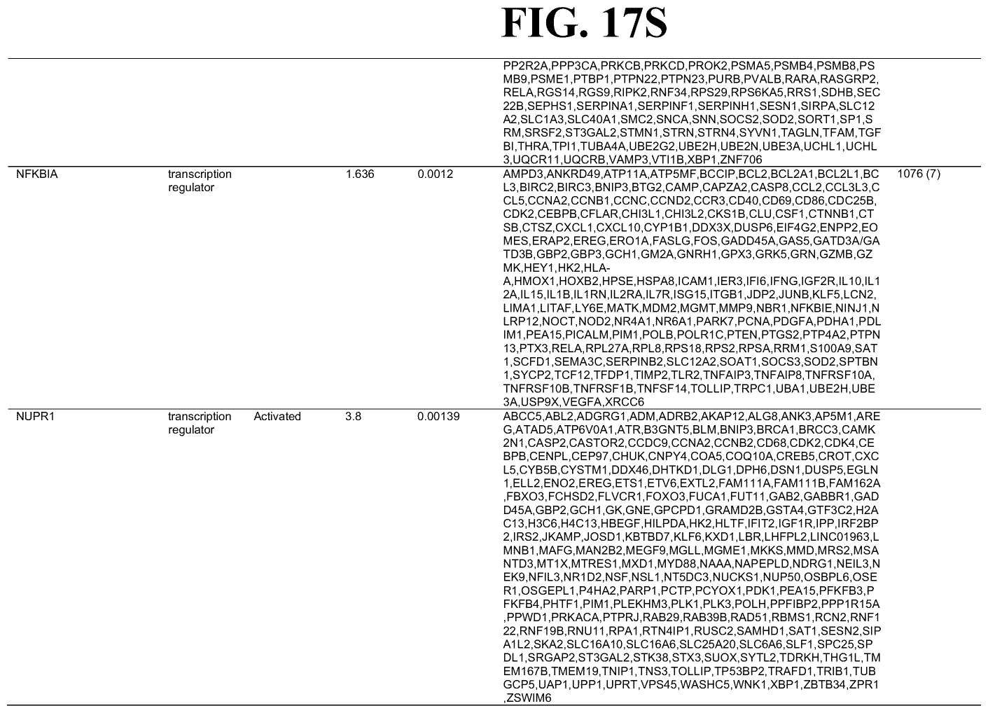

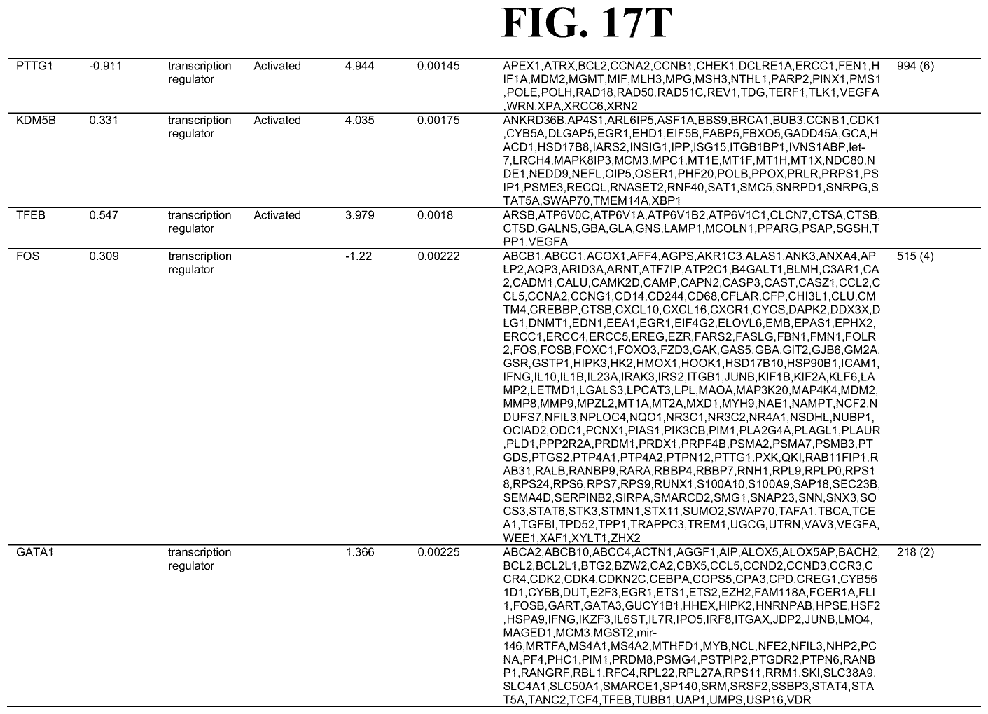

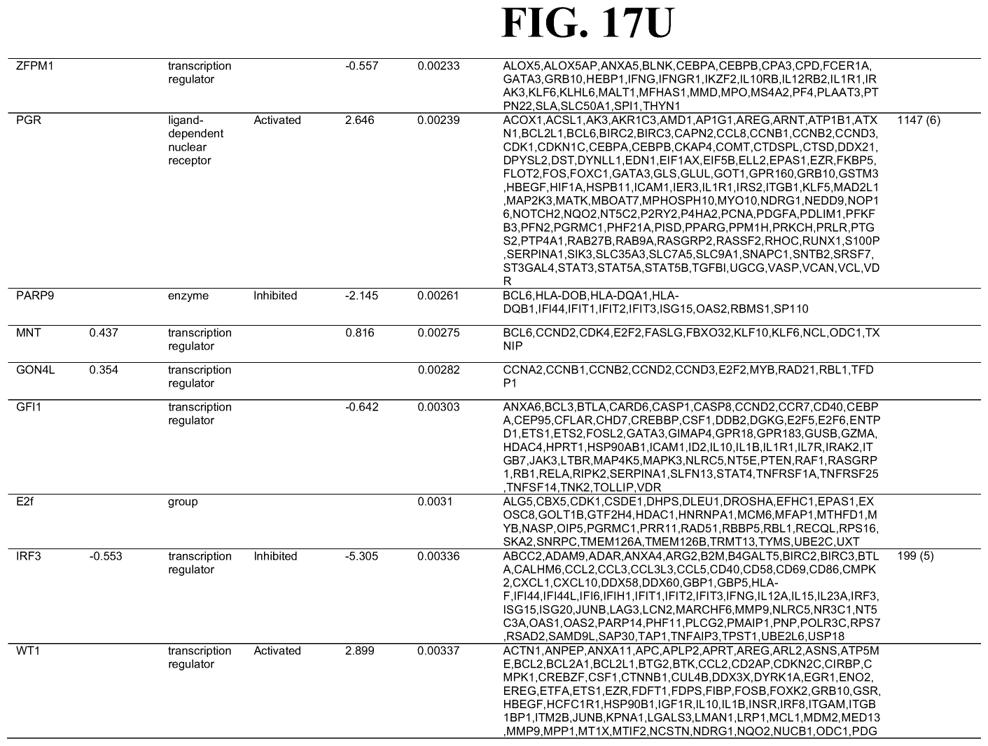

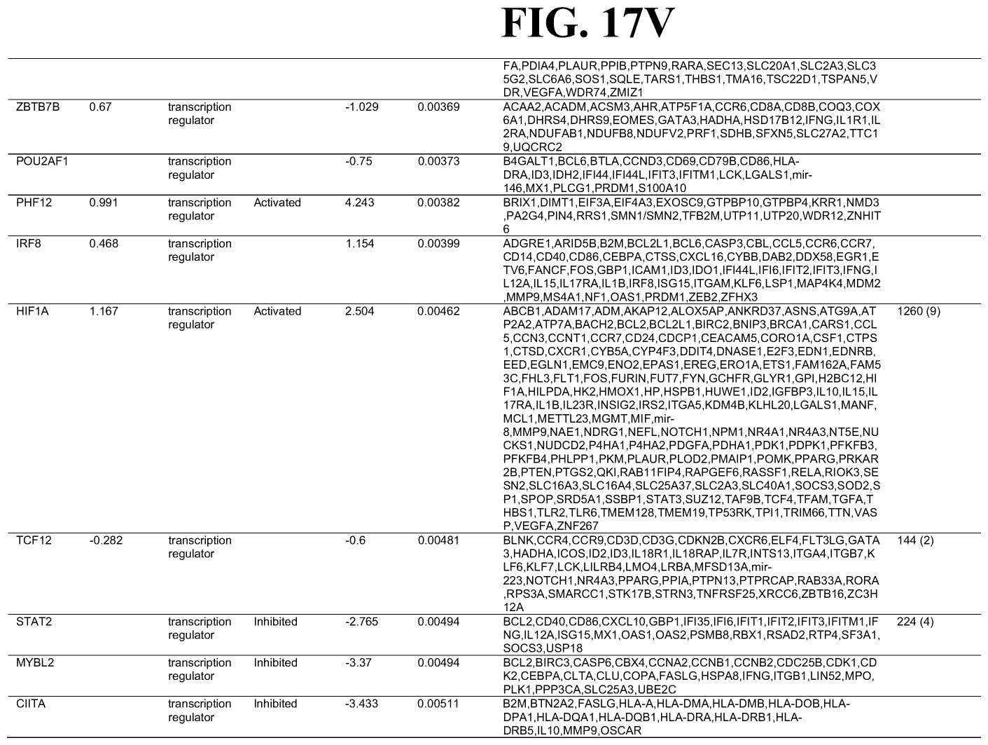

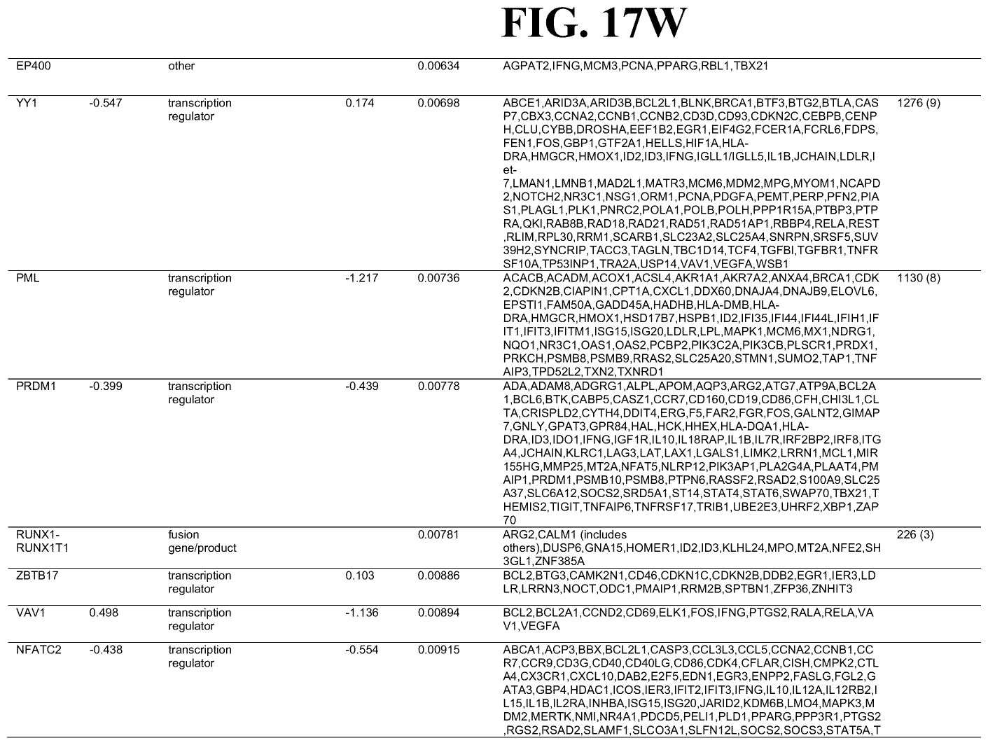

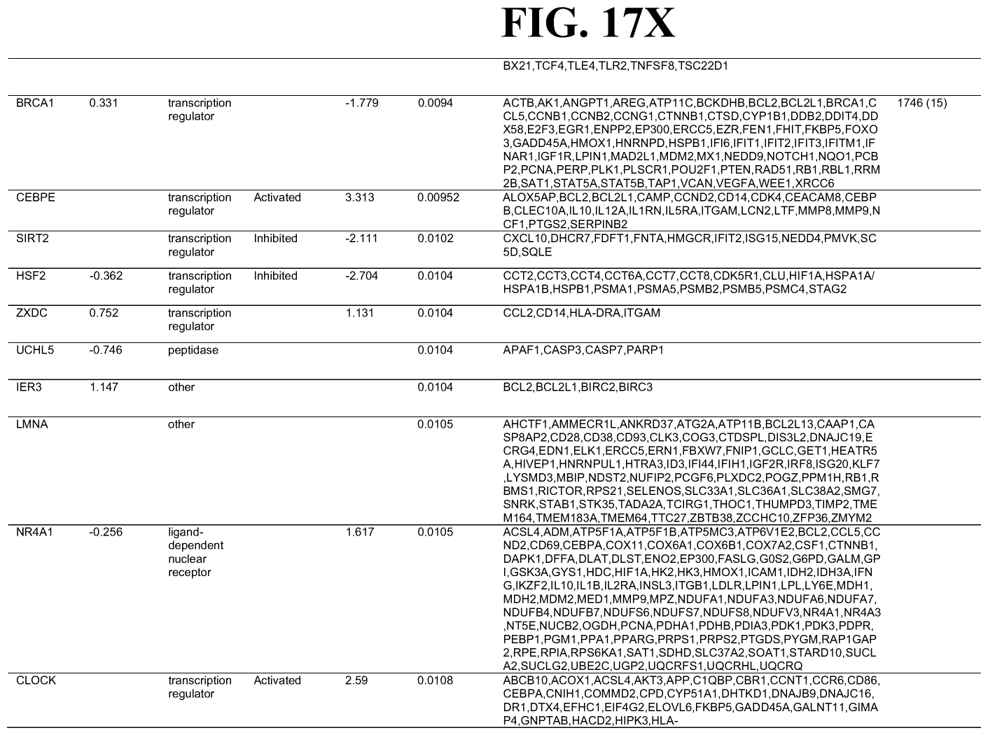

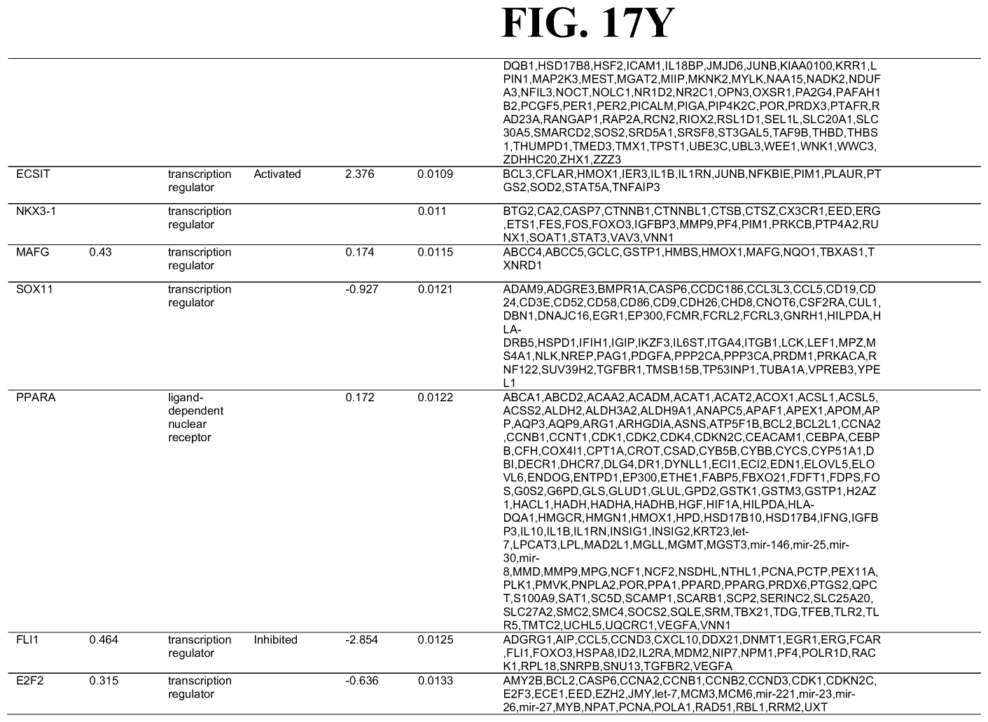

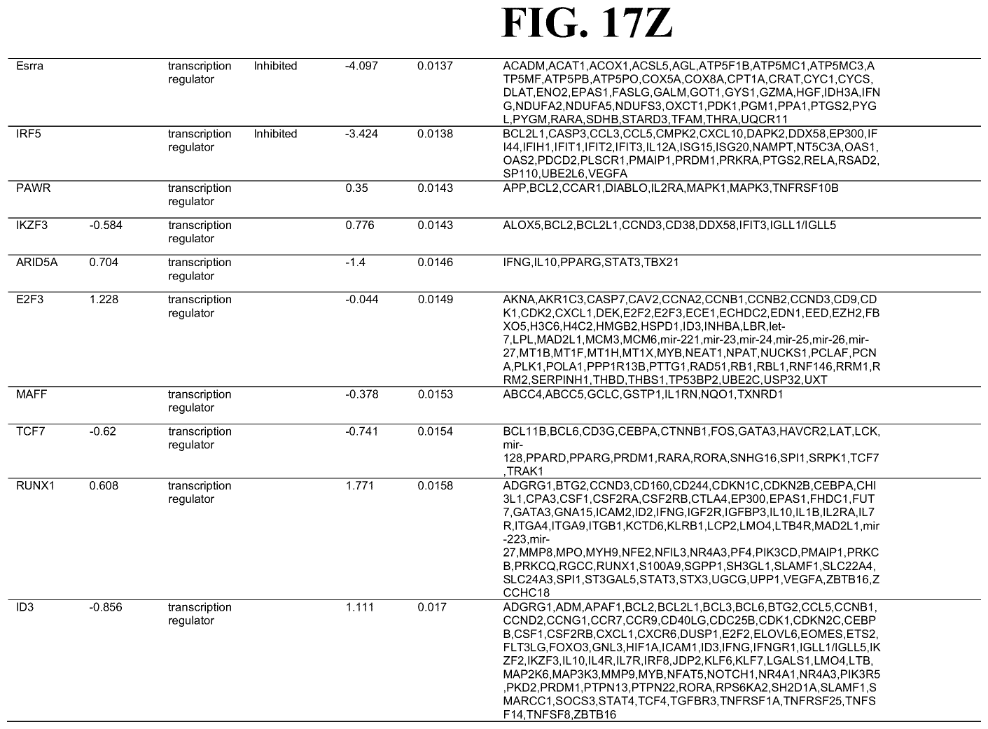

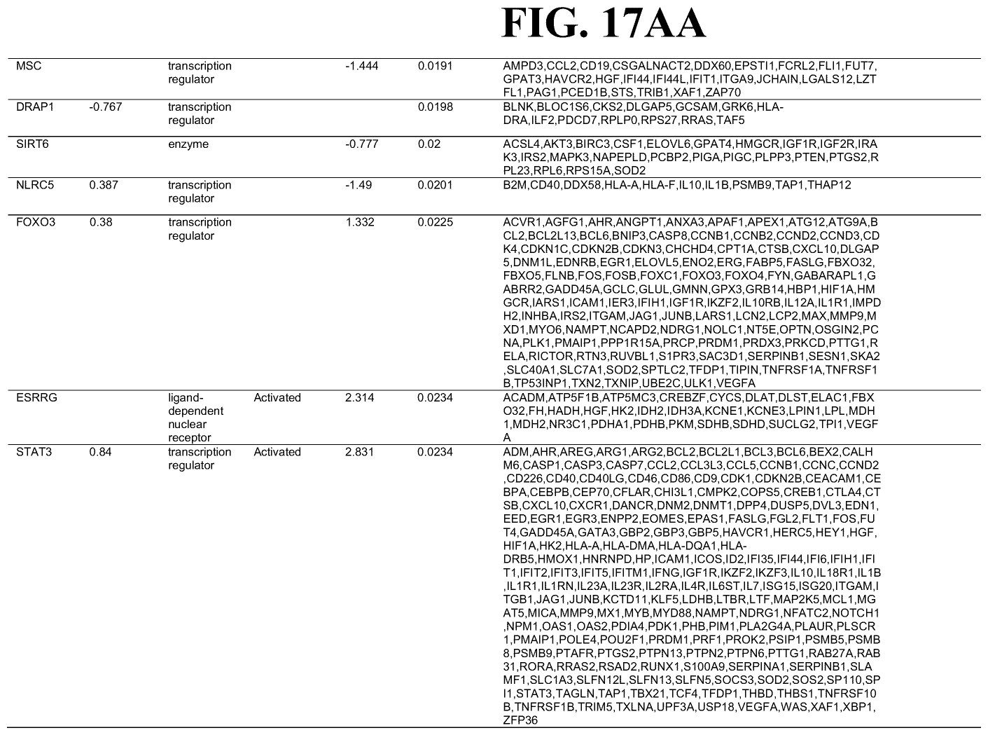

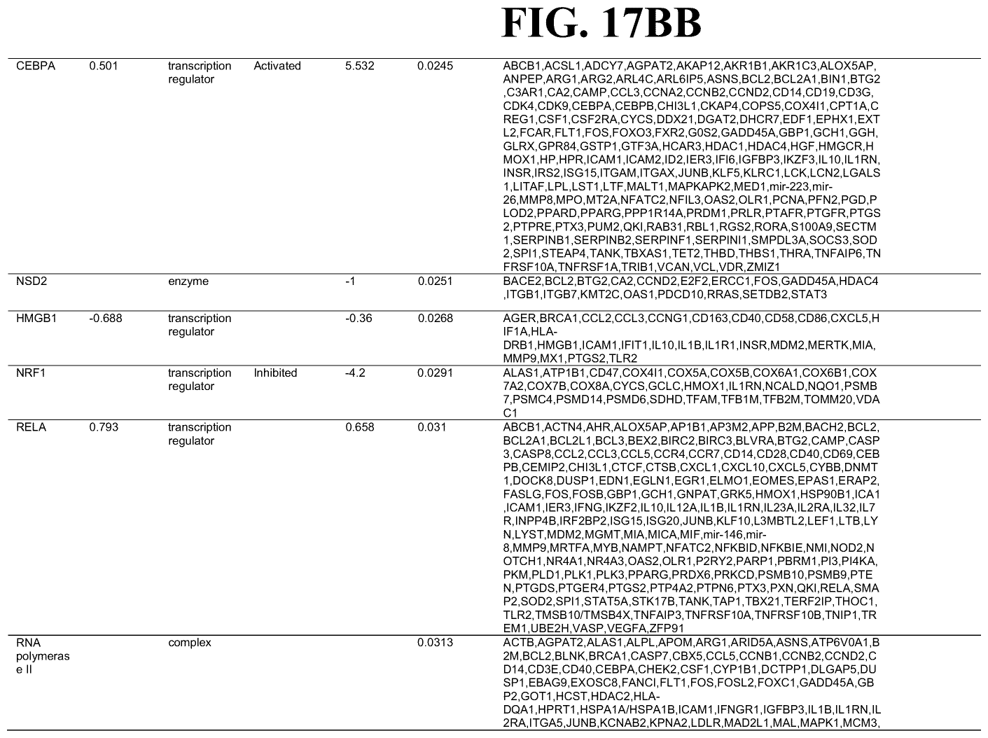

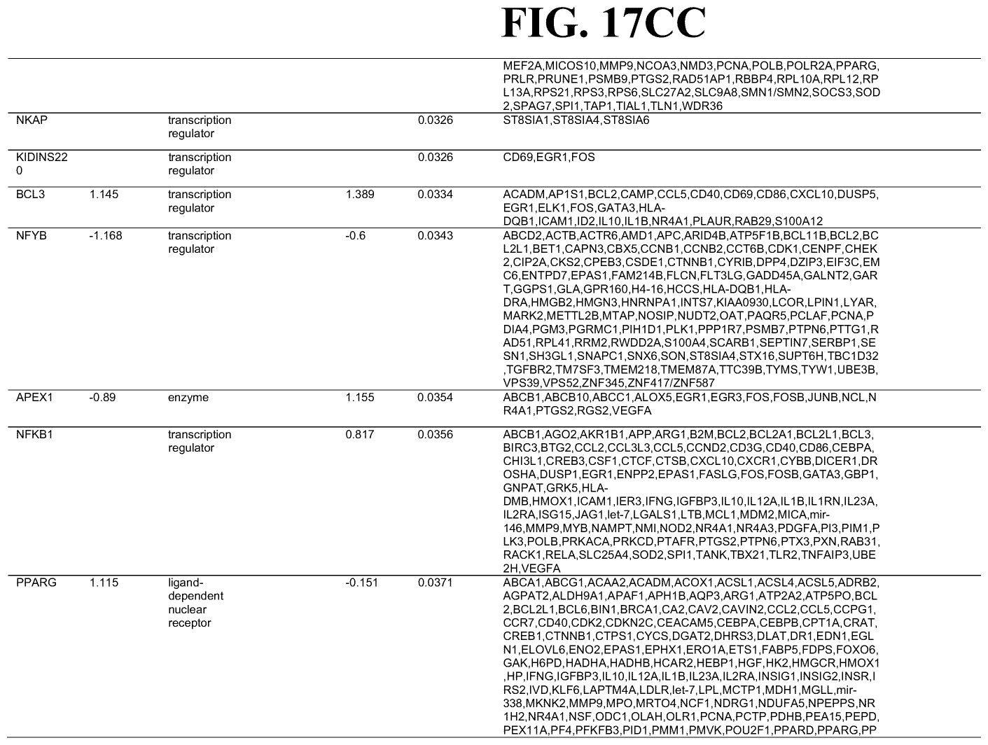

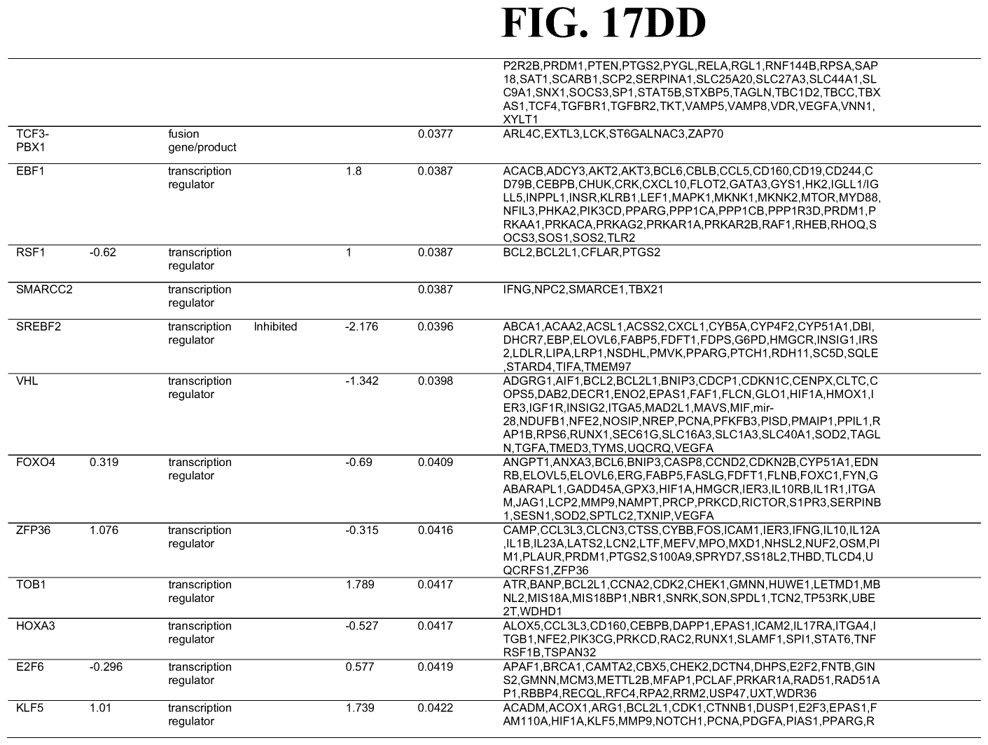

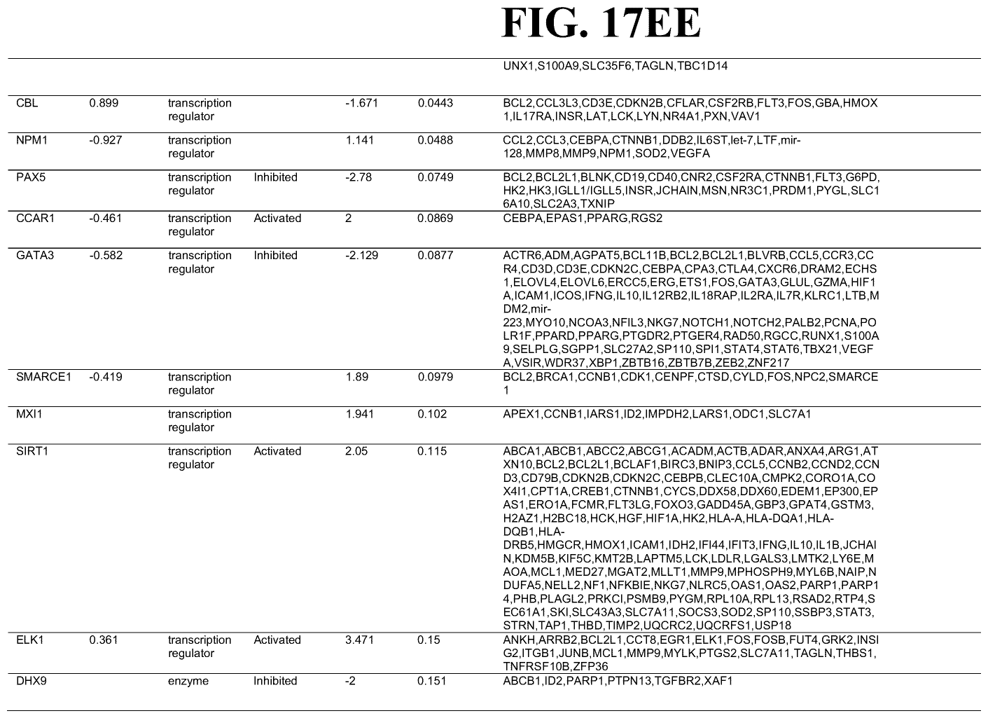

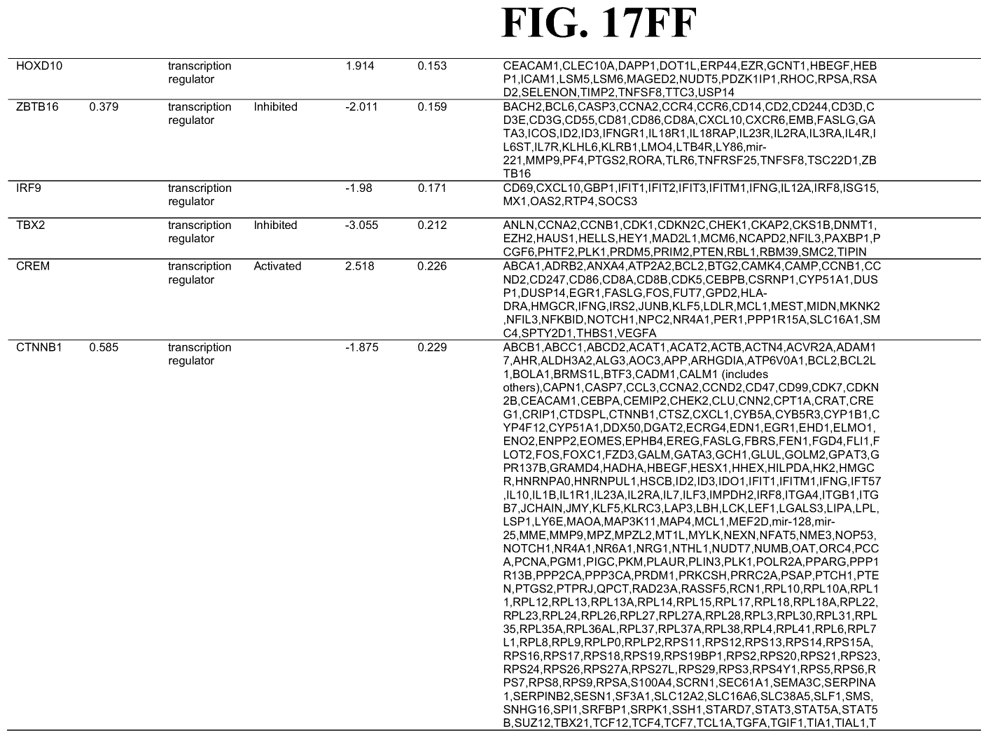

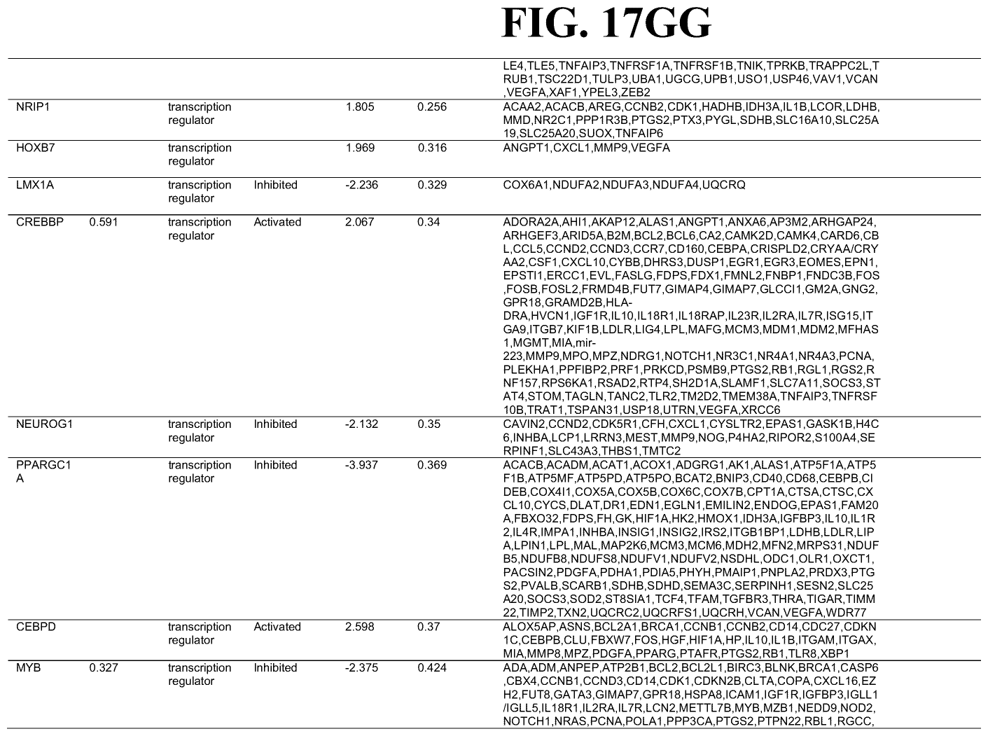

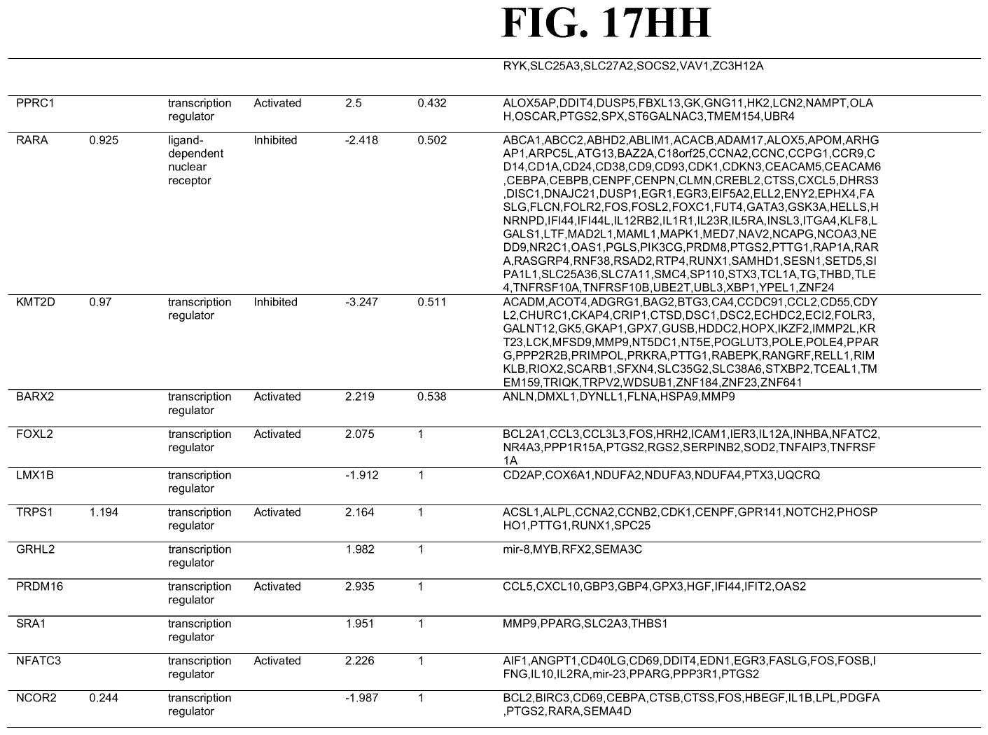

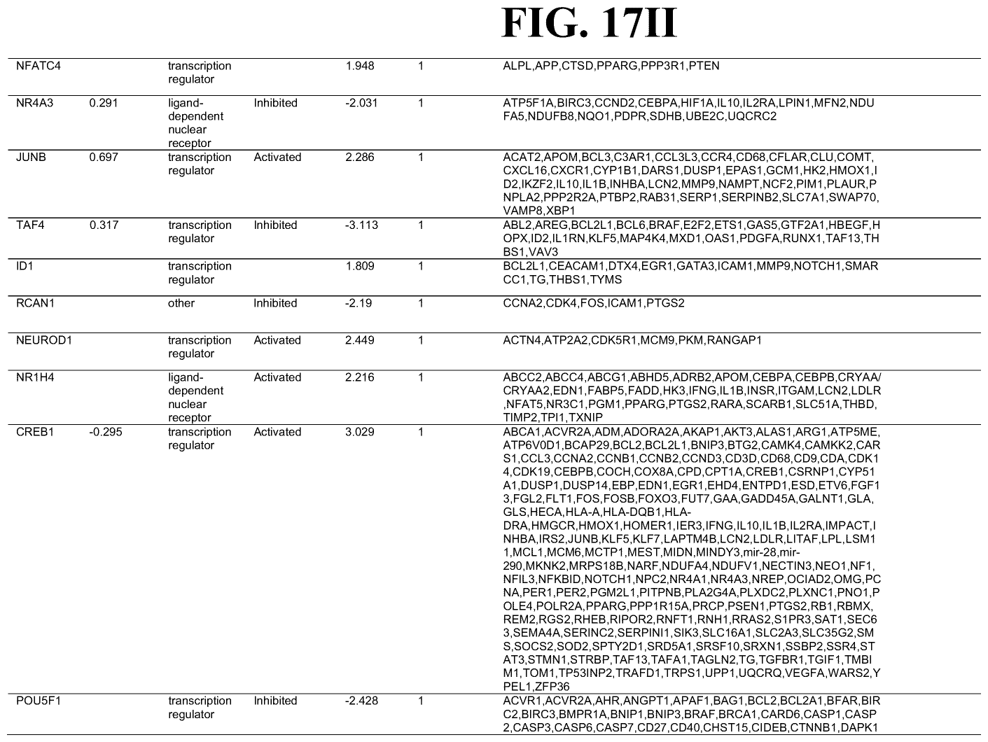

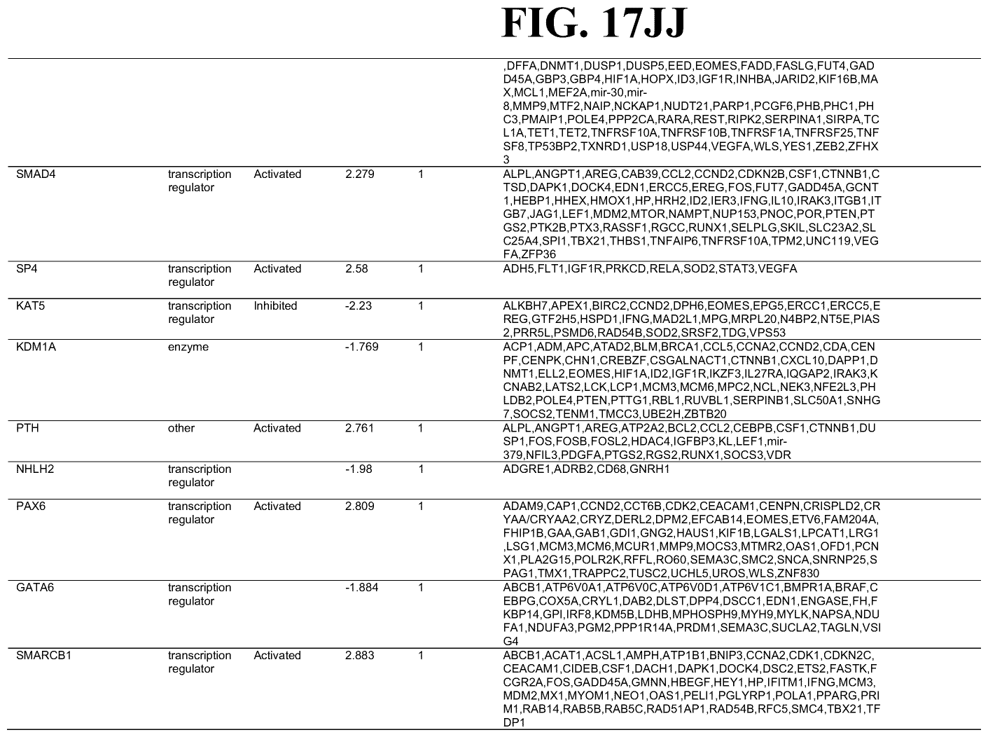

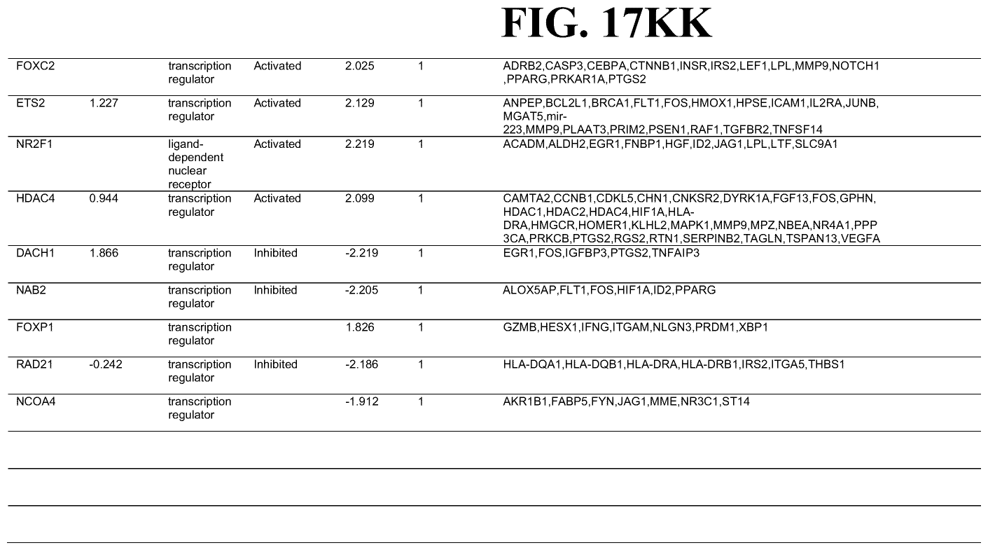

Gene expression analysis demonstrated marked enhancement of genes encoding for HSPs, cochaperones, and chaperonins, particularly the heat inducible HSPs, indicating a robust HSR. Comparing the observed gene expression in a unique cohort of subjects exposed to the same extreme environmental conditions resulting in distinct phenotypes, i.e., heatstroke and heat stress, allowed identification of the pathogenic molecular mechanisms of heatstroke. Supplementary Tables 1 and 4 appear below. Supplementary Tables 2A, 2B, 3A and 3B appear in FIGS. 14A-14E, 15A-15F and 16A-16CC and 17A-17KK, respectively. These and the other figures and tables herein form an integral part of this disclosure.

Supplementary Table 1.
The sequences of the primers used for the validation of the differential
expressed genes identified by the microarray.

| SEQ | Gene | | SEQ ID | |
|---|---|---|---|---|
| ID NO | ID | Forward 5′-3′α | NO | Reverse 5′-3′ |
| 1 | GAPDH | GAAGGTGAAGGTCGGAGTC | 25 | GAAGATGGTGATGGGATTTC |
| 2 | ARG1 | CCCTTTGCTGACATCCCTAA | 26 | GACTCCAAGATCAGGGTGGA |
| 3 | ARG2 | GACACTGCCCAGACCTTTGT | 27 | CGTTCCATGACCTTCTGGAT |
| 4 | MMP8 | CCAGTTTGACATTTGATGCTATCAC | 28 | CTGAGGATGCCTTCTCCAGAA |
| 5 | MMP9 | GCCCCCCTTGCATAAGGA | 29 | CAGGGCGAGGACCATAGAG |
| 6 | HSPA1A | GCCTTTCCAAGATTGCTGTT | 30 | TCAACATTGCAAACACAGGA |
| 7 | PNLDC1 | TATCCCAGTATCCGACCTCCC | 31 | TGTTCCGCGCATCCTTAAAC |
| 8 | FKBP4 | TGACTCCAGTCTGGATCGCAAG | 32 | CTGGTTTGCAGGTGATGTGGCA |
| 9 | HSPH1 | AGGAGTTCCATATCCAGA A | 33 | CAGCTCAACATTCACCAC |
| 10 | BMX | CAGATTGTCTATAAAGATGGGC | 34 | TGTAATGCTTTCAACCACTG |
| 11 | FFAR2 | GTAGCTAACACAAGTCCAGTCCT | 35 | CTAGGTGTTGCTTTGAAGCTTGT |
| 12 | LGALS2 | GGGCAAGAACAACGGGAAGATC | 36 | CCTGTTGGGAAAAGTCAGCTCG |
| 13 | CXCL8 | CTTGGCAGCCTTCCTGATTT | 37 | TTCTTTAGCACTCCTTGGCAAAA |
| 14 | COMMD3 | GTTTCTTGGCGCTTGGAATA | 38 | CCCACCAAGTCCTGTAATTGTT |
| 15 | VNN1 | GGCATTTGACGGACTGCACACT | 39 | CGAAAGTGCCACTGAGGGAGAA |
| 16 | TRDC | CTGGGGGATACGCCGATAAAC | 40 | CCACTGGGAGAGATGACAATAGC |
| 17 | MGAM | CTCCTCATCACTCCAGTTCTGG | 41 | TGCTTCCTCCATCTCACTTGGC |
| 18 | CD3D | GTCATTGCCACTCTGCTCCTTG | 42 | CCTGGTCATTCCTCAACAGAGC |
| 19 | GZMK | TCCAGTATGGCGGACATCACGT | 43 | CGCCTAAAACCACAGTGGGAGA |
| 20 | TIGAR | ACTCAAGACTTCGGGAAAGGA | 44 | CACGCATTTTCACCTGGTCC |
| 21 | SCO2 | GACCACTCCATTGCCATCTACC | 45 | CTCAAGACAGGACACTGCGGAA |
| 22 | P53 | CAGCACATGACGGAGGTTGT | 46 | TCATCCAAATACTCCACACGC |
| 23 | SLC5A3 | AGCACCGTGAGTGGATACTTC | 47 | CCCTGACCGGATGTAAATTGG |
| 24 | RPL22 | AAAGTGAACGGAAAAGCTGGG | 48 | TCACGGTGATCTTGCTCTTGC |

SUPPLEMENTARY TABLE 4
Validation of Microarray gene expression profiling by RT-qPCR

| | Fold-changes (T0) | | Fold-changes (T1) | |
|---|---|---|---|---|
| Gene | Microarray | RT-PCR | Microarray | RT-PCR |
| ARG1 | 2.73 | 1.71 | 4.87 | 3.87 |
| ARG2 | 1.93 | N/A | −0.70 | −0.32 |
| MMP-8 | 4.78 | 3.91 | 4.53 | 4.30 |
| MMP-9 | 2.92 | 1.73 | 4.07 | 3.54 |
| HSPA1A | 6.79 | 3.15 | 2.23 | 1.50 |
| PNLDC1 | 7.40 | 3.18 | 4.19 | 1.90 |
| FKBP4 | 6.02 | 4.06 | 1.96 | 2.01 |
| HSPH1 | 9.32 | 3.61 | 4.66 | N/A |
| BMX | 1.71 | 0.76 | 0.86 | 3.18 |
| FFAR2 | −3.22 | −2.86 | −1.62 | N/A |
| LGALS2 | −2.59 | −2.46 | −1.30 | −2.29 |
| CXCL8 | −2.70 | −1.97 | −1.35 | N/A |
| COMMD3 | −0.67 | −1.66 | −0.33 | −1.35 |
| RPL22 | 0.20 | −1.51 | 0.10 | −1.88 |
| VNN1 | 1.13 | 1.36 | 2.88 | 3.21 |
| TRDC | −0.59 | N/A | −3.85 | N/A |
| MGAM | 0.37 | N/A | 2.42 | 3.24 |
| CD3D | −1.43 | −1.49 | −2.94 | −2.25 |
| GZMK | −0.64 | N/A | −3.20 | −2.26 |
| SLC5A3 | N/A | 3.13 | N/A | N/A |

1. A method for diagnosing a heatstroke in a heatstroke subject, comprising:
detecting an altered expression of two or more RNA biomarkers in a biological sample from the heatstroke subject compared to a control subject not having the heatstroke;
wherein said two or more biomarkers are RNAs encoding one or more proteins selected from the group consisting of HSPA1A, HSPA1B, HSPA6, HSPA4L, HSPB1, DNAJA4, DNAJB1, DNAJB4, and FKBP4 (FKBP Prolyl Isomerase 4).

2. The method of claim 1, wherein the detecting includes quantifying the two or more RNA biomarkers by a multiplex real-time reverse transcription polymerase chain reaction (rRT-PCR) assay.

3. The method of claim 1, wherein the heatstroke is an exertional heatstroke.

4. The method of claim 1, wherein the heatstroke is a non-exertional heatstroke.

5. The method of claim 1, wherein the biological sample comprises a blood product comprising whole blood, buffy coat, peripheral blood mononuclear cells (PBMCs) or other cellular components of blood, plasma, or serum.

6. The method of claim 5, further comprising:
isolating the at least two biomarkers from the blood product of the heatstroke, wherein the heatstroke subject has a rectal, oral, axillary, tympanic or temporal artery body temperature of >37° C.

7. The method of claim 5, further comprising:
isolating the at least two biomarkers from the blood product of the heatstroke subject, wherein the heatstroke subject is experiencing one or more of confusion, agitation, slurred speech, irritability, delirium, seizures and coma, and
wherein the heatstroke subject has a rectal, oral, axillary, tympanic or temporal artery body temperature of >37° C.

8. The method of claim 5, further comprising:
isolating the at least two biomarkers from the blood product of the heatstroke subject, wherein the heatstroke subject has cool, moist skin when in the heat, heavy sweating, faintness, dizziness, fatigue, weak and rapid pulse, low blood pressure upon standing, muscle cramps, nausea, and/or headache, and
wherein the heatstroke subject has a rectal, oral, axillary, tympanic or temporal artery body temperature of >37° C.

9. The method of claim 5, further comprising
isolating the at least two biomarkers from the blood products of the heatstroke subject, wherein the heatstroke subject is exposed to an environment having a heat index of 32° C. to 40° C.

10. The method of claim 5, further comprising:
isolating the at least two biomarkers from the blood product of the heatstroke subject, wherein the heatstroke subject is an amateur athlete, a professional athlete, or a laborer, exposed to an environment having a heat index of >40 to 54° C.

11. The method of claim 5, further comprising:
isolating the at least two biomarkers are isolated from the blood product of the heatstroke subject, wherein the heatstroke subject is an amateur athlete, a professional athlete, or a laborer, exposed to an environment having a heat index of >54° C.

12. The method of claim 1, wherein at least one of the biomarkers encodes a heat shock protein comprising HSPA1A, HSPA1B, HSPA6, or HSPA4L.

13. The method of claim 1, wherein at least one of the biomarkers encodes a heat shock protein comprising HSPB 1.

14. The method of claim 1, wherein at least one of the biomarkers encodes a heat shock protein comprising DNAJA4, DNAJB1, or DNAJB4.

15. The method of claim 1, wherein at least one of the biomarkers encodes a heat shock protein comprising FKBP4 (FKBP Prolyl Isomerase 4).

16. The method of claim 1, wherein the at least two biomarkers encode a heat shock protein comprising at least one of HSPA1A, HSPA1B, HSPA6 or HSPA4L; HSPB1; at least one of DNAJA4, DNAJB1, or DNAJB4; and FKBP4 (FKBP Prolyl Isomerase 4).

17. The method of claim 1, wherein the at least two biomarkers encode heat shock proteins comprising HSPA1A, HSPA1B, HSPA6, HSPA4L; HSPB1; DNAJA4, DNAJB1, DNAJB4; and FKBP4 (FKBP Prolyl Isomerase 4).

18. The method of claim 1, further comprising:
treating the subject with heat stroke by lowering the subject's body temperature.

19. The method of claim 1, further comprising:
treating the subject with heat stroke by administering evaporative, conductive, or convection based cooling to the subject.

20. A composition, comprising:
one or more probes and/or primers that bind to or amplify at least two polynucleotides encoding HSPA1A, HSPA1B, HSPA6, HSPA4; HSPB1; DNAJA4, DNAJB1, DNAJB4; and/or FKBP4 (FKBP Prolyl Isomerase 4).


SEQUENCE LISTING (48 sequences)

SEQ ID NO: 1 - DNA, 19 bp, Homo sapiens
  source 1..19: mol_type = other DNA
gaaggtgaaggtcggagtc

SEQ ID NO: 2 - DNA, 20 bp, Homo sapiens
  source 1..20: mol_type = other DNA
ccctttgctgacatccctaa

SEQ ID NO: 3 - DNA, 20 bp, Homo sapiens
  source 1..20: mol_type = other DNA
gacactgcccagacctttgt

SEQ ID NO: 4 - DNA, 25 bp, Homo sapiens
  source 1..25: mol_type = other DNA
ccagtttgacatttgatgctatcac

SEQ ID NO: 5 - DNA, 18 bp, Homo sapiens
  source 1..18: mol_type = other DNA
gccccccttgcataagga

SEQ ID NO: 6 - DNA, 20 bp, Homo sapiens
  source 1..20: mol_type = other DNA
gcctttccaagattgctgtt

SEQ ID NO: 7 - DNA, 21 bp, Homo sapiens
  source 1..21: mol_type = other DNA
tatcccagtatccgacctccc

SEQ ID NO: 8 - DNA, 22 bp, Homo sapiens
  source 1..22: mol_type = other DNA
tgactccagtctggatcgcaag

SEQ ID NO: 9 - DNA, 19 bp, Homo sapiens
  source 1..19: mol_type = other DNA
aggagttccatatccagaa

SEQ ID NO: 10 - DNA, 22 bp, Homo sapiens
  source 1..22: mol_type = other DNA
cagattgtctataaagatgggc

SEQ ID NO: 11 - DNA, 23 bp, Homo sapiens
  source 1..23: mol_type = other DNA
gtagctaacacaagtccagtcct

SEQ ID NO: 12 - DNA, 22 bp, Homo sapiens
  source 1..22: mol_type = other DNA
gggcaagaacaacgggaagatc

SEQ ID NO: 13 - DNA, 20 bp, Homo sapiens
  source 1..20: mol_type = other DNA
cttggcagccttcctgattt

SEQ ID NO: 14 - DNA, 20 bp, Homo sapiens
  source 1..20: mol_type = other DNA
gtttcttggcgcttggaata

SEQ ID NO: 15 - DNA, 22 bp, Homo sapiens
  source 1..22: mol_type = other DNA
ggcatttgacggactgcacact

SEQ ID NO: 16 - DNA, 21 bp, Homo sapiens
  source 1..21: mol_type = other DNA
ctgggggatacgccgataaac

SEQ ID NO: 17 - DNA, 22 bp, Homo sapiens
  source 1..22: mol_type = other DNA
ctcctcatcactccagttctgg

SEQ ID NO: 18 - DNA, 22 bp, Homo sapiens
  source 1..22: mol_type = other DNA
gtcattgccactctgctccttg

SEQ ID NO: 19 - DNA, 22 bp, Homo sapiens
  source 1..22: mol_type = other DNA
tccagtatggcggacatcacgt

SEQ ID NO: 20 - DNA, 21 bp, Homo sapiens
  source 1..21: mol_type = other DNA
actcaagacttcgggaaagga

SEQ ID NO: 21 - DNA, 22 bp, Homo sapiens
  source 1..22: mol_type = other DNA
gaccactccattgccatctacc

SEQ ID NO: 22 - DNA, 20 bp, Homo sapiens
  source 1..20: mol_type = other DNA
cagcacatgacggaggttgt

SEQ ID NO: 23 - DNA, 21 bp, Homo sapiens
  source 1..21: mol_type = other DNA
agcaccgtgagtggatacttc

SEQ ID NO: 24 - DNA, 21 bp, Homo sapiens
  source 1..21: mol_type = other DNA
aaagtgaacggaaaagctggg

SEQ ID NO: 25 - DNA, 20 bp, Homo sapiens
  source 1..20: mol_type = other DNA
gaagatggtgatgggatttc

SEQ ID NO: 26 - DNA, 20 bp, Homo sapiens
  source 1..20: mol_type = other DNA
gactccaagatcagggtgga

SEQ ID NO: 27 - DNA, 20 bp, Homo sapiens
  source 1..20: mol_type = other DNA
cgttccatgaccttctggat

SEQ ID NO: 28 - DNA, 21 bp, Homo sapiens
  source 1..21: mol_type = other DNA
ctgaggatgccttctccagaa

SEQ ID NO: 29 - DNA, 19 bp, Homo sapiens
  source 1..19: mol_type = other DNA
cagggcgaggaccatagag

SEQ ID NO: 30 - DNA, 20 bp, Homo sapiens
  source 1..20: mol_type = other DNA
tcaacattgcaaacacagga

SEQ ID NO: 31 - DNA, 20 bp, Homo sapiens
  source 1..20: mol_type = other DNA
tgttccgcgcatccttaaac

SEQ ID NO: 32 - DNA, 22 bp, Homo sapiens
  source 1..22: mol_type = other DNA
ctggtttgcaggtgatgtggca

SEQ ID NO: 33 - DNA, 18 bp, Homo sapiens
  source 1..18: mol_type = other DNA
cagctcaacattcaccac

SEQ ID NO: 34 - DNA, 20 bp, Homo sapiens
  source 1..20: mol_type = other DNA
tgtaatgctttcaaccactg

SEQ ID NO: 35 - DNA, 23 bp, Homo sapiens
  source 1..23: mol_type = other DNA
ctaggtgttgctttgaagcttgt

SEQ ID NO: 36 - DNA, 22 bp, Homo sapiens
  source 1..22: mol_type = other DNA
cctgttgggaaaagtcagctcg

SEQ ID NO: 37 - DNA, 23 bp, Homo sapiens
  source 1..23: mol_type = other DNA
ttctttagcactccttggcaaaa

SEQ ID NO: 38 - DNA, 22 bp, Homo sapiens
  source 1..22: mol_type = other DNA
cccaccaagtcctgtaattgtt

SEQ ID NO: 39 - DNA, 22 bp, Homo sapiens
  source 1..22: mol_type = other DNA
cgaaagtgccactgagggagaa

SEQ ID NO: 40 - DNA, 23 bp, Homo sapiens
  source 1..23: mol_type = other DNA
ccactgggagagatgacaatagc

SEQ ID NO: 41 - DNA, 22 bp, Homo sapiens
  source 1..22: mol_type = other DNA
tgcttcctccatctcacttggc

SEQ ID NO: 42 - DNA, 22 bp, Homo sapiens
  source 1..22: mol_type = other DNA
cctggtcattcctcaacagagc

SEQ ID NO: 43 - DNA, 22 bp, Homo sapiens
  source 1..22: mol_type = other DNA
cgcctaaaaccacagtgggaga

SEQ ID NO: 44 - DNA, 20 bp, Homo sapiens
  source 1..20: mol_type = other DNA
cacgcattttcacctggtcc

SEQ ID NO: 45 - DNA, 22 bp, Homo sapiens
  source 1..22: mol_type = other DNA
ctcaagacaggacactgcggaa

SEQ ID NO: 46 - DNA, 21 bp, Homo sapiens
  source 1..21: mol_type = other DNA
tcatccaaatactccacacgc

SEQ ID NO: 47 - DNA, 21 bp, Homo sapiens
  source 1..21: mol_type = other DNA
ccctgaccggatgtaaattgg

SEQ ID NO: 48 - DNA, 21 bp, Homo sapiens
  source 1..21: mol_type = other DNA
tcacggtgatcttgctcttgc